(19) United States
(12) Patent Application Publication
(10) Pub. No.: US 2010/0286233 A1
(43) Pub. Date: Nov. 11, 2010

(54) PERIPHERAL AND NEURAL INFLAMMATORY CROSSTALK

(75) Inventors: Stephanos Kyrkanides, Rochester, NY (US); M. Kerry O'Banion, Pittsford, NY (US); Ross H. Tallents, Rochester, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: University of Rochester, Rochester, NY (US)

(21) Appl. No.: 12/282,251

(22) PCT Filed: Mar. 9, 2007
(86) PCT No.: PCT/IB07/04351

§ 371 Date: Jul. 30, 2010

Related U.S. Application Data
(60) Provisional application No. 60/780,734, filed on Mar. 9, 2006.
(60) Provisional application No. 60/807,481, filed on Jul. 15, 2006.

(51) Int. Cl. A61K 31/713; A61K 31/7088; A61P 19/00; A61P 25/28; A61P 29/00

(52) U.S. Cl. 514/44 A; 514/44 R

(57) ABSTRACT

Disclosed are compositions and methods for the study and treatment of inflammatory disease, neurological disorders, bone disease, pain, and methods of making and using thereof.

### I. CROSS-REFERENCE TO RELATED APPLICATIONS

This application claims benefit of U.S. Provisional Application No. 60/780,734, filed Mar. 9, 2006 and U.S. Provisional Application No. 60/807,481, filed Jul. 15, 2006, which are hereby incorporated herein by reference in their entirety.

### II. BACKGROUND

There are a number of diseases and disorders related to pain and inflammation, as well as a number of pathways and molecules related to pain and inflammation. Disclosed are methods of treating pain, neurological disorders, bone disease, and inflammatory disease using compositions and methods identified herein.

### III. SUMMARY

In accordance with the purposes of this invention, as embodied and broadly described herein, this invention, in one aspect, relates to vector constructs that can be used to inhibit inflammation and treat subjects with inflammatory disease, bone disease, and pain.

Disclosed are methods and compositions related to polypeptides, nucleic acids, vectors, cells, and transgenic animals for the study and treatment of inflammatory disease, neurological disorders, bone disease, pain, and methods of making and using thereof.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows β-hexosaminidase deficiency results in the development of craniofacial skeletal defects. Craniofacial morphology was evaluated in HexB−/− knockout (N=6), HexB+/− heterozygotes (N=6) and wild type (N=7) knockout mice. Lateral cephalometric radiographs of (A) HexB−/− and (B) wild type mice are shown (arrow points to cranial base). In addition, HexB−/− knockout mice (N=10) treated with a single FIV(HEX) intra-peritoneal injection at postnatal day P4 were included in the study. (C) Cephalometric analyses revealed that (D) the Ba—Rh, (E) Na—Rh and (F) Na—Ra distances were significantly reduced in the HexB−/− knockout mice compared to HexB+/−, wild type controls as well as FIV(HEX)-treated mice. These measurements demonstrate the presence of cranial base and nasomaxillary deficiency in mice suffering from β-hexosaminidase deficiency. The data also show that neonatal β-hexosaminidase restitution rescued the HexB−/− knockout mice from developing craniofacial dysplasia. Differences between the 4 groups were evaluated by one way analysis of variance followed by post-hoc analysis using the Dunnets method. Ba—Rh differences were statistically different at p=0.00282 (power=0.8913), for Na—Rh p=0.0020 (power=0.9212) and Na—Ba p=0.0021 (power=0.91). mean+/− SD. *p<0.05

FIG. 2 shows cellular organization and chondrocyte maturation are impaired secondary to β-hexosaminidase deficiency in the spheno-occipital synchondrosis (SOS). Histological sections of the cranial base harvested from wild type, HexB−/− and FIV(HEX)-treated knockout mice were first analyzed by Alcian Blue—Orange G histochemistry. This analysis revealed (A) the presence of “growth plate” cartilage (light blue stain) in the proliferative zone of wild type mice. Conversely, (B) HexB−/− knockout mice displayed reduced levels of cartilage and instead presented ectopic bone formation (red stain). (B) Neonatal FIV(HEX) treatment restored the SOS cyto-architecture. Collagen-2 immunohistochemistry (brown stain—hematoxylin nuclear counter-stain) was employed to evaluate the presence of chondrocytes in the SOS. (D) Collagen-2 expression was primarily localized in the proliferative/hypertrophic zones of wild type SOS. (E) In contrast, Col-2 staining in HexB−/− mice further demonstrated abnormal SOS cyto-architecture and loss of the resting zone. (F) Neonatal FIV(HEX) treatment restored the SOS cyto-architecture as evaluated by Col-2 immunohistochemistry. (G) PTHrP expression, a known inhibitor of chondrocyte differentiation, was detected in the proliferative zone of wild type mice and (H) was completely eliminated in the HexB−/− SOS. In contrast, (I) FIV(HEX)-treated knockout mice showed partial restoration of PTHrP expression in the synchondrosis. (J) Tissue reactive alkaline phosphatase (TRAP; pink stain—hematoxylin nuclear counter-stain) is normally absent from proliferative chondrocytes and is present only in areas of bone formation/remodeling. (K) A remarkable upregulation of TRAP activity was observed in the SOS proliferative zone, (L) which was completely ameliorated in the FIV(HEX)-treated knockout mice. (M) FITC-immunofluorescence (green) for vascular endothelial growth factor (VEGF), a marker of terminally differentiated chondrocytes, demonstrated the lack of terminally mature chondrocytes in the SOS of wild type mice. However, (N) a large number of VEGF positive cells were observed in the proliferative zone of HexB knockout mice, which (O) diminished in FIV(HEX)-treated mice. In contrast, the HexA−/−/B+/− knockout mice lacked VEGF expression in the synchondroses. Quantitative evaluation of these analyses is presented in FIG. 4A. r: resting zone; p: proliferative zone; h: hypertrophic zone; m: bone marrow; b: bone. Bar=100 μm

FIG. 3 shows growth plate phenotype is affected in β-hexosaminidase deficient mice. Histological sections of the femur and tibia harvested from wild type, HexB−/− and FIV(HEX)-treated knockout mice were analyzed by Alcian Blue—Orange G histochemistry. (A) It revealed the presence of a resting zone flanked by woven bone in wild type mice compared to (B) a wider zone of chondrocytes and bone layer in the knockout mice. (C) Neonatal FIV(HEX) treatment of knockout mice moderated the amount of woven bone adjacent to the growth plate chondrocytes. These findings were further confirmed by Col-2 immunohistochemistry evaluating the presence of chondrocytes in long bone growth plates. Interestingly, an increase in the size of chondrocyte columns were observed in the knockout mice compared to wild type and virally transduced mice. (G) Tissue reactive alkaline phosphatase (TRAP; pink stain—hematoxylin nuclear counter-stain) is normally absent from proliferative chondrocytes and is present only in areas of bone formation/remodeling. (K) A remarkable upregulation of TRAP activity was observed in the growth plate, (L) which was partially ameliorated in the FIV(HEX)-treated knockout mice. Moreover, COX-2 expression was evaluated by immunohistochemistry in the femur and SOS of wild type mice (J & M, respectively), HexB−/− knockout mice (K & N, respectively) and FIV(HEX)-treated knockout mice (L & O, respectively). COX-2 expression in chondrocytes was induced in the presence of β-hexosaminidase deficiency and subsequently can be resolved following neonatal β-hexosaminidase, a finding suggesting a direct link between β-hexosaminidase and the cyclooxygenase-prostaglandin pathway. Quantitative evaluation of these analyses is presented in FIG. 4B. Bar=100 μm.

FIG. 4 shows COX-2 activation is implicated in abnormal chondrocyte maturation secondary to β-hexosaminidase deficiency. Quantitative analysis of the phenotypic changes observed in the (A) SOS and (B) growth plates of wild type, HexB−/− and FIV-treated knockout mice. The number of COX-2 positive cells was significantly increased in the knockout mice secondary to the attendant metabolic disorder. (C) The stress-activated p38 MAK, a known inducer of COX-2, was also induced in proliferative zone chondrocytes of HexB−/− mice compared to (D) wild type mice as assessed by immunofluorescence. The expression of COX-2, previously implicated in chondrocyte differentiation and maturation, was elevated (E) in the HexB−/− versus (F) wild type mice detected by immunofluorescence. The expression of the EP2 receptor was confirmed in the SOS of (G) HexB−/− and (H) wild type mice, a distal member of the cyclooxygenase-prostaglandin pathway previously implicated in chondrocyte maturation. To test whether chondrocyte maturation and differentiation are affected by the cyclooxygenase-prostaglandin pathway, the C2C12 cell line, an in vitro model of chondrocyte differentiation was employed. To this end, the conversion of immature C2C12 cells to an osteoblastic phenotype was evaluated by assessing alkaline phosphatase expression in situ (black stain). (I) Naïve cells showed no signs of conversion over a 4 day period. Conversely, (J) treatment with BMP-2 (300 ng/mL) over 4 days induced the expression of alkaline phosphatase (black stain) in 10% of the cells in culture indicating a shift in their differentiation towards osteoblastic cells. Conversely, (K) treatment of C2C12 cells with BMP-2 plus PGE2 (10-8M) over the same time period increased the number of cells expressing alkaline phosphatase by approximately 5-fold, demonstrating the ability of PGE2 to increase the differentiation rate of C2C12 to osteoblastic cells (number of cells converted in a defined period of time). mean+/−SD. *p<0.05. Bar=100 μm.

FIG. 5 shows diagram of neural inflammatory crosstalk.

FIG. 6 shows murine IL-1β (2 ng in 2 μl of normal saline) was injected transdermally in the cisterna magna of deeply anesthetized C57BL/6 mice (anesthetic: ketamine 40 mg/kg IP). Two days later, the mice were sacrificed, transfused transcardially with 4% paraformaldehyde in phosphate buffered saline solution and the brain stem was harvested, frozed and cut at 18 μm thick horizontal sections which were collected on glass slides. The histology slides were then analyzed by immunohistochemistry (IHC) using antibodies raised against calcitonin gene-related peptide (CGRP; μ33) and glial fibrillary acidic protein (GFAP; Dako). Results showed that IL-1β induced the expression of GFAP and CGRP in the descending trigeminal nucleus (medullary dorsal horn) of these mice.

FIG. 7 shows diagram of centrally-induced pain. Peripheral inflammation, such as in the case of arthritis or other inflammatory disorders, results in activation of the primary sensory nerves, the first component in the transmission of painful stimuli from the periphery to the central nervous system (CNS). Subsequently, at the dorsal horns of the brain stem or spinal cord, the primary sensory fibers synapse and thus activate second order nerve fibers as part of the pain circuit, which ultimately results in the experience of pain. These signals also can also activate resident astrocytes and microglia cells.

FIG. 8 shows transgene structure of GFAP-IL1β^{XAT} used to develop transgenic mice. Injection of FIV(Cre) virus in the brain of ROSA26 reporter mice resulted in activation of the reporter gene lacZ in the area of injection.

FIG. 9 shows two transgenic lines generated for GFAP-IL1β^{XAT}, namely 787-2-1 (designated as mouse line A) and 787-2-2 (line B). Primary astrocyte cultures from line B were treated with FIV(Cre), which resulted in increased expression of transgenic IL1β as assessed by ELISA. There was lack of IL1β in the controls (wild type cells treated with Cre or B cells treated with gfp virus).

FIG. 10 shows injection of FIV(Cre) in the brain of B mice resulted in activation of microglia cells as assessed by major histocompatibility-II (MHC-II) IHC, astrocyte activation as assessed by GFAP IHC. Mouse line A also display induction of these genes but to a lesser degree. FIV(gfp) did not induced any brain inflammation.

FIG. 11 shows monocyte chemo-attractant protein-1 (MCP-1) was also induced in the B mouse line injected with FIV(Cre).

FIG. 12 shows that inflammation is due to IL-1β induction following FIV(Cre) injection in the GFAP-IL1β^{XAT} transgenic mice. GFAP-IL1β^{XAT} mice were crossed into the IL-1 receptor type 1 (IL1R1^{-/-}) knockout mice and the experiment repeated. Deletion of the IL1R1 in the GFAP-IL1β^{XAT} abolished the previously observed brain inflammation.

FIG. 13 shows injection of FIV(Cre) in the cisterna magna of GFAP-IL1β^{XAT} mice (3 μl of a 10⁶ ip/mL viral stock) resulted in a significant increase (p<0.01) of orofacial pain behavior relative to controls (saline or gfp injection) as assessed by grooming activity at 2 and 6 weeks post injection. Deletion of the IL1R1 gene in these mice abolished their painful behavior.

FIG. 14A shows construction of FIV(IL1ra) expressing IL-1 receptor antagonist. FIG. 14B shows confirmation of vector sequence by multiple restriction enzyme digestions. FIG. 14C shows treatment of 293FT cells with FIV(IL1ra) resulted in induction of IL1ra mRNA as assessed by RT-PCR, (D) which also yielded high levels of IL1ra protein in the supernatant media.

FIG. 15 shows human μ-opioid receptor (HuMOR) is expressed in mammalian cell lines. (A) The HuMOR cDNA was cloned into the multiple cloning site of the pRc/CMV expression vector. Subsequently, the CMV promoter was replaced by the rat neuron specific enolase promoter in the same vector. (B) The CMV-HuMOR was successfully expressed following transient transfection in the neuronal N2α and 293H cell lines, and the NSE-HuMOR gene was expressed in the N2α cell line. Gene expression was detected at the transcript level by RT-PCR, as well as (C) at the protein level by immunocytochemistry in N2α cells transfected with NSE-HuMOR. plain: naïve cells; primers: primers PCR control.

FIG. 16 shows HuMOR lentiviral vectors. (A) The NSE-HuMOR gene was cloned into the Lenti6 viral vector, LV(NSE-HuMOR). (B) VSV-G pseudotyped viral particles (5×106 infectious Particles/mL) were then used to infect N2α cells (m.o.i.˜2) and HuMOR expression was determined at the mRNA level by RT-PCR. Naïve N2α cells as well as cells infected with the LV(lacZ) vector served as controls. (C) LV(HuMOR) was injected intra-particularly in the right and left temporomandibular joints of adult mice. Transduction of trigeminal sensory neurons by HuMOR in the trigeminal ganglia was evaluated by PCR three weeks following viral administration. (D) The HuMOR gene was cloned into the FIV vector downstream to the CMV promoter. (−) Primers only and (+) vector positive PCR controls. CNTL—naïve cells; lacZ, LV(lacZ) infected cells; HuMOR, LV(HuMOR) infected cells; +, positive PCR control; −, primers only PCR control.

FIG. 17 shows FIV(HuMOR) intra-articular injection results in over-expression of μ-opioid receptor in vivo. (A) HuMOR expression was detected at the protein level in the sensory neuron cell bodies by IHC in trigeminal ganglia harvested from mice that received FIV(HuMOR) injections (50 μL containing 5×106 infectious particles) in their temporomandibular joints 9 weeks following intra-articular administration. (B) Conversely, control mice lacked HuMOR immunopositive cells in comparable histology sections. (C) HuMOR expression was detected in proximal nerve fibers located in the brain stem of FIV(HuMOR)-injected mice at the level of the nucleus caudalis, as well as (D) in hard and soft articular tissues of the temporomandibular joint. In particular, HuMOR immunostaining was observed in the articular disc and chondrocytes of the glenoid fossa (temporal bone) as well as of the condylar head. (E) IHC for Met-enkephalin, the endogenous ligand for μ-opioid, showed positive fibers at the level of the subnucleus caudalis. (F) Similar staining was also observed for Leu-enkephalin in the subnucleus caudalis. (G) Met-enkephalin expression was also observed in the synovium located at the posterior aspect of the TMJ. Moreover, the afferent nerve pathways from the temporomandibular joint to the brain stem were traced in a retrograde manner by implanting the DiI compound intra-articularly. (H) DiI immunofluorescence was observed in the main sensory nucleus, as well as (I) in the subnucleus caudalis. (J) There was lack of immunofluorescence in the brain stem of control mice (no DiI). f=glenoid fossa; d=articular disc; c=condylar head. Bar=25

FIG. 18 shows FIV(HuMOR) ameliorates orofacial pain and decreases TMJ pathology. Adult Col1-IL-1β^{XAT} transgenic mice were pre-treated with FIV(HuMOR) intra-articular TMJ injection (50 μL containing 5×106 infectious particles) one week prior to being activated with Cre recombinase. (A) FIV(Cre) bilateral TMJ injection resulted in significant increase of orofacial grooming, a measure of pain, compared to transgenic littermates injected with FIV(gfp) or saline over a time period of 8 weeks. Intra-articular pre-treatment with FIV(HuMOR) prevented the induction of orofacial grooming in “activated” adult IL-1β^{XAT} transgenic mice and had no effect in wild type mice. (B) Resistance to mouth opening was employed as a measure of joint dysfunction. HuMOR transduction ameliorated the arthritis-induced TMJ dysfunction in activated adult IL-1β^{XAT} transgenic mice. (C) Articular pathology was significantly reduced in the FIV(HuMOR) pre-treated Col1-IL-1β^{XAT} mice compared to mice without HuMOR therapy. (D) Moreover, cloning of articular chondrocytes was significantly attenuated in the FIV(HuMOR) pre-treated mice compared to the Col1-IL-1β^{XAT}+Cre mice. (E) Representative TMJ section harvested from Col1-IL-1β^{XAT} mice pretreated with FIV(HuMOR) that was stained with Alcian blue—Orange G histochemistry. (F) Greater magnification of panel E. (G) Representative section of wild type mouse treated with FIV(HuMOR), which had no detectable effects in wild type joints. Bar=25 μm. A total of 5 Tg+Cre, 5 Tg+HuMOR+Cre and 4 WT+HuMOR mice were analyzed. *p<0.05; **p<0.01.

FIG. 19 shows FIV(HuMOR) pre-treatment of the TMJ attenuated arthritis-induced neuronal activation in the trigeminal nuclear complex. c-Fos expression was employed as a marker of neuronal activation in the trigeminal nuclear complex in the brain stem of adult IL-1β^{XAT} transgenic mice. (A) Positive c-Fos IHC was observed in the main sensory nucleus of “activated” transgenic mice, which (B) was attenuated in mice pretreated with FIV(HuMOR). (C) Conversely, littermate controls (Tg+gfp) did not display c-Fos expression in the main sensory nucleus. (D) c-Fos was also induced in the subnucleus caudalis of Tg+Cre mice, primarily observed in neurons, which was ameliorated (E) by HUMOR pretreatment. (F) Control mice did not display c-Fos expression in the subnucleus caudalis. In addition, (G) murine IL-1β was employed as a marker of hyperalgesic neurotransmission. Cytokine expression was increased in the nucleus proprius of arthritic mice (Tg+Cre), which was (H) attenuated in HuMOR-pretreated arthritic mice. (I) Littermate controls displayed minimal expression of mIL1β in these areas. (J) mIL-1β was also induced in the subnucleus caudalis of arthritic mice, and (K) was attenuated by HuMOR pre-treatment. (L) Littermate controls (Tg+gfp) displayed traces of mIL-1β expression. Bar=25 μm. Panels M and N represent quantification of c-Fos and IL-1β positive cells, respectively, in a total of 5 Tg+Cre, 5Tg+HuMOR+Cre and 4 Tg+gfp mice. *p<0.05; **p<0.01.

FIG. 20 shows Joint arthritis induces astroglia activation, which is ameliorated by μ-opioid receptor-mediated anti-nociception. Glial fibrillary acidic protein (GFAP) IHC was employed in the evaluation of astroglia activation in the brain stem of adult Col1-IL-1β^{XAT} mice following bilateral intra-articular FIV(Cre) injections in the temporomandibular joints. (A) FIV(gfp)-transduced adult transgenic mice displayed minimal GFAP expression in the main sensory trigeminal nucleus, whereas (B) FIV(Cre) activation resulted in induction of GFAP immunoreactivity. (C) FIV(HuMOR)-pretreatment ameliorated this GFAP induction in “activated” mice, whereas it had (D) no effect in wild type mice. Similarly, (E) GFAP expression in the subnucleus caudalis was minimal in Tg+gfp mice compared to (F) Tg+Cre littermates. (G) FIV(HuMOR)-pretreatment of the TMJ attenuated the aforementioned arthritis-induced GFAP immunoreactivity in the subnucleus caudalis, whereas (H) had no effect in wild type mice. (I) Quantification of GFAP expression was carried out on tissue sections as described in Methods, with staining intensity set at 100 for wild type tissues in a total of 5 Tg+Cre, 5Tg+HuMOR+Cre and 4 Tg+gfp mice *p<0.05. Bar=25 μm.

FIG. 21 shows intra-cisternal injection of FIV(IL1ra) in Col1-IL1β^{XAT} mice suffering from TMJ arthritis ameliorated orofacial nociceptive behavior.

FIG. 22 shows brainstem neuroinflammation affects TMJ pathology. FIG. 22A shows alcian blue histochemistry (AB/OG), MMP-9 immunohistochemistry (MMP-9), acidic proteoglycans (SO/FG), and type II collagen immunohistochemistry (Col-2) were employed in the histopathological evaluation of the TMJ in the following mouse groups: Control—GFAP-IL1β^{XAT} Tg mice injected with FIV(gfp) in the cisterna magna (brain stem); Experimental—GFAP-IL1β^{XAT} Tg mice injected with FIV(Cre) in the cisterna magna; IL1R1^{-/-}-GFAP-IL1β^{XAT}; IL1RI^{-/-} compound mice injected with FIV(Cre) in the cisterna magna; and FIV(IL1ra)—Col1-IL1β^{XAT} Tg mice that were injected with FIV(Cre) in the TMJ and followed with FIV(IL1ra) injection into the cisterna magna.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method

1. Cross-Talk

As disclosed herein, there is cross-talk between the brain and the periphery when inflammation is present. As used herein, “cross-talk” can refer to the ability of cells in the brain to affect cells in the periphery and the ability of cells in the periphery to affect cells in the brain, for example. The periphery and the central nervous system can communicate in ways that exacerbate the inflammation through a cycle that includes the periphery and the central nervous system. The inflammation can occur both in brain and central neural tissue as well as in the periphery. As disclosed herein, the events at the periphery can affect states in the central nervous system and events in the central nerverous system can affect states in the periphery. This communication occurs through the action of inflammatory mediators (cytokines) which can be either carried in the blood, or directly elaborated by nerve cells. As disclosed herein, sustained peripheral inflammation, such as arthritis of a joint, can lead to inflammation of the central nervous system and subsequent damage to the brain, as in Alzheimer's disease (neurodegeneration) or chronic pain. Furthermore, inflammatory conditions that originate in the brain can affect peripheral tissues during development or adult life, potentially leading to skeletal malformations and degenerative disorders, respectively.

a) Nervous System

In one aspect, the herein disclosed cross-link involves the nervous system. The nervous system can be divided into two parts: central and peripheral. The central nervous system consists of the encephalon or brain and the medulla spinalis or spinal cord. These two parts, the brain and the spinal cord are continuous with on another at the level of the upper border of the atlas vertebra. The peripheral nervous system consists of a series of nerves, which connect the central nervous system to all of the tissues in the body. Nerves also are often grouped as cerebrospinal and sympathetic. However, since the two groups are intimately connected and closely intermingled these distinctions are not absolute. Nerve cells can also be classified as efferent or afferent nerves. Efferent nerve cells are nerve cells that transmit signals from the brain to the periphery and afferent nerve cells are nerve cells that transmit signals from the periphery to the brain.

Neurons act as pain pathways and these pathways include peripheral, spinal, and supraspinal elements. The peripheral part of the system includes the primary afferent sensory neurons. These neurons are called nociceptors, and can be found throughout the body, such as in the skin, muscle, connective tissue, the cardiac system, and abdominal and thoracic viscera. Nociceptors are uncapsulated nerve endings that detect thermal, mechanical, or chemical stimuli, and are thus, not small molecule receptors. Nociceptors can be thinly myelinated or unmyelinated nerve fibers. The thinly myelinated variety are termed A-delta fibers and the unmyelinated variety are termed C-polymodal fibers. The primary functional difference between A and C delta fibers is that A-delta fibers are rapidly conducting and C delta fibers are slowly conducting. This means that A delta fibers transmit sensations perceived as fast, sharp, well-localized pricking pain, and C-polymodal fibers transmit feeling via thermal, mechanical, and chemical stimuli transmitting sensations perceived as dull, aching, burning, poorly localized pain.

Most A-delta and the C-polymodal afferent fibers enter the dorsal horn of the spinal cord by way of the dorsal nerve roots and their ganglia. Wide dynamic range neurons receive nociceptive and non-nociceptive input from the skin, muscle, and viscera. This convergence can account for visceral referred pain. Impulses are then transmitted to the brain by the spinal thalamic tract (STT). Near the thalamus, the STT bifurcates into the neospinothalamic tract and the paleospinothalamic tract, projecting to the thalamus, hypothalamus, periaqueductal gray matter (PAG) in the brain stem. The thalamus processes sensory input is projected to the cerebral cortex, basal ganglia, and limbic system. Descending pathways conduct transmission from the brain to the spinal cord control and modify afferent sensory input.

Nociception can be thought of as the detection of tissue damage by nociceptors. Modulation of nociception occurs peripherally, spinally, and supraspinally. Tissue damage is associated with the release of chemical mediators, such as serotonin, histamine, bradykinin, cytokines, prostaglandins, and leukotrienes, which produce inflammation, and occurs in the peripheral system. The pain transmission is modulated by these events and this lowers excitability threshold of the nociceptor threshold so that stimuli normally non-painful stimuli become painful. This is called nociceptor sensitization. Two other substances that sensitize nociceptors are substance P and glutamate, which can be released from nerve terminals.

The signals from the nociceptors are processed in the dorsal horn of the spine. Repetitive, convergent input from A-delta and C polymodal fibers at the dorsal horn can result in a state where less stimulation is required for the generation of a pain response. This is known as the wind-up phenomenon, and is thought to be initiated by the release of substance P and the excitatory amino acids glutamate and aspartate.

The brain also signals the spinal cord to modulate the pain response. The PAG region of the brainstem contains high concentrations of opioid receptors, and sends projections to the rostral medulla and eventually to the dorsal root inhibiting ascending pain impulses. Thus, the activation of the opioid receptors interrupts the transmission of the pain signal. Descending pathways can also stimulate spinal nociceptive transmission as well.

b) Glial Activation

As disclosed herein, chronic peripheral inflammation leads, in addition to the development of pain, to glial cell activation at the dorsal horns of the spinal cord following sustained excitation of primary (1°) sensory afferent fibers. To this end, excitatory neurotransmitters such as glutamate and substance P (SP) mediate this neuron to glia signaling. In turn, activated glial cells, through the expression of various inflammatory mediators, such as inflammatory cytokines and prostanoids, such as IL-1β, can induce localized neuroinflammation at the dorsal horn proximal to the region exhibiting sensory input. As disclosed herein, glial activation and the subsequent development of neuroinflammation at the level of the dorsal horns plays an important role in the processing of peripheral inflammatory pain. Specifically, glia-derived neuroinflammation can influence the central processing of pain by inducing excitation in post-synaptic neurons. Furthermore, pre-synaptic (1° order) neurons can also be affected by this mechanism resulting in further excitation of the primary afferent sensory fibers.

Disclosed herein is the role of glial cells in pain and the mechanism by which localized neuroinflammation at the level of the medullary dorsal horn (brain stem) can influence pain processing. Thus, provided herein are compositions and methods for treating peripheral inflammation in a subject, comprising administering to the central nervous system of the subject a modulator of inflammation. In certain embodiments, this administration can be directly at the brain stem rather than a systemic or periphereal administration. For example, the modulator of inflammation can be administered directly to the dorsal horn, cisterna magna, or thecal sac.

Also as disclosed herein, glial activation resulting from peripheral inflammation can lead to neuroinflammatory disease. Thus, treatment of peripheral inflammation can treat or prevent neuroinflammatory disorders. Thus, provided herein are compositions and methods for treating a brain disorder in a subject, comprising administering a modulator of inflammation to a site of peripheral inflammation in the subject.

A further advantage of the provided compositions and methods relates to the reciprocal relationship between the nervous system and bones/joints, wherein neuroinflammation will affect bone development (osteoporosis, arthritis, etc.), and bone/joint disease can influence neuralogical function. For example, normal craniofacial growth is dependant at least in part on the physiologic function of the sympathetic nervous system via post-ganglionic sympathetic fibers innervating the synchodroses of the cranial base. Altered sympathetic nervous system impact skeletal pattern formation and cartilage maturation with alteration of catecholamine homeostasis as the bridge connecting the two systems. Thus, provided herein are compositions and methods for treating or preventing bone disease in a subject, comprising administering a modulator of inflammation to the central nervous system of the subject. For example, the modulator of inflammation can be administered directly to the dorsal horn, cisterna magna, or thecal sac.

Also provided are compositions and methods for the treatment of subjects with brain disorders using bone/joint treatments known in the art, such as, for example, parathryroid hormone (PTH). Further provided are compositions and methods for the treatment of subjects with brain disorders with anti-inflammatories, e.g. FIV(IL1-ra), that can prevent/reduce bone diseases. Further provided are compositions and methods for the treatment of subjects with joint diseases, wherein said treatment can also attenuate neurological disease.

In one aspect, the modulator of inflammation for the provided methods can modulate the pro-inflammatory cytokine interleukin-1β via paracrine and/or endocrine pathways. For example, the modulator of inflammation for the provided methods can be interleukin-1 receptor antagonist factor (IL-1ra). Alternatively, the modulator of inflammation for the provided methods can be IL-1β. Further, the modulator of inflammation for the provided methods can be a cell, such as a myeloblastoid immune cell (e.g., monocyte, macrophage, dendritic cell, or a precursor thereof), expressing the diffusible IL-1ra or IL-1β.

“Modulate” or “modulating” refers to an increase or decrease in an activity. This can include but is not limited to the inhibition or promotion of an activity, condition, disease, or response or other biological parameter. Whether an inhibitor or activator of inflammation is preferred can depend on the site and stage of inflammation. For example, the disclosed modulator of IL-1β can be an inhibitor or an activator of IL-1β signaling. It is within one of skill in the art to use there herein disclosed methods and models to determine the preferred modulation based on the site and stage of inflammation. Thus, as used herein, “inhibit” or “inhibitor” can also refer to modulators such as activators and inducers unless expressly stated to the contrary.

2. Inflammation

The herein disclosed cross-link can involve localized neuroinflammation at the dorsal horn proximal to the region exhibiting sensory input. Thus, compositions and methods that modulate inflammation in the central neural tissue (e.g., glial cells of the dorsal horn) can have an effect on distal sites of inflammation and pain. Likewise, compositions and methods that modulate peripheral inflammation can affect neuroinflammation and disorders resulting therefrom.

Thus, provided herein are compositions, including polypeptides, nucleic acids, vectors, and cells, that can be used to modulate inflammation. Inflammation is a localized protective reaction of tissue to irritation, injury, or infection, characterized by pain, redness, swelling, and sometimes loss of function. As used herein, “inflammatory disorder” or “inflammatory disease” refers to any condition, disease or disorder wherein inflammation is involved, such as the sustained or chronic inflammation that occurs when tissues are injured by viruses, bacteria, trauma, chemicals, heat, cold or any other harmful stimulus. Irritation or discomfort can result from inflammation in a mammal due to, for example, skin inflammation, eye inflammation, gut inflammation or the like.

In one aspect, the peripheral inflammation of the disclosed methods is arthritis. Arthritis as a disease can include many different disorders and symptoms and can affect many parts of the body. Arthritis typically causes pain, loss of movement and sometimes swelling. Arthritis is actually a term used for a set of more than 100 current medical conditions. Arthritis is most commonly associated with older individuals, but can start as early as infancy. Some forms affect people in their young-adult years. A common aspect among arthritic conditions is that they affect the musculoskeletal system and specifically the joints—where two or more bones meet. Arthritis-related joint problems can include pain, stiffness, inflammation and damage to joint cartilage (the tough, smooth tissue that covers the ends of the bones, enabling them to glide against one another) and surrounding structures. Such damage can lead to joint weakness, instability and visible deformities depending on the location of joint involvement. Many of the arthritic conditions are systemic, in that they affect the whole body. In these diseases, arthritis can cause damage to virtually any bodily organ or system, including the heart, lungs, kidneys, blood vessels and skin.

Some different types of arthritis are osteoarthritis, rheumatoid arthritis, gout, ankylosing spondylitis, juvenile arthritis, systemic lupus erythematosus (lupus), scleroderma, and fibromyalgia. Osteoarthritis is a degenerative joint disease in which the cartilage that covers the ends of bones in the joint deteriorates, causing pain and loss of movement as bone begins to rub against bone. It is the most prevalent form of arthritis. Rheumatoid arthritis is an autoimmune disease in which the joint lining becomes inflamed as part of the body's immune system activity. Rheumatoid arthritis is one of the most serious and disabling types, affecting mostly women. Gout affects mostly men. It is usually the result of a defect in body chemistry. This painful condition most often attacks small joints, especially the big toe. Fortunately, gout almost always can be completely controlled with medication and changes in diet. Ankylosing spondylitis is a type of arthritis that affects the spine. As a result of inflammation, the bones of the spine grow together. Juvenile arthritis is a general term for all types of arthritis that occur in children. Children can develop juvenile rheumatoid arthritis or childhood forms of lupus, ankylosing spondylitis or other types of arthritis. Systemic lupus erythematosus (lupus) is a disorder that can inflame and damage joints and other connective tissues throughout the body. Scleroderma is a disease of the body's connective tissue that causes a thickening and hardening of the skin. Fibromyalgia is a disorder in which widespread pain affects the muscles and attachments to the bone. It affects mostly women.

Neuroinflammation, characterized by activated microglia and astrocytes and local expression of a wide range of inflammatory mediators, is a fundamental reaction to brain injury, whether by trauma, stroke, infection, or neurodegeneration. This local tissue response is surely part of a repair and restorative process. Yet, like many inflammatory conditions in peripheral diseases, neuroinflammation can contribute to the pathophysiology of CNS disorders. For example, in Alzheimer's disease (AD), glial-driven inflammatory responses to Aβ deposition are thought to promote neurodegeneration, as evidenced by the extent of neuroinflammation in AD, increased risk for AD with certain polymorphisms of proinflammatory cytokine genes, and reduction in disease risk for individuals taking nonsteroidal anti-inflammatory drugs (NSAIDs).

Considered herein is the use of the provided compositions and methods relate to the study and treatment of any inflammatory disease. Thus, the provided compositions and methods relate to the study and treatment of inflammatory bowel disease. The provided compositions and methods relate to the study and treatment of chronic dermatological disorders.

A particular advantage of the provided compositions and methods is the herein described ability to deliver inflammatory mediators, and the disclosed modulators thereof, to the brain by means of peripheral administration. For example, FIV vectors are disclosed herein that can deliver the herein disclosed nucleic acids to target sites within the subject. The disclosed FIV constructs can be delivered systemically by injection into the circulation or locally by injection into the target site, such that either method of administration can result in the delivery of the nucleic acid to cells in the brain, such as, for example, microglia or astrocytes. The use of FIV vectors to deliver nucleic acids or transgenes to the brain following systemic administration is described in U.S. patent application Ser. No. 10/978,927 and Patent Cooperation Treaty Application No. PCT/US05/04885, which are herein incorporated by reference in their entirety as they related to this teaching Thus, neural inflammatory disorders, as disclosed herein, can be treated through delivery of an inflammatory mediator, as discussed herein, via, for example, injection in the joint of the subject. In addition, inflammatory conditions related to bone and/or joints can be treated by injection into the joint or through system injection or IP injection as discussed herein.

Chronic inflammatory disorders includes arthritis, inflammatory bowel disease, chronic obstructive pulmonary disease, psoriasis and atherosclerosis—all with large markets. Twelve percent of adults have osteoarthritis and in the US, clinical osteoarthritis is diagnosed in 21 million patients and is the cause of nearly 500,000 hip and knee replacement surgeries. Another 2 million patients have rheumatoid arthritis.

3. Pain

Prolonged damage to tissues, i.e., resulting from inflammation, will eventually result in plastic (non reversible) changes in the neurons that process pain from that area, which now facilitate either allodynia and/or hyperalgesia. Chronic pain is born following these plastic neuronal changes, whereby the neurons are now “sick” and pain will occur even in the absence of peripheral stimulus (e.g., amputated limbs, extracted teeth). In fact, its basis is neuropathic now, and neurons continuously send pain messages to the brain even though there is no continuing tissue damage. Thus, chronic pain can be treated or prevented by inhibiting the chronic inclammation resulting from the reciprocal cross-talk between the periphery and the central neural tissue. Another advantage of the disclosed cross-talk between the periphery and the central nervous system is the ability to treat chronic pain and peripheral inflammatory disorders by inhibiting pain impulses within the central neural tissue, e.g., dorsal horn.

About one and a half billion people suffer from moderate to severe chronic pain worldwide and approximately 50 million Americans suffer with pain. Pain is typically classified into two categories: nociceptive pain (somatic pain) and neuropathic pain. Nociceptive pain is pain that is sensed after some type of trauma. The nociceptive pain is sensed by the “nociceptor” sensory fibers which are connected to the nervous system. After an injury to a muscle, soft tissue (ligaments, tendons), bones, joints, or skin (or other organs), these sensory fibers are stimulated which causes a transmission of a signal through an afferent neuron to the brain. Nociceptive pain is often characterized as a deep aching, throbbing, gnawing, or sore sensation. Common examples of nociceptive pain include: pain after trauma (e.g. a car accident or a fall), postoperative pain, and arthritis pain. Nociceptive pain is usually localized and gets better with healing.

Neuropathic pain is pain caused by damage to nerve tissue. Neuropathic pain is often characterized as burning, severe shooting pains, and/or persistent numbness or tingling. Common examples of neuropathic pain related to back pain include sciatica, pain that travels from the spine down the arm, and pain that persists after back surgery.

It is thought that in some cases prolonged nociceptive pain may progress to neuropathic pain, and a patient may have both nociceptive and neuropathic pain at the same time. Pain is also often classified as acute pain or chronic pain. Acute pain is characterized as pain where the amount of pain directly correlates with the level and duration of tissue damage. Acute pain therefore, provides a protective reflex, such as the reflex to move your hand immediately if you touch a sharp object. This type of pain is a symptom of injured or diseased tissue, so that when the underlying problem is cured the pain goes away. Acute pain is a form of nociceptive pain. Chronic pain on the other hand, does not correlate with the severity of the insult, and therefore, typically will not serve a protective function. Prolonged damage to tissues, i.e. knee pain or tooth ache, will eventually result in plastic (non reversible) changes in the neurons that process pain from that area, which now facilitate either allodynia and/or hyperalgesia. Chronic pain is born following these plastic neuronal changes, whereby the neurons are now “sick” and pain will occur even in the absence of peripheral stimulus (e.g., amputated limbs, extracted teeth). In fact, its basis is neuropathic now, and neurons continuously send pain messages to the brain even though there is no continuing tissue damage. Neuropathic pain is a form of chronic pain. Disclosed herein are methods and mechanisms and compositions that treat and reduce chronic pain. The mechanism that causes chronic pain is disclosed and its relationship between periphery nerve signaling and dorsal nerve signaling and inflammation are disclosed.

a) Management of Pain

The herein provided compositions and methods can further comprise the use of pain management compositions and methods. Non-steroidal anti-inflammatory drugs (NSAID's) are often utilized as the first line of agents for the management of pain. NSAID's primarily exert their pain-killing effects by inhibiting the production of prostanoids and attenuating peripheral inflammatory conditions that may be responsible for pain elicitation. Alternatively, corticosteroids may be utilized with peripheral routes of action. In contrast, exogenously administered opioid drugs (morphine) mimic the effects of the endogenous opioids by crossing the blood brain barrier (BBB). Similarly, tricyclic antidepressants that cross the BBB have been also employed in cases of chronic pain by inhibiting the reuptake of serotonin and norepinephrine. However, each of these four classes of drugs is characterized by significant side effects that prohibit their long term use as well as often show unfavorable treatment outcomes.

b) Opioid Receptors and Mechanism of Action

Opioid analgesics have been used for pain management for hundreds of years. Opium itself consists of the dried latex from the unripe fruit of the opium poppy Papaver somniferum. Morphine is isolated from opium. Opioid receptors exist in the spinal and supraspinal regions of the nervous systems. Opioids can modulate neuronal transmission by binding to opioid receptors in the dorsal-root ganglia, the central terminals of primary afferent neurons (LaMotte C, et al., Brain Res 1976; 112:407-12; Fields H L, et al., Nature 1980; 284:351-3) and peripheral sensory-nerve fibers and their terminals (Stein C, et al., Proc Natl Acad Sci USA 1990; 87:5935-9; Hassan A H S, et al.,. Neuroscience 1993; 55:185-95. The dorsal-root ganglia and trigeminal ganglion (Xie G X, et al., Life Sciences 1999; 64:2029-37; Li J L, et al., Brain Res 1998; 794:347-52.) contain messenger RNA (mRNA) for opioid receptors (Schafer M, et al., Eur J Pharmacol 1995; 279:165-9; Mansour A, et al., Brain Res 1994; 643:245-65) and primary afferent nerves mediate the peripheral antinociceptive effects of morphine (Bartho L, et al., Naunyn Schmiedebergs Arch Pharmacol 1990; 342:666-70). The presence of endogenous opioids and their receptors are able to produce a potent anti-nociception. Opioids increase potassium currents and decrease calcium currents in the cell bodies of sensory neurons (Werz M A, Macdonald R L., Neurosci Lett 1983; 42:173-8; Schroeder J E, et al., Neuron 1991; 6:13-20), both of which can lead to the inhibition of neuronal firing and transmitter release. A similar process occurring throughout the neuron, can explain why opioids attenuate both the excitability of the peripheral nociceptive terminals and the propagation of action potentials (Andreev N, et al., Neuroscience 1994; 58:793-8; Russell N J W, et al., Neurosci Lett 1987; 76:107-12). At central nerve terminals, (Yaksh T L, et al., Nature 1980; 286:155-7) opioids inhibit the calcium-dependent release of excitatory, pro-inflammatory compounds (e.g., substance P) from peripheral sensory-nerve endings, (Yaksh T L., Brain Res 1988 458:319-24) which contribute to the anti-inflammatory actions of opioids (Barber A, Gottschlich R. et al., Med Res Rev 1992; 12:525-62).

There are three known opioid receptors, μ, κ, and δ-opioid receptors. μ-Receptors are further subdivided into at least two subclasses, μ1 and μ2-receptors. The body produces opioid like molecules, called endogenous opioids, such as endorphins, enkephalins, and dynorphins. μ-receptors are known to mediate analgesia, respiratory depression, bradycardia, nausea/vomiting, and decreased gastrointestinal tone.

δ-receptors mediate supraspinal analgesia, and κ-receptors mediate dysphoria and tachycardia. As pain impulses ascend through the spinal and supraspinal nervous system, activation of the opioid receptors inhibits these impulses and inhibits the transmission of pain from the dorsal horn. In addition, opioid analgesics prevent the presynaptic release of pain mediators such as Substance P into the spinal cord region.

All animals, such as mammals, such as human, contain opioid receptors. It is understood that there are homologs for the various opioid receptors across animal species. A number of different opioid receptor sequences are set forth in the SEQ IDs, including μ-opioid receptors. The human μ-opioid receptor is referred to herein as HUMOR. It is understood that if a particular statement or reference is made regarding HUMOR that this statement is equally applicable to homologous receptors, unless specifically indicated otherwise.

Opioid analgesics are typically grouped into three classes: naturally occurring (morphine, codeine); semi-synthetic morphine derivatives (hydromorphone, oxycodone, hydrocodone); and synthetic. Synthetic opioid analgesics include morphinan derivatives (levorphanol); methadone derivatives (methadone, propoxyphene); benzomorphan derivatives (pentazocine); and phenylpiperidine derivatives (meperidine, fentanyl, sufentanil, alfentanil, remifentanil). Tramadol is an opioid analgesic that also inhibits the reabsorption of norepinephrine and serotonin.

Another way to classify opioid analgesics is as agonists, partial agonists, mixed agonists/antagonists, and antagonists based on their interactions at the opioid receptors. μ and κ opioid-receptors are stimulated by agonists. Partial agonists have reduced μ-opioid receptor activity, and mixed agonists/antagonists only stimulate certain μ and κ-opioid receptors. Antagonists bind μ and κ-opioid receptors but do not stimulate the receptor activity.

Some agonists are Morphine, Hydromorphone, Oxymorphine, Codeine, Oxycodone, Hydrocodone, Dihydrocodeine, Methadone, Meperidine, Fentanyl, Sufentanil, Alfentanil, and Remifentanil. An example of a partial agonist is Buprenorphine. Pentazocine, Nalbuphine, and Butorphanol are examples of mixed agonists/antagonists. Examples of antagonists are Naloxone and Nalmefene. It is understood that one way to classify opioid receptors is by which molecules act as antagonists and which act as agonists, for example. Thus, a receptor can be defined as “a receptor for which morphine is an agonist.”

There are a number of adverse side effects that can occur when taking opioid analgesics, such as CNS effects, such as sedation, confusion, and respiratory depression. Gastrointestinal adverse effects include nausea, vomiting, and constipation. Involuntary muscular contractions (twitching) known as myoclonus, bradycardia, and hypotension, can also occur. Lastly, physical and psychological dependence can also occur upon use or prolonged use of opioid analgesics. Thus there is a significant need for the disclosed compositions and methods, which reduce or eliminate the need for opioid analgesics in many indications.

c) μ-Opioid Receptor Therapy for Pain

The management of pain using the targeted expression of opioid receptor(s), such as the μ-opioid receptor, is described in U.S. patent application Ser. No. 10/546,179, which is herein incorporated by reference in its entirety for this teaching. The disclosed approach for the management of pain involves the targeted expression of opioid receptor(s) such as the μ-opioid receptor in the primary neurons innervating the areas, such as orofacial areas, experiencing pain, resulting in these same neurons becoming more susceptible to the intrinsic opioid anti-nociceptive mechanisms. Thus, disclosed are compositions and methods for treating pain. The compositions comprise an opioid receptor that is expressed from a vector. Typically these compositions will be delivered to at the point of pain It is thought that their expression, increases the efficiency of the body's own opioid like molecules and decreases pain.

As disclosed herein, the cDNA for a human μ-opioid receptor (HUMOR) is set forth in SEQ ID NO:93. The μ-opioid receptor (Raynor K, et al., J Pharmacol Exp Ther. 1995; 272:423-8) has been placed into a vector herein and expressed in primary fibroblasts as well as cells of the N2a neuronal cell line. Transduction and stable expression of μ-opioid receptor in neurons can be accomplished by employing VSV-G pseudotyped immunodeficiency viral vectors (FIV).

The expression of the μ-opioid receptor in the neurons at the point of pain in certain embodiments requires transduction in a non-dividing cell such as a neuron. This can be accomplished using a transduction mechanism, such as lipofection or encapsulation methods, or via viral vector systems that function with cell division, such a lentiviruses, such as the FIV virus, or adeno-associated viruses, rAAV vectors, HSV Amplicon, and liposomes.

It has been previously shown that this FIV system is capable, due to its lentiviral properties, of infecting terminally differentiated cells, including neurons. Disclosed are methods for administering vectors, such as the FIV(μ-opioid receptor) vector, peripherally at the site of pain. The neurons innervating that specific site and mediating the nociceptive signals are infected and stably transduced. These vectors, including vectors expressing lacZ and the μ-opioid receptor, can transduce nerve cells in vivo, in mice, through injection at the periphery.

Disclosed herein is the stable expression of a reporter gene, the lacZ gene, in neurons located in the appropriate region of the trigeminal ganglion following peripheral injection of FIV(lacZ) in the area of the TMJ, as well as a variety of expression vectors containing the μ-opioid receptor, such as the human μ-opioid receptor.

Disclosed are vectors wherein the vector includes sequence encoding the μ-opioid receptor gene. Also disclosed are vectors, wherein a μ-opioid receptor gene has been cloned in an FIV vector. Disclosed are methods comprising administering the disclosed vectors to cells, including cells involved in transmitting pain signals, such as nerve cells in the orofacial regions, related to for example, pain from TMJ and the masseter muscle.

Also disclosed are transgenic mice that have been stably transfected with the disclosed vectors. These mice can be used, for example, as models of pain and the testing of therapeutics.

d) μ-Opioid Receptor Therapy for Inflammation

Compositions comprising opioid receptors such as HuMOR, in addition to reducing pain, can also reduce peripheral inflammation, such as arthritis. This effect can be either indirect or direct. For example, the alleviation of nociception by HuMOR following transduction of neurons can lead to a reduction in inflammation. Alternatively, transduction of joint tissues by compositions comprising HuMOR can directly reduce peripheral inflammation. For example, the over-expression of HuMOR in chondrocytes of the joint can have an anti-inflammatory effect.

Compositions comprising opioid receptors such as HuMOR, in addition to reducing pain, can also reduce neural inflammation. Thus, the opioid opioid receptors such as HuMOR can be inflammatory mediators as disclosed and used herein. Thus, HuMOR can be administered to the central nervous system for the treatment of peripheral inflammation due to a reduction or inhibition of central inflammation.

4. Chondrocyte Maturation

A further advantage of the provided compositions and methods relates to the reciprocal relationship between the nervous system and bones/joints, wherein neuroinflammation will affect bone development (osteoporosis, arthritis, etc.), and bone/joint disease can influence neurological function. For example, normal craniofacial growth is dependant at least in part on the physiologic function of the sympathetic nervous system via post-ganglionic sympathetic fibers innervating the synchodroses of the cranial base. Altered sympathetic nervous system impact skeletal pattern formation and cartilage maturation with alteration of catecholamine homeostasis as the bridge connecting the two systems. Thus, provided herein are compositions and methods for treating or preventing bone disease in a subject, comprising administering a mediator of inflammation to the central nervous system of the subject.

As disclosed herein, targeted deletion of the murine HexB locus impairs chondrocyte maturation at the long bone growth plates as well as cranial base synchondroses, ultimately affecting skeletal growth and development. The resulting HexB^{-/-} skeletal anomalies mice can be rescued following systemic neonatal restitution of β-hexosaminidase, indicating that β-hexosaminidase deficiency impacts chondrocyte differentiation and maturation during late embryonic or early postnatal (perinatal) stages of development.

The lack of β-hexosaminidase expression in chondrocytes together with the transduction of liver cells following neonatal FIV(HEX) systemic administration (Kyrkanides, S., et al. 2005) indicate that the corresponding skeletal amelioration is mediated by an endocrine pathway of cross-correction. Receptor-mediated enzyme transfer (cross-correction) is an important characteristic of lysosomal enzymes, including β-hexosaminidase, whereby the secreted enzyme can be up-taken by neighboring cells (paracrine pathway) or through the blood circulation at distal locations (endocrine pathway). The transport and compartmentalization of soluble lysosomal enzymes to lysosomes depends on the recognition of mannose 6-phosphate (Man-6-P) residues in their oligosaccharide moiety by specific receptors. Two distinct proteins have been thus far identified capable of interacting with lysosomal enzymes, the Man-6-P receptor (MPR; 270 kDa) which also binds the insulin-like growth factor-II (IGF-II), and the cation-dependent MPR (CD-MPR; 46 kDa) (Munier-Lehmann, H, et al. 1996). Cross-correction based treatments, such as enzyme replacement therapy (ERT) and bone marrow transplantation (BMT) have successfully addressed peripheral enzymatic deficiencies in the past (von Specht, B. U., et al. 1979).

A number of growth factors regulate chondrogenesis and chondrocyte maturation, with PTHrP representing a central regulator. Specifically, PTHrP acts both as an inducer of chondrogenic commitment (de Crombrugghe, B., et al. 2000) and as an inhibitor of chondrocyte hypertrophic progression (Ionescu, A. M., et al. 2001). The critical regulatory role of PTHrP in these processes is best exemplified in genetically altered mice where either PTHrP has been ablated or a constitutively activated mutant of its receptor has been over-expressed in cartilage. Any alterations in the maturational program of chondrocytes can disrupt normal growth plate function and result in significant skeletal abnormalities.

Another osteogenesis-associated gene found upregulated in the HexB^{-/-} chondrocytes was COX-2. Several studies in avian mesenchymal limb bud cells suggest an important role for cyclooxygenase during chondrogenesis. Both indomethacin (Chepenik, K. P., et al. 1984; Biddulph, D. M., et al. 2000) and blockade of PGE₂ inhibit chondrogenesis (Biddulph, et al. 1991; Capehart, A. A., & Biddulph, D. M. 1991). Addition of PGE₂ to mesenchymal limb bud cultures (i) enhances chondrogenesis; and (ii) stimulates chondrogenesis in the presence of indomethacin, a COX inhibitor (Kosher, R. A., & Walker, K. H. 1983). Prostaglandins are synthesized by growth plate chondrocytes (Wong, P. Y., et al. 1977) and synthesis is altered by mechanical loading (Mankin, K. P., & Zaleske, D. J. 1998). Systemic injection of PGE₂ results in a thinner growth plate with decreased size of hypertrophic chondrocytes and reduced limb growth (Ueno, K., et al. 1985; Furuta, Y., & Jee, W. S. 1986). In addition, prostaglandins stimulate growth plate chondrocyte proliferation and sulfate incorporation (O'Keefe, R J., et al. 1992) while inhibiting growth plate maturation (Zhang, X., et al. 2004; Li, T. F., et al. 2004). The COX-2 induction in Sandhoff chondrocytes is consistent with modulating development of skeletal dysplasia in these mice. Moreover, COX-2 is consistent with being a node of convergence for a number of genetic defects, whereby activation of the cyclooxygenase-prostanglandin pathway may be responsible in part for the skeletal defects. Hence, timely inhibition of cyclooxygenase activity in affected individuals can manage skeletal dysplasias, such as the use of NSAIDs and COX-2 selective inhibitors.

Thus, there is a phenotypic switch of HexB^{-/-} chondrocytes from a proliferative/pre-hypertrophic phenotype to a hypertrophic/terminally mature type in the long bone growth plates and cranial base synchondroses. The successful neonatal rescue of the Sandhoff skeletal defects indicates perinatal impairment of chondrocyte maturation secondary to β-hexosaminidase deficiency. Further, PGE₂ has stimulatory effects on C₂C₁₂ differentiation from a myoblastic to an osteoblastic phenotype. Taken together, these findings indicate an acceleration of chondrocyte maturation secondary to β-hexosaminidase deficiency during perinatal stages of development.

5. Inflammatory Mediator

Inflammation can be affected in part by modulating the expression or activity of an inflammatory mediator. An inflammatory mediator, as used herein, refers to a protein that modulates inflammation and includes, for example, cytokines (e.g., IL-1β) prostaglandins (e.g., prostaglandin E₂ (PGE₂)), prostaglandin synthases (e.g., COX-1, COX-2, cPGES, and mPGES), and modulators thereof.

a) Interleukin-1

An example of an inflammatory mediator is interleukin-1 (IL-1). IL-1 is a potent immunomodulating cytokine that exists as two principal isoforms, IL-1α and IL-1β. These two molecules show significant divergence in sequence and have somewhat different roles with IL-1α generally thought to be involved in direct cell:cell communication, whereas IL-1β is secreted. Nevertheless, these two molecules act through the same membrane-associated receptor known as IL-1 receptor type 1 (IL-1R1) to promote a proinflammatory signaling cascade that includes the activation of NFκB and MAP kinases [Rothwell, N. J. and G. N. Luheshi. Trends Neurosci. (2000) 23:618-625].

At least two molecules have been identified that antagonize the effects of IL-1. IL-1 receptor antagonist (IL-1ra) competes for receptor binding, and IL-1 receptor type 2 (IL-1R2), which lacks an intracellular domain, is thought to serve as a decoy receptor [Rothwell, N. J. and G. N. Luheshi. Trends Neurosci. (2000) 23:618-625]. Expression of each of these molecules is regulated. The contribution of IL-1 to an inflammatory response therefore depends on the relative balance of expression between these family members [Arend, W. P. Cytokine & Growth Factor Rev. (2002) 13:323-340]. In one example, the mature form of IL-1β is attached to the secretion signal from IL-1ra, which is the same sequence as the secretion signal sequence of IL-1β.

Lavage and explant studies from normal and osteoarthritic cartilage support the contention that cytokines are up regulated in disease states. Specifically, Moos et al. [Moos V, et al. (1999) J Rheumatol 26:870-9] have demonstrated that cartilage from knee or hip joints in 10 patients with osteoarthritis (OA) compared to controls demonstrated cytokines, including IL-1β that are up regulated in OA cartilage. Shin et al. [Shin S-j, et al. (2003) J Appl Physiol.; 95:308-13] examined the influence of mechanical stress on matrix turnover in the meniscus in the presence of IL-1β to determine the role of nitric oxide (NO) in these processes. Stimulation of proteoglycan release in response to compression was dependent on NOS2 regardless of the presence of IL-1. These finding suggest that IL-1 can modulate the effects of mechanical stress on extracellular matrix turnover through a pathway that is dependent on NO. Joosten et al. [Joosten L A, et al (1999) J Immunol; 163:5049-55] have demonstrated that blocking of IL-1 is a cartilage and bone protective therapy in destructive arthritis, whereas the TNF-alpha antagonist has little effect on tissue destruction. Webb et al. [Webb G R, et al. (1998) Osteoarthr & Cartil 6167-76] demonstrated that OA synovium supernatants contained higher concentrations of interleukin-1 beta (IL-1 beta) and interleukin-6 (IL-6) than normal synovial supernatants and neutralizing antibodies to these cytokines either partially or totally abrogated the ability of the OA supernatants to increase chondrocyte p55 TNF-R expression. These results suggest that IL-1 and IL-6 produced by OA synovium contribute to the progression of the disease by rendering chondrocytes more susceptible to stimulation by catabolic cytokines. Smith et al. [Smith M D, et al. (1997) J Rheumatol 24:365-71] examined the production of IL-1α, IL-1β and TNFα in synovial membranes from patients with OA, irrespective of the degree of articular cartilage damage. They suggest that chronic inflammatory changes with production of proinflammatory cytokines are a feature of synovial membranes from patients with early OA, with the most severe changes seen in patients at the time of joint replacement surgery. This low grade synovitis results in the production of cytokines that can contribute to the pathogenesis of OA.

b) Cyclooxygenase COX

Another example of an inflammatory mediator is the enzyme cyclooxygenase (COX). Cyclooxygenase is the principal target of non-steroidal anti-inflammatory drugs (NSAIDs), which are a mainstay of treatment for many inflammatory conditions. Cyclooxygenase catalyzes the first step in the conversion of arachidonic acid to prostanoids, a group of potent lipid mediators acting in diverse physiological processes.

Cyclooxygenase is known to exist in two isoforms: COX-1, which in many tissues appears to be constitutively expressed and responsible for homeostatic production of prostanoids, and COX-2, which is often referred to as the “inducible” isoform since its expression is rapidly modulated in response to diverse stimuli such as growth factors, cytokines, and hormones (O'Banion M K, et al. (1991). J Biol Chem 266: 23261-7; O'Banion M K, et al. (1992). Proc Natl Acad Sci U.S.A. 89:4888-92). The distinction between these two COX isoforms, the roles they play, and the actions of prostanoids have been previously reviewed (Vane J R, et al. (1998). Annu. Rev. Pharmocol. Toxicol. 38:97-120; Smith, W L, et al. (2000). Annu Rev Biochem 69:145-82].

Interest in selectively inhibiting production of PGE₂, the principle inflammatory prostanoid, has been heightened by recognition of at least two PGE₂ synthase isoforms that are reportedly coupled to distinct COX isoforms. More specifically, a membrane-associated isoform (mPGES) is functionally coupled to COX-2, whereas a cytosolic enzyme (cPGES) appears to be linked to a COX-1-dependent production of PGE₂ (Tanioka et al. 2000; Murakami et al., 2000). Although cellular localization can play some role, functional coupling is largely a factor of expression patterns: as with COX-2, mPGES is dramatically upregulated by proinflammatory stimuli, whereas cPGES is constitutively expressed in cell systems examined to date (Jackobson et al., 1999; Stichtenoth et al., 2001; Han et al., 2002). In addition, COX-2 and mPGES are coordinately upregulated in a rat model of adjuvant arthritis (Mancini et al., 2001).

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular xxx is disclosed and discussed and a number of modifications that can be made to a number of molecules including the xxx are discussed, specifically contemplated is each and every combination and permutation of xxx and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Anti-Inflammatory Agents

Anti-inflammatory and/or anti-histaminic agents can be used in the herein disclosed compositions and methods. Non-limiting examples of anti-inflammatory agents include Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Momiflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; and Zomepirac Sodium.

Non limiting examples of anti-histaminic agents include Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine.

2. Modulators of Inflammatory Mediator

Provided herein are compositions that act to modulate an activity of an inflammatory mediator. “Activity,” as used herein, refers to any function or process of a composition disclosed herein and includes, for example, transcription, translation, post-translational modification, translocation, homophilic or heterophilic binding, secretion, endocytosis, or degredation. Disclosed therefore are compositions that inhibit one or more activities of an inflammatory mediator provided herein. These compositions are refered to herein as inflammatory mediator inhibitors. Inhibition or a form of inhibition, such as inhibit or inhibiting, as used herein means to restrain or limit. Reduce or a form of reduce, such as reducing or reduces, as used herein, means to diminish, for example in size or amount. It is understood that wherever one of inhibit or reduce is used, unless explicitly indicated otherwise, the other can also be used. For example, if something is referred to as “inhibited,” it is also considered referred to as “reduced.”

a) Knockdown of Gene Expression

The activity of an inflammatory mediator can be modulated at the gene expression level. The disclosed inflammatory mediator inhibitor can be a gene expression inhibitor. Methods of targeting gene expression are generally based on the sequence of the gene to be targeted. Disclosed are any such methods that can be applied to the targeted knockdown of an inflammatory mediator. By “knockdown” is meant a decrease in detectable mRNA expression. Nucleic acids are generally used to knockdown gene expression and generally comprise a sequence capable of hybridizing to the target sequence, such as mRNA. Examples of such functional nucleic acids include antisense molecules, ribozymes, triplex forming nucleic acids, external guide sequences (EGS), and small interfering RNAs (siRNA).

Antisense molecules are designed to interact with a target nucleic acid molecules through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437. However, the effect of iRNA or siRNA or their use is not limited to any type of mechanism.

Disclosed herein are any antisense molecules designed as described above based on the sequences for the herein disclosed inflammatory mediators. Examples of antisense sequences are disclosed herein for IL-1α (SEQ ID NO:70), IL-1β (SEQ ID NO:71), COX-1 (SEQ ID NO:72), COX-2 (SEQ ID NO:73), cPGES (SEQ ID NO:74), and mPGES (SEQ ID NO:75).

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Disclosed herein are any ribozymes designed as described above based on the sequences for the herein disclosed inflammatory mediators. Hammerhead ribozymes can cleave RNA substrates at for example, a 5′-GUC-3′ sequence, cleaving the mRNA immediately 3′ to the GUC site. Engineered hammerhead ribozymes, which cleave at a different sequence are known and disclosed, for example, in the patents disclosed herein, and are incorporated by reference. A hammerhead ribozyme is typically composed of three parts. The first part is a region which will hybridize to the sequence 5′ of the GUC recognition site, and can be called a first hybridzation arm. A second part consists of a core catalytic domain of the hammerhead ribozyme, and typically has the sequence ^{3′}CAAAGCAGGAGUGCCUGAGUAGUC^{5′} (SEQ ID NO:82). Variations on this sequence are known and are herein disclosed and incorporated by reference, for example, in the patents disclosed herein. A third part consists of sequence capable of hybridizing to the sequence immediately 3′ to the GUC cleavage site, and can be called a second hybridization arm. The hybiridization arms can be any length allowing binding to the substrate, such as, from 3-40 nucleotides long, 5-30 nucleotides long, 8-20, nucleotides long and 10-15 nucleotides long, as well as any length up to 50 nucleotides. As an example, hammerhead ribozymes can be designed by identifying a nucleic acid triplet GUC within the mRNA target sequence, and then identifying the appropriate hybridizing arms as discussed herein to the catalytic core as discussed herein. Examples of hammerhead ribozyme sequences are disclosed herein for IL-1α (SEQ ID NO:76), IL-1β (SEQ ID NO:77), COX-1 (SEQ ID NO:78), COX-2 (SEQ ID NO:79), cPGES (SEQ ID NO:81), and mPGES (SEQ ID NO:80), but it is understood that others are also disclosed as discussed herein. Furthermore, using assays as discussed herein, one can test a given ribozyme (or any functional nucleic acid, such as an siRNA or antisense) for its level of activity, both in vitro and in vivo.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)). Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules are found in the following non-limiting list of U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391, 806 811) (Napoli, C., et al. (1990) Plant Cell 2, 279 289) (Hannon, G. J. (2002) Nature, 418, 244 251). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200) (Bernstein, E., et al. (2001) Nature, 409, 363 366) (Hammond, S. M., et al. (2000) Nature, 404:293-296). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309 321). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-574). However, the effect of iRNA or siRNA or their use is not limited to anytype of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER siRNA Construction Kit. Disclosed herein are any siRNA designed as described above based on the sequences for the herein disclosed inflammatory mediators. Examples of siRNA include: COX-1 (SEQ ID NOs:47-52), COX-2 (SEQ ID NOs:53-58), cPGES (SEQ ID NOs:41-46), and mPGES (SEQ ID NO:59).

The production of siRNA from a vector is more commonly done through the transcription of a shRNA. Kits for the production of vectors comprising shRNA are available, such as for example Imgenex's GeneSuppressor Construction Kits and Invitrogen's BLOCK-iT inducible RNAi plasmid and lentivirus vectors. Disclosed herein are any shRNA designed as described above based on the sequences for the herein disclosed inflammatory mediators. Examples of shRNA primer sequences are disclosed for COX-1 (SEQ ID NOs:64-65), COX-2 (SEQ ID NOs:66-67), cPGES (SEQ ID NOs:60-61), and mPGES (SEQ ID NO:62-63).

b) Inhibition of Binding

Another activity of an inflammatory mediator that can be targeted is homophilic and heterophilic binding to other molecules, such as, for example, receptors. Thus, the inflammatory mediator inhibitor can be a ligand binding inhibitor. Methods for inhibiting the binding of a protein to its receptor can, for example, be based on the use of molecules that compete for the binding site of either the ligand or the receptor.

Thus, a ligand binding inhibitor can be, for example, a polypeptide that competes for the binding of a receptor without activating the receptor. Likewise, a ligand binding inhibitor can be a decoy receptor that competes for the binding of ligand. Such a decoy receptor can be a soluble receptor (e.g., lacking transmembrane domain) or it can be a mutant receptor expressed in a cell but lacking the ability to transduce a signal (e.g., lacking cytoplasmic tail). Antibodies specific for either a ligand or a receptor can also be used to inhibit binding. The ligand binding inhibitor can also be naturally produced by a subject. Alternatively, the inhibitory molecule can be designed based on targeted mutations of either the receptor or the ligand.

Thus, as an illustrative example, the ligand binding inhibitor can be IL-1 receptor antagonist (IL-1ra). The ligand binding inhibitor can also be a polypeptide comprising a fragment of IL-1ra, wherein the fragment is capable of binding IL-1R1. ligand binding inhibitor can further be IL-1R2, which is a soluble form of the receptor that can compete for IL-1 binding. The ligand binding inhibitor can further be a polypeptide comprising a fragment of IL-1R1. The IL-1R1 fragment can lack the cytoplasmic tail, which includes the Toll/interleukin-1(IL-1) receptor (TIR) domain (amino acids 384-528 of SEQ ID NO:8). The fragment of IL-1R1 can lack the amino acids corresponding to the transmembrane domain.

3. Inflammatory Mediators—Sequences

The disclosed inflammatory mediator can comprise a nucleic acid based on the sequence of IL-1 alpha. The nucleic acid sequence can be based on the sequence of human IL-1 alpha. An example of a nucleic acid encoding human IL-1 alpha is SEQ ID NO:1, Accession No. NM_{—}000575.

The disclosed inflammatory mediator can comprise a nucleic acid based on the sequence of IL-1 beta. The nucleic acid sequence can based on the sequence of human IL-1 beta. An example of a nucleic acid encoding human IL-1 beta is SEQ ID NO:2, Accession No. NM_{—}000576.

The disclosed inflammatory mediator can comprise a nucleic acid based on the sequence of IL-1ra. The nucleic acid sequence can based on the sequence of human IL-1ra. An example of a nucleic acid encoding human IL-1ra is SEQ ID NO:5, Accession No. NM_{—}173842.

The disclosed inflammatory mediator can comprise a nucleic acid based on the sequence of IL-1R1. The nucleic acid sequence can based on the sequence of human IL-1RA. An example of a nucleic acid encoding human IL-1R1 is SEQ ID NO:8, Accession No. NM_{—}000877.

The disclosed inflammatory mediator can comprise a nucleic acid based on the sequence of IL-1R2. The nucleic acid sequence can based on the sequence of human IL-1R2. An example of a nucleic acid encoding human IL-1R2 is SEQ ID NO:9, Accession No. NM_{—}173343.

The disclosed inflammatory mediator can comprise a nucleic acid based on the sequence of COX-1. The nucleic acid sequence can based on the sequence of human COX-1. An example of a nucleic acid encoding human COX-1 is SEQ ED NO:10, Accession No. M59979.

The disclosed inflammatory mediator can comprise a nucleic acid based on the sequence of COX-2. The nucleic acid sequence can based on the sequence of human COX-2. An example of a nucleic acid encoding human COX-2 (SEQ ID NO:11, Accession No. NM_{—}000963.

The disclosed inflammatory mediator can comprise a nucleic acid based on the sequence of mPGES. The nucleic acid sequence can based on the sequence of human mPGES. An example of a nucleic acid encoding human mPGES is SEQ ID NO:12, Accession No. NM_{—}004878.

The disclosed inflammatory mediator can comprise a nucleic acid based on the sequence of cPGES. The nucleic acid sequence can based on the sequence of human cPGES/p23. An example of a nucleic acid encoding human cPGES/p23 is SEQ ID NO:13, Accession No. L24804.

Disclosed herein is a functional nucleic acid wherein the nucleic acid can inhibit the expression of a mediator of inflammation. Also disclosed herein is a construct comprising a nucleic acid encoding the functional nucleic acid operably linked to an expression control sequence. The functional nucleic acid can comprise an siRNA. The siRNA can be derived from the nucleic acid sequence for COX-1 (SEQ ID NO:10). Thus, the siRNA can have the nucleic acid sequence SEQ ID NO:47, 48, 49, 50, 51, or 52. The siRNA can be derived from the nucleic acid sequence for COX-2 (SEQ ID NO:11). Thus, the siRNA can have the nucleic acid sequence SEQ ID NO:53, 54, 555, 56, 57, or 58. The siRNA can be derived from the nucleic acid sequence for mPGES (SEQ ID NO:12). Thus, the siRNA can have the nucleic acid sequence SEQ ID NO:59. The siRNA can be derived from the nucleic acid sequence for cPGES (SEQ ID NO:13). Thus, the siRNA can have the nucleic acid sequence SEQ ID NO:41, 42, 43, 44, 45, or 46.

Disclosed herein is a construct comprising a nucleic acid encoding a polypeptide operably linked to an expression control sequence, wherein the polypeptide can inhibit the binding of IL-1 to IL-1R1. The polypeptide can comprise IL-1ra. The polypeptide can have the amino acid sequence SEQ ID NO:38. The polypeptide can comprise a fragment of IL-1ra. The polypeptide can have at least 70%, 75%, 80%, 85%, 90%, 95% identity to the amino acid sequence SEQ ID NO:38. The nucleic acid can comprise the sequence SEQ ID NO:5. The nucleic acid encode a polypeptide that with at least 70%, 75%, 80%, 85%, 90%, 95% identity to the nucleic acid sequence SEQ ID NO:5. Also disclosed are nucleic acids that can hybridize under stringent conditions, or other conditions, as described herein, with the nucleic acid sequence SEQ ID NO:5.

The polypeptide can comprise a fragment of IL-1R1, wherein the fragment is capable of binding IL-1 and wherein the fragment has a reduced ability to activate a signal cascase. It is understood that one skilled in the art can readily determine the ability of a polypeptide to bind IL-1 or activate a signal cascase using standard biochemical and molecular genetics techniques and reagents. As an example, the fragment can be a truncation lacking the transmembrane domain. Wherein the transmembrane domain has not been identified, it is understood that one skilled in the art can estimate the approximate location of this domain based on the amino acid sequence using, for example, hydrophobicity plots. As another example, the fragment can lack part of the cytoplasmic tail, which includes the Toll/interleukin-1(IL-1) receptor (TIR) domain (amino acids 384-528 of SEQ ID NO:8). The polypeptide can have the amino acid sequence SEQ ID NO:39. The polypeptide can have at least 70%, 75%, 80%, 85%, 90%, 95% identity to the amino acid sequence SEQ ID NO:39. The nucleic acid can comprise the sequence SEQ ID NO:8. The nucleic acid encode a polypeptide that with at least 70%, 75%, 80%, 85%, 90%, 95% identity to the nucleic acid sequence SEQ ID NO:8. Also disclosed are nucleic acids that can hybridize under stringent conditions, or other conditions, as described herein, with the nucleic acid sequence SEQ ID NO:8.

The polypeptide can comprise IL-1R2. The polypeptide can have the amino acid sequence SEQ ID NO:40. The polypeptide can comprise a fragment of IL-1R2, wherein the fragment is capable of binding IL-1 and wherein the fragment has a reduced ability to activate a signal cascase. The polypeptide can have at least 70%, 75%, 80%, 85%, 90%, 95% identity to the amino acid sequence SEQ ID NO:40. The nucleic acid can comprise the sequence SEQ ID NO:9. The nucleic acid encode a polypeptide that with at least 70%, 75%, 80%, 85%, 90%, 95% identity to the nucleic acid sequence SEQ ID NO:9. Also disclosed are nucleic acids that can hybridize under stringent conditions, or other conditions, as described herein, with the nucleic acid sequence SEQ ID NO:9.

The herein disclosed polypeptides can further comprise a secretion signal. The secretion signal can be the IL-1ra secretion signal sequence, which is the same sequence as the secretion signal sequence of IL-1β. Thus, the secretion signal can comprise the polypeptide sequence SEQ ID NO:14. The secretion signal can be encoded by nucleic acid sequence SEQ ID NO:68.

The disclosed constructs can be integrated into a vector delivery system. Thus, disclosed are vectors comprising the nucleic acids provided herein. The expression control sequence is generally a promoter. The promoter can be any promoter, such as those discussed herein.

Targeted and global delivery of the constructs provided herein is also disclosed. Disclosed is a pseudotyped feline immunodeficiency virus (FIV) for global transgene delivery. Stable expression of the therapeutic gene aids prolonged restoration of the genetic anomaly enhancing treatment efficacy and contributing to long-term therapeutic outcomes. One of the backbone FIV systems disclosed herein is set forth in Poeschla E M, et al., Nature Medicine 4: 354-357. (1998). For example, disclosed herein is stable expression of the reporter gene lacZ for over 3 months in mice following perinatal systemic FIV(lacZ) administration.

A model system for the study of these constructs is the IL-1β exisionally activated transgenic (XAT) mouse (IL-1β^{XAT}) and variations thereof. Variations include the use of tissue specific promoters such as in for example the COLL1A1-IL-1β^{XAT} mouse. This mouse model is the subject of U.S. Patent Application No. 60/627,604, which is herein incorporated by reference in its entirety for teachings related to the disclosed mouse models. This mouse model allows for the induction of localized inflammation based on the delivery of a Cre recombinase expression vector such as FIV(Cre) to the target site. For example, the delivery of FIV(Cre) to the joints of the COLL1A1-IL-1β^{XAT} mouse can induce inflammation to model arthritis. This mouse model can thus be used to, for example, test or optimize the effects of the provided constructs on arthritis. Also disclosed herein is the ability of FIV vectors to deliver any of the herein provided nucleic acids or transgenes to the brain of a subject following injection of the vector into either the circulation or joints. Thus, the IL-1β^{XAT} and variations thereof can be used as a model of neuroinflammation following delivery of FIV(Cre) into the circulation or joints.

4. Compositions for Treating Pain

Disclosed are compositions for treating pain, including constructs and vectors for expressing one or more opioid receptors in a cell, such as a nerve cell, such as a peripheral nerve cell. As discussed herein, opioid receptors are typically expressed in the spinal or supraspinal nerve cells, and the periphery typically do not express these receptors. The disclosed compositions and methods are designed to express the opioid receptors in nerve cells which are damaged or transmitting because of trauma, but which do not have endogenous opioid receptors or insufficient numbers of endogenous receptors to react to the endogenous opioid like molecules, typically in the periphery of the nerve cell. Thus, the expression of the opioid receptors in the nerve cell near the point of pain, will typically increase the amount of opioid receptors in this area and thus, increase the responsiveness to endogenous opioid like molecules. By expression of the opioid receptors, the sensation of pain can be reduced, not by administration of opioid analgesics, but rather by more efficient use of endogenous opioid like compounds. It is understood, however, that opioids, opioid like molecules, and/or other pain alleviating molecules can be added in addition to the disclosed opioid receptors.

Disclosed are methods wherein administration occurs in the intra-articular region of the jaw. The results shown herein demonstrated that intra-articular injection of FIV(lacZ) resulted in successful gene transfer to articular TMJ surfaces as well as the joint meniscus. Thus, disclosed are methods, wherein the administration of the disclosed vectors, results in delivery to the articular TMJ surfaces and the joint meniscus.

Nociceptive innervation to the temporomandibular joint (TMJ) is primarily provided by the auriculotemporal nerve of the mandibular division of the trigeminal nerve (Sessle & Wu, 1991). AS and C nerve fibers, whose cell bodies are located in the posterolateral part of the trigeminal ganglion (Yoshino et al., 1998), project distally and terminate as non-encapsulated free nerve endings dispersed throughout the posterolateral part of the TMJ capsule (Bernick, 1962; Thilander, 1964; Frommer & Monroe, 1966; Klineberg, 1971), the posterior band of the meniscus and the posterior attachment (Dressen et al., 1990; Kido et al., 1991, 1993; Wink et al., 1992). Transfer of anti-nociceptive genes to sensory trigeminal neurons innervating the orofacial region can be achieved after injection of lentiviral vectors at the painful site, such as the TMJ, resulting in their uptake by free nerve endings and retrograde transport to the sensory cells' nuclei. Previous studies demonstrated axonal retrograde transport of horseradish peroxidase from the TMJ to the central nervous system (Romfh et al., 1979; Carpa, 1987) including the trigeminal ganglia (Yoshino et al., 1998).

Disclosed are constructs capable of expressing any of the opioid receptor gene products. Disclosed are constructs capable of expressing opioid receptors, such as the μ-opioid receptor gene product. The μ-opioid receptor construct allows for synthesis of μ-opioid receptor protein. The μ-opioid receptor construct typically comprises three parts: 1) a promoter, 2) the μ-opioid receptor coding sequence, and 3) polyA tail. The poly A tail can be from the bovine growth hormone or any polyA tail including synthetic poly A tails. The Bovine growth hormone poly A tail carries elements that not only increase expression, but also increase stability of any gene construct. These three parts can be integrated into any vector delivery system, which is capable of transducing terminally differentiated cells, such as nerve cells.

The promoter can be any promoter, such as those discussed herein. It is understood as discussed herein that there are functional variants of opioid receptors, such as the μ-opioid receptor protein which can be made. In certain embodiments the promoter is going to be a cell specific promoter, such as a nerve cell specific promoter, such as the neuron specific enolase promoter. Other promoters are disclosed herein.

The promoter can be any promoter, such as those discussed herein. It is understood as discussed herein that there are functional variants of opioid receptors, such as the μ-opioid receptor protein which can be made. In certain embodiments the promoter is going to be a cell specific promoter, such as a nerve cell specific promoter, such as the neuron specific enolase promoter.

μ-opioid receptor cDNA can be obtained from the American Tissue Culture Collection. (American Tissue Culture Collection, Manassas, Va. 20110-2209; μ-opioid receptor ATCC#. Raynor K, et al., Characterization of the cloned human mu opioid receptor. J Pharmacol Exp Ther. 1995; 272:423-8.)

Also disclosed are constructs encoding for the human or mouse μ-opioid receptor, as well as the β-galactosidase reporter gene (lacZ).

Disclosed are nucleic acids comprising sequence encoding μ-opioid receptor. Also disclosed are nucleic acids, wherein the nucleic acid further comprises a promoter sequence, wherein the μ-opioid receptor has at least 80% identity to the sequence set forth in SEQ ID NO:93 or 95,wherein the receptor has at least 85% identity to the sequence set forth in SEQ ID NO:92 or 94, wherein the μ-opioid receptor has at least 90% identity to the sequence set forth in SEQ ID NO:92 or 94, wherein the μ-opioid receptor has at least 95% identity to the sequence set forth in SEQ ID NO:92 or 94, and/or wherein the μ-opioid receptor has the sequence set forth in SEQ ID NO: 92 or 94.

Also disclosed are vectors comprising the disclosed nucleic acids. Also disclosed are cells comprising the disclosed nucleic acids and vectors. Any cell can be targeted with the disclosed constructs. However, nerve cells, for example, are terminally differentiated. This means that they are no longer dividing. The state of a mature non-dividing nerve cell can define terminally differentiated cells. In terms of differentiated\stable transduction, nerve cells thus represent attractive targets because once DNA is integrated, there is a very low probability that it will not remain in the cell.

Also disclosed are non-human mammals comprising the disclosed nucleic acids, vectors, and cells disclosed herein. Also disclosed are methods of providing μ-opioid receptor in a cell comprising transfecting the cell with the nucleic acids. Also disclosed are method of delivering the disclosed compositions, wherein the transfection occurs in vitro or in vivo. Disclosed are methods of making a transgenic organism comprising administering the disclosed nucleic acids, vectors and/or cells.

Disclosed are methods of making a transgenic organism comprising transfecting a lentiviral vector to the organism at during a perinatal stage of the organism's development. Strategies of producing genetically engineered pluripotent, such as embryonic, stem cells, can be performed with the disclosed compositions to produce engineered cells and organisms as discussed herein. Furthermore by cloning strategies can be used to produce desried organisms, which carry one or more of the disclosed compositions.

Also disclosed are methods of treating a subject having pain comprising administering any of the disclosed compounds and compositions. Delivery of the disclosed constructs to terminally differentiated cells is also disclosed. Disclosed is a pseudotyped feline immunodeficiency virus (FIV) for μ-opioid receptor delivery to terminally differentiated cells. Stable expression of the therapeutic gene aids prolonged expression, enhancing treatment efficacy and contributing to long-term therapeutic outcomes. The backbone FIV system has been shown to effectively incorporate, due to its lentiviral properties, the transgene of interest into the host's genome, allowing for stable gene expression (Poeschla et al., 1998). Disclosed herein is stable expression of the reporter gene lacZ in N2a cells, following perinatal systemic FIV(lacZ) administration.

In certain embodiments the constructs become an integrated product with the genome of the host. For example, lentiviruses, such as HIV and LIV, have the characteristic of transfecting the therapeutic gene into the host chromosome, thus forming an integrated product. In certain embodiments, the requirement is that the vectors allow for expression in the periphery of the cell, such as the nerve cell, and/or at or near the point of pain. The contrast to integrated products is episomal products which can also be produced using, for example, HSV and AV vectors. Thus, transient expression can be beneficial. The optimal time of expression is correlated with the amount of product produced and amount that is needed. For example, in certain embodiments, expression for at least 3, 4, 5, 6, 7, 8, 9, 10, 15, 20, 30, 45, 60, 90, 120, 150, or 180 days is desirable.

5. Opioid Receptors

There are typically considered three classes of opioid receptor μ, δ and κ. Genes encoding for these receptors have been cloned (Evans et al (1992) Science 258 1952; Kieffer et al (1992) Proc. Natl. Acad. Sci. USA 89 12048; Chen et al (1993) Mol. Pharmacol. 44 8; and Minami et al (1993) FEBS Lett. 329 291 all of which are herein incorporated by reference for material related to opioid receptors and there sequence). In addition, an orphan receptor was identified which has a high degree of homology to the known opioid receptors and based on structural grounds it is considered a receptor called ORL1 (opioid receptor-like) (Mollereau et al (1994) FEBS Lett. 341 33, herein incorporated by reference for material related to opioid receptors and there sequence). Since the cloned receptors function as opioid receptors, by for example interacting with pertussis toxin-sensitive G-proteins, all of the cloned opioid receptors possess the same general structure which includes an extracellular N-terminal region, seven transmembrane domains and intracellular C-terminal tail structure. Evidence obtained from pharmacokinetic and activity data indicate there are subtypes of each receptor and other types, such as less well-characterized opioid receptors, such as ε, λ, l, ζ, which are known. One way of characterizing the different receptor subtypes for μ-, δ- and κ-receptors is through different post-translational modifications of the gene product (glycosylation, palmitoylation, phosphorylation, etc). Also receptor dimerization to form homomeric and heteromeric complexes or from interaction of the gene product with associated proteins such as RAMPs can effect function, and thus represent another way to characterize the receptors. Different opioids have different affinity for the different opioid receptors. For example, μ-morphine, δ-leukenkephalin metenkephalin, κ-dynorphin, β-endorphin, have different affinities for the various opioid receptors.

a) μ-Receptor Subtypes

The MOR-1 gene, encoding for one form of the p-receptor, shows approximately 50-70% homology to the genes encoding for the δ-(DOR-1), κ-(KOR-1) and orphan (ORL1) receptors. Two different splice variants of the MOR-1 gene have been cloned, and they differ by 8 amino acids in the C-terminal tail which are either present or not. The splice variants exhibit differences in their rate of onset and recovery from agonist-induced internalization but their pharmacology does not appear to differ in ligand binding assays. A MOR-1 knockout mouse has been made and the mouse does not respond to morphine, by failing to alleviate pain, and by failing to exhibit positive reinforcing properties or an ability to induce physical dependence in the absence of the MOR-1 gene. This indicates that at least in this species, morphine's analgesia is not mediated through δ- or κ-receptors. (Matthes et al (1996) Nature 383 818).

The μ receptor is divided into the μ1 and μ2 groups. The division occurs because of binding and pharmaco activity studies which indicate, for example, that naloxazone and naloxonazine abolish the binding of radioligands to the μ1-site, and in vivo studies showed that naloxazone selectively blocked morphine-induced antinociception but did not block morphine-induced respiratory depression or the induction of morphine dependence, indicating different types of μ-receptor (Ling et al (1984) Science 226 462 and Ling et al (1985) J. Pharmacol. Exp. Ther. 232 149). Subsequent work in other laboratories has failed to confirm this classification.

Peptide sequences of the human and mouse μ receptor are set forth in SEQ ID Nos 92 and 94 respectively.

There is also data consistent with a third form of μ receptor where analogues of morphine with substitutions at the 6 position (e.g. morphine-6b-glucuronide, heroin and 6-acetyl morphine) are agonists, but with which morphine itself does not interact (Rossi et al (1996) Neuroscience Letters 216 1, herein incorporated by reference for material at least related to opioid receptors and their function and structure). Antinociception tests on mice show that morphine does not exhibit cross tolerance with morphine-6b-glucuronide, heroin or 6-acetyl morphine. Furthermore, in mice of the CXBX strain morphine is a poor antinociceptive agent whereas morphine-6b-glucuronide, heroin and 6-acetyl morphine are all potently antinociceptive. Heroin and morphine-6-glucuronide, but not morphine, still produce antinociception in MOR-1 knockout mice in which the disruption in the MOR-1 gene was engineered in exon-1 (Schuller et al (1999) Nature Neuroscience 2 151). Furthermore, all three agonists were ineffective as antinociceptive agents, in MOR-1 knockout mice in which exon-2, not exon-1, had been disrupted. This indicates that the antinociceptive actions of heroin and morphine-6-glucuronide in the exon-1 MOR-1 mutant mice are mediated through a receptor produced from an alternative transcript of the MOR-1 gene differing from the MOR-1 gene product, the μ-opioid receptor, in the exon-1 region.

b) δ-Receptor Subtypes

Only one δ-receptor gene has been cloned (DOR-1), but overlapping subdivisions of δ-receptor have been proposed (δ1/δ2 and δcx/δncx) on the basis of in vivo and in vitro pharmacological experiments.

The δ receptor subclasses arise from pharmacological studies. Results from in vivo rodent studies are shown in Table 1.

TABLE 1

| | | | Non- |
|---|---|---|---|
| | Agonist | Competitive antagonist | competitive antagonist |
| δ1 | DPDPE/DADLE | BNTX (7- | DALCE ([D-Ala2, D- |
| | | benzylidenenaltrexone) | Leu5]enkephalyl-Cys) |
| δ2 | Deltorphin II/ | Naltriben | 5′-NTII (naltrindole 5′- |
| | DSLET | | isothiocyanate |

The pharmacological properties of the cloned DOR-1 receptor are somewhere between those predicted for either the δ1 or δ2 subtypes. Mouse and human recombinant receptors both bind DPDPE and deltorphin II, which can displacer of [3H]-diprenorphine. This is different than either a δ1 or δ2 classification (Law et al (1994) J. Pharmacol. Exp. Ther. 271 1686). [3H]-diprenorphine binding to the mouse recombinant receptor, however, is more highly displaced by naltriben than BNTX, consistent with it being δ2 like.

Opioid receptors have also been indicated to be in complex μ-receptors and κ-receptors. For example, one type of δ receptor subtypes complexes, δcx, and another appears not to complex, δncx (Rothman et al (1993) In: Handbook Exp. Pharmacol. Ed. A. Herz 104/1 p217).

c) κ-Receptor

The cloned κ-Receptor has the sequence set forth in SEQ ID NO: 96, which represents an example of a κ-receptor.

d) The Orphan Opioid Receptor

The orphan receptor has been identified in three species: rat, mouse and man, all having a greater than 90% identity with each other. This receptor is typically referred to as ORL-1 for orphan receptor like 1. The endogenous peptide agonist for ORL1 is known as nociceptin or orphanin FQ. While the ORL1 receptor has structural homology to orphan receptors it does not have pharmacological homology. Non-selective ligands that exhibit high affinity for all μ-, κ- and δ-receptors, have very low affinity for the ORL1 receptor. Comparison of the deduced amino-acid sequences of the four receptors highlights structural differences consistent with the lack of coligand binding. The trans-membrane regions are conserved near their top in the μ-, κ- and δ-receptors, but are altered in ORL1. Site-directed mutants of ORL1 towards the traditional receptors (rat) are able to bind the traditional receptor's ligands. For example, benzomorphan bremazocine binds ORL1 by changing Ala213 in TM5 to the conserved Lys of μ, κ and δ, or by changing the Val-Gln-Va1276-278 sequence of TM6 to the conserved Ile-His-Ile motif (Meng et al (1996) J. Biol. Chem. 271 32016). There are also a number of splice variants of the ORL1 receptor, XOR (Wang et al (1994) FEBS Lett. 348 75) with a long form (XOR1L) containing an additional 28 amino acids in the third extracellular loop and in the homologous receptor from mouse, KOR-3, five splice variants have been reported to date (Pan et al (1998) FEBS Lett. 435 65).

e) Endogenous Ligands

In mammals the endogenous opioid peptides are mainly derived from four precursors: pro-opiomelanocortin, pro-enkephalin, pro-dynorphin and pro-nociceptin/orphanin FQ. Nociceptin/orphanin FQ is processed from pro-nociceptin/orphanin FQ and is the endogenous ligand for the ORL1-receptor; it has little affinity for the μ- and δ- and κ-receptors. Table 3 sets forth endogenous ligands for the opioid receptors. These peptides bind μ, δ- and κ-receptors with different affinity, and have negligible affinity for ORL1-receptors, but none binds exclusively to one opioid receptor type. β-endorphin is equiactive at μ- and δ-receptors with much lower affinity for κ-receptors; the post-translational product, N-acetyl-β-endorphin, has very low affinity for any of the opioid receptors. [Met]- and [Leu]enkephalin have high affinities for δ-receptors, ten-fold lower affinities for μ-receptors and negligible affinity for κ-receptors. Other products of processing of pro-enkephalin, which are N-terminal extensions of [Met] enkephalin, have a decreased preference for the δ-receptor with some products, e.g. metorphamide displaying highest affinity for the μ-receptor. The opioid fragments of pro-dynorphin, particularly dynorphin A and dynorphin B, have high affinity for κ-receptors but also have significant affinity for μ- and δ-receptors.

Endomorphin-1 and endomorphin-2 are putative products of an as yet unidentified precursor, that have been proposed to be the endogenous ligands for the μ-receptor where they are highly selective. The endomorphins are amidated tetrapeptides and are structurally unrelated to the other endogenous opioid peptides (Table 3). Although the study of the cellular localization of these peptides is at an early stage, endomorphin-2 is found in discrete regions of rat brain, some of which are known to contain high concentrations of μ-receptors. Endomorphin-2 is also present in primary sensory neurones and the dorsal horn of the spinal cord where it could function to modulate nociceptive input.

In comparison to the mainly non-selective mammalian opioid peptides (the exceptions being the endomorphins), amphibian skin contains two families of D-amino acid-containing peptides that are selective for μ- or δ-receptors. Dermorphin is a μ-selective heptapeptide Tyr-D-Ala-Phe-Gly-Tyr-Pro-Ser-NH2 without significant affinity at d- and k-receptors. In contrast, the deltorphins—deltorphin (dermenkephalin; Tyr-D-Met-Phe-His-Leu-Met-Asp-NH2), [D-Ala2]-deltorphin I and [D-Ala2]-deltorphin II (Tyr-D-Ala-Phe-Xaa-Val-Val-Gly-NH2, where Xaa is Asp or Glu respectively)—are highly selective for δ-opioid receptors. Table 3 shows a variety of endogenous opioid receptor molecules.

TABLE 2
Endogenous opioid receptor molecules

| | | Amino |
|---|---|---|
| Precursor | Endogenous peptide | acid sequence |
| Pro- | β-Endorphin | YGGFMTSEKSQTP |
| opiomelanocortin | | LVTLFKNAIIKNA |
| | | YKKGE |
| | | SEQ ID NO: 129 |
| Pro-enkephalin | [Met]enkephalin | YGGFM |
| | | SEQ ID NO: 130 |
| | [Leu]enkephalin | YGGFL |
| | | SEQ ID NO: 131 |
| | | YGGFMRF |
| | | SEQ ID NO: 132 |
| | | YGGFMRGL |
| | | SEQ ID NO: 133 |
| | Metorphamide | YGGFMRRV-NH₂ |
| | | SEQ ID NO: 134 |
| Pro-dynorphin | Dynorphin A | YGGFLRRIRPKLK |
| | | WDNQ |
| | | SEQ ID NO: 135 |
| | Dynorphin A(1-8) | YGGFLRRI |
| | | SEQ ID NO: 136 |
| | Dynorphin B | YGGFLRRQFKVVT |
| | | SEQ ID NO: 137 |
| | α-neoendorphin | YGGFLRKYPK |
| | | SEQ ID NO: 138 |
| | β-neoendorphin | YGGFLRKYP |
| | | SEQ ID NO: 139 |
| Pro-nociceptin/ | Nociceptin | FGGFTGARKSARK |
| OFQ | | LANQ |
| | | SEQ ID NO: 140 |
| Pro-endomorphin* | Endomorphin-1 | YPWF-NH₂ |
| | | SEQ ID NO: 141 |
| | Endomorphin-2 | YPFF-NH₂ |
| | | SEQ ID NO: 142 |

Opioid receptor activation produces a wide array of cellular responses (Table 2). For example, there are Direct G-protein bg or a subunit-mediated effects, such as activation of an inwardly rectifying potassium channel, inhibition of voltage operated calcium channels (N, P, Q and R type), inhibition of adenylyl cyclase, Responses of unknown intermediate mechanism, activation of PLA2, activation of PLC b (possibly direct G protein bg subunit activation), activation of MAPKinase, activation of large conductance calcium-activated potassium channels, activation of L type voltage operated calcium channels, inhibition of T type voltage operated calcium channels, and direct inhibition of transmitter exocytosis. There are also responses in other effector pathways, such as activation of voltage-sensitive potassium channels (activation of PLA2), inhibition of M channels (activation of PLA2), inhibition of the hyperpolarisation-activated cation channel (Ih) (reduction in cAMP levels following inhibition of adenylyl cyclase), elevation of intracellular free calcium levels (activation of PLCb, activation of L type voltage operated calcium conductance), potentiation of NMDA currents (activation of protein kinase C), inhibition of transmitter release (inhibition of adenylyl cyclase, activation of potassium channels and inhibition of voltage operated calcium channels), decreases in neuronal excitability (activation of potassium channels), increases in neuronal firing rate (inhibition of inhibitory transmitter release—disinhibition), and changes in gene expression (long-term changes in adenylyl cyclase activity, elevation of intracellular calcium levels, activation of cAMP response element binding protein (CREB)).

6. Nucleic Acids

There are a variety of molecules disclosed herein that are nucleic acid based, including for example the nucleic acids that encode, for example, IL-1ra as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, IL-1ra as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

7. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

8. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

9. Delivery of the Compositions to Cells

The herein disclosed nucleic acids can be delivered to cells or cells in a subject. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as, for example, the IL-1ra, COX-1 siRNA, COX-2 siRNA, cPGES siRNA, or mPGES siRNA constructs into the cell without degradation and include a promoter yielding expression of the disclosed sequences in the cells into which it is delivered. In some embodiments the vectors for the IL-1ra, COX-1 siRNA, COX-2 siRNA, cPGES siRNA, or mPGES siRNA constructs are derived from a virus, retrovirus, or lentivirus. Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone, and lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene, such as, the disclosed IL-1ra, COX-1 siRNA, COX-2 siRNA, cPGES siRNA, or mPGES siRNA constructs or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector, which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(4) Lentiviral Vectors

The vectors can be lentiviral vectors, including but not limited to, SIV vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also vectors can be any viral family that share the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV.

(a) Feline Immunodeficiency Viral Vectors

One type of vector that the disclosed constructs can be delivered in is the VSV-G pseudotyped Feline Immunodeficiency Virus system developed by Poeschla et al. (1998). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns et al., 1993) contributes to the formation of the viral envelope in trans. The third vector confers packaging instructions in trans (Poeschla et al., 1998). FIV production is accomplished in vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

(5) Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system”J. Virol 72(11):8463-71 (1998)

Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

(6) Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

(7) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, in addition to the disclosed nucleic acids or vectors, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

10. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

Expression vectors used in eukaryotic host cells (yeast, fungi, insect, plant, animal, human or nucleated cells) can also contain sequences necessary for the termination of transcription which could affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

In certain embodiments the promoters are constitutive promoters. This can be any promoter that causes transcription regulation in the absence of the addition of other factors. Examples of this type of promoter are the CMV promoter and the beta actin promoter, as well as others discussed herein. In certain embodiments the promoter can consist of fusions of one or more different types of promoters. For example, the regulatory regions of the CMV promoter and the beta actin promoter are well known and understood, examples, of which are disclosed herein. Parts of these promoters can be fused together to, for example, produce a CMV-beta actin fusion promoter, such as the one shown in SEQ ID NO:18. It is understood that this type of promoter has a CMV component and a beta actin component. These components can function independently as promoters, and thus, are themselves considered beta actin promoters and CMV promoters. A promoter can be any portion of a known promoter that causes promoter activity. It is well understood that many promoters, including the CMV and Beta Actin promoters have functional domains which are understood and that these can be used as a beta actin promoter or CMV promoter. Furthermore, these domains can be determined. For example, SEQ ID NO:s 15-33 display a number of CMV promoters, beta actin promoters, and fusion promoters. These promoters can be compared, and for example, functional regions delineated, as described herein. Furthermore, each of these sequences can function independently or together in any combination to provide a promoter region for the disclosed nucleic acids.

The promoters can also be non-constitutive promoters, such as cell specific promoters. These are promoters that are turned on at specific time in development or stage or a particular type of cell, such as a cardiac cell, or neural cell, or a bone cell. Some examples of cell specific promoters are, the neural enolase specific promoter (NSE), the procollagen promoters COL1A1 (SEQ ID NO:35) and COL2A1 (SEQ ID NO:36), the CD11b promoter (PBMC-microglia/macrophage/monocyte specific) (SEQ ID NO:69), and the glial specific glial fibrillary acetic protein (GFAP) promoter (SEQ ID NO:34).

It is understood that the recombinant systems can be expressed in a tissue-specific manner. It is understood that tissue specific expression can occur due to the presence of a tissue-specific promoter. Typically, proteins under control of a tissue-specific promoter are transcribed when the promoter becomes active by virtue of being present in the tissue for which it is specific. Therefore, all cells can encode for a particular gene without global expression. As such, labeled proteins can be shown to be present in certain tissues without expression in other nearby tissues that could complicate results or expression of proteins in tissues where expression is detrimental to the host. Disclosed are methods wherein the cre recombinase is under the control of the EIIA promoter, a promoter specific for breast tissue, such as the WAP promoter, a promoter specific for ovarian tissue, such as the ACTB promoter, or a promoter specific for bone tissue, such as osteocalcin. Any tissues specific promoter can be used. Promoters specific for prostate, testis, and neural are also disclosed. Examples of some tissue-specific promoters include but are not limited to MUC1, EIIA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promote, Pdha-2. For example, use of the AFP promoter creates specificity for the liver. Another example, HOXA-1 is a neuronal tissue specific promoter, and as such, proteins expressed under the control of HOXA-1 are only expressed in neuronal tissue. Sequences for these and other tissue-specific promoters are known in the art and can be found, for example, in Genbank, at www.pubmed.gov.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

c) Post Transcriptional Regulatory Elements

The disclosed vectors can also contain post-transcriptional regulatory elements. Post-transcriptional regulatory elements can enhance mRNA stability or enhance translation of the transcribed mRNA. An exemplary post-transcriptional regulatory sequence is the WPRE sequence isolated from the woodchuck hepatitis virus. [Zufferey R, et al., J Virol; 73:2886-92 (1999)]. Post-transcriptional regulatory elements can be positioned both 3′ and 5′ to the exogenous gene, but it is preferred that they are positioned 3′ to the exogenous gene.

d) Transduction Efficiency Elements

Transduction efficiency elements are sequences that enhance the packaging and transduction of the vector. These elements typically contain polypurine sequences. An example of a transduction efficiency element is the ppt-cts sequence that contains the central polypurine tract (ppt) and central terminal site (cts) from the HIV-1 pSG3 molecular clone (bp 4327 to 4483 of HIV-1 pSG3 clone).

e) 3′ Untranslated Regions

Expression vectors used in eukaryotic host cells (yeast, fungi, insect, plant, animal, human or nucleated cells) may also contain sequences necessary for the termination of transcription which could affect mRNA expression. These 3′ untranslated regions are transcribed as polyadenylated segments in the untranslated portion of the mRNA encoding the exogenous gene. The 3′ untranslated regions also include transcription termination sites. The transcription unit also can contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In an embodiment of the transcription unit, the polyadenylation region is derived from the SV40 early polyadenylation signal and consists of about 400 bases. Transcribed units can contain other standard sequences alone or in combination with the above sequences improve expression from, or stability of, the construct.

11. Peptides

a) Protein Variants

Disclosed herein are constructs comprising nucleic acids that encode polypeptides. As discussed herein, there can be numerous variants of each of these polypeptides, such as IL-1ra, that are herein contemplated. In addition, to the known functional proteins that are disclosed, such as IL-1ra, there are also derivatives of these proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 3 and 4 and are referred to as conservative substitutions.

TABLE 3
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 4
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:5 sets forth a particular sequence of IL-1ra and SEQ ID NO:9 sets forth a particular sequence of a IL-1R2 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂ —CH═CH— (cis and trans), —COCH₂—, —CH(OH)CH₂—, and —CHH₂S)—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—) each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

12. Pharmaceutical Carriers/Delivery of Pharmaceutical Products

The compositions disclosed herein can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like could be necessary or desirable.

Compositions for oral administration include powders or granules, suspensions or solutions in water or non-aqueous media, capsules, sachets, or tablets. Thickeners, flavorings, diluents, emulsifiers, dispersing aids or binders could be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions can be determined empirically, and making such determinations is within the skill in the art. The dosage ranges for the administration of the compositions are those large enough to produce the desired effect in which the symptoms of disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as a vector, for treating, inhibiting, or preventing inflammation, the efficacy of the therapeutic vector can be assessed in various ways well known to the skilled practitioner. For instance, one of ordinary skill in the art will understand that a composition, such as a vector, disclosed herein is efficacious in treating or inhibiting inflammation in a subject by observing that the composition reduces inflammation.

13. Animals

Provided herein are transgenic animals comprising germline transmission of any of the vectors or nucleic acids provided herein. In one aspect, the transgenic animal provided herein is an excision activated transgenic (XAT) animal. The disclosed transgenic animals can have temporally and spatially regulated transgene expression (Brooks, A I, et al. 1991. Nature Biotech 15:57-62; Brooks, A I, et al. 1999. Neuroreport 10:337-344; Brooks, A I., et al. 2000. Proc Natl Acad Sci USA 97:13378-13383) of an inflammation element. It is understood that where the transgenic animal comprises a nucleic acid comprising a recombination site, as disclosed herein, delivery of a recombinase, such as Cre recombinase to cells within the provided transgenic animal will result in the expression of the inflammatory modulator, e.g., IL-1β, IL-1ra, COX-2, within those cells.

By a “transgene” is meant a nucleic acid sequence that is inserted by artifice into a cell and becomes a part of the genome of that cell and its progeny. Such a transgene an be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This can include genes which are normally be present in the nontransgenic genome but which one desires to have altered in expression, or which one desires to introduce in an altered or variant form. A transgene can include one or more transcriptional regulatory sequences and any other nucleic acid, such as introns, that can be necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

The disclosed transgenic animals can be any non-human animal, preferably a non-human mammal (e.g. mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule can be integrated within a chromosome, or it can be extrachromosomally replicating DNA. For techniques related to the production of transgenic animals, see, inter alia, Hogan et al (1986) Manipulating the Mouse Embryo—A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1)and F (2)). The present invention further provides transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

The provided compositions can be evaluated using a mouse model of arthritis. As prolonged expression of IL-1β in the joint can lead to the development of arthrosis similar to that seen in arthritis patients, disclosed is a mouse model of arthritis based on prolonged, low level intra-articular transgenic expression of IL-1β. The role of IL-1β, TNFα and other inflammatory mediators, such as prostanoids, are well recognized in the pathogenesis of arthritis. The two most commonly forms of arthritis are osteoarthritis (OA), which affects about 80%-90% of all adults over the age of 65, and rheumatoid arthritis (RA), which affects approximately 1% of the general U.S. population. Although distinct differences exist between OA and RA, both appear to develop secondary to a pro-inflammatory cascade. Previous animal models have proven valuable in studying arthritis and testing novel therapies, including the model of methylated bovine serum albumin/IL-1β, intra-articular administration of IL-1β, constitutive intra-articular expression of IL-1β following ex vivo transfer of genetically engineered synoviocytes, as well as the TNFα transgenic mouse model. The aforementioned IL-1β models are based on the direct administration of a deleterious agent, whereas the TNFα transgenic mouse is based on the prolonged expression of TNFα in vivo and has thus far yielded valuable insight on the role of TNFα in the development of arthritis. However, as with the majority of transgenic mice, TNFα transgenesis is susceptible to uncontrolled and uncharacterized developmental compensatory changes.

The provided mouse model is based on a method (somatic mosaic analysis) utilizing a germline transmitted recombinational substrate containing a dormant transcription unit and somatic gene transfer of a viral vector that expresses Cre recombinase that “activates” the gene of interest. IL-1β excisionally activated transgenic (IL-1β^{XAT}) mice, and variations thereof, have been generated using this method. The provided mouse model is the subject of U.S. Patent Application No. 60/627,604, which is herein incorporated by reference in its entirety. This mouse model allows for the induction of localized inflammation based on the delivery of a Cre recombinase expression vector such as FIV(Cre) to the target site. Variations include the use of cell or tissue specific promoters such as in for example the COL1A1-IL-1β^{XAT} mouse. For example, the delivery of FIV(Cre) to, for example, the joints of the COLL1A1-IL-1β^{XAT} mouse can induce inflammation to model arthritis. This mouse model can thus be used to, for example, test or optimize the effects of the provided constructs on arthritis. As another example, delivery of FIV(Cre) to the circulation or joint of the COLL1A1-IL-1β^{XAT} mouse can induce inflammation in the brain to model, for example, Alzheimer's disease.

IL1β^{XAT} regulation is controlled in a temporal (time) and spatial (location) fashion by the Cre/loxP molecular genetic method utilizing (1) a germline transmitted recombinational substrate (e.g. COLL1-IL1β^{XAT}) containing a dormant transcription unit and (2) somatic gene transfer of a viral vector that expresses Cre recombinase which “activates” the gene of interest. Thus, these mice can be used herein to induce IL-1β constitutive expression in the joints (e.g., knee) of mice. As an example, localized transgene activation, i.e., IL-1β, can be accomplished in IL-1β^{XAT} mice by the intracapsular injection of FIV(Cre), a lentivirus capable of transducing soft and hard tissues of joints, to the area of interest, and subsequent recombinational excision of the [symbol] STOP [symbol] cassette leading to gene transcription. Recombination-mediated gene “activation” permanently alters the genetic constitution of infected cells thus allowing chronic IL-1β synthesis. The COLL1A1 promoter can further be used to target gene expression to chondrocytes, osteocytes and fibroblasts, making this transgenic mouse available for the study of arthritis in any joint of interest. This promoter has been shown to target gene expression in bone and cartilage and was cloned in the IL-1β^{XAT} gene in place of the CMV promoter:

(COLL1A1-IL1β^{XAT}) COLL1A1=> [symbol] STOP [symbol] ssIL1β-IRES-lacZ

COLL2 is another suitable promoter. This transgene has been constructed and tested in a murine NIH 3T3 stable cell line following expression of Cre recombinase by the transient transfection of the pRc/CMV-CreWT expression vector or after infection by the lentiviral vector FIV(Cre).

The somatic gene transfer of the recombinase, such as Cre can be performed using any type of vector system producing the recombinase. However, in certain embodiments, the vector system is a self inactivating vector system, wherein the promoter, for example, of the recombinase is flanked by recombination sites so that upon production of the recombinase, the recombinase will down regulate its own production. The delivery vectors for the recombinase can be CRE mediated.

For example, activation of the dormant COLL1-IL1β^{XAT} can be mediated by the transfer of Cre recombinase to the area of interest (e.g. knee) via a self-inactivating Cre feline immunodeficiency virus FIV(Cre). The effects of this FIV vector system have been previously examined using the reporter gene lacZ (β-galactosidase) in mice that received intra-articular injections of a viral solution [Kyrkanides S, et al. (2004). J Dental Res 83: 65-70], wherein transduction of soft (articular disc) and hard (cartilage) TMJ tissues was demonstrated. The FIV(Cre)vector has been constructed by cloning a loxP-flanked (“floxed”) nlsCre cassette in the place of the lacZ gene; the nuclear localization signal (nls) was fused to the cre open reading frame by PCR and subsequently cloned into the TOPO 2.1 vector (Invitrogen) per manufacturer's instructions employing a custom-made floxed cloning cassette. The reason for developing a self-inactivating cre gene is based on a recent paper [Pfeifer A and Brandon E P, Kootstra Neeltje, Gage F H, Verma I M (2001). Proc Natl Acad Sci U.S.A. 98: 11450-5], whereby the authors reported cytotoxicity due to prolonged expression of Cre recombinase mediated by infection using a lentiviral vector. In the provided construct, upon production of adequate levels of Cre recombinase to produce excisional activation of COLL1-IL1β^{XAT} following successful transduction of target cells with FIV(Cre), Cre is anticipated to de-activate the cre gene by loxP-directed self excisional recombination.

14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:5, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a promoter element and a nucleic acid element disclosed herein. The nucleic acid element can, for example, encode a ligand binding inhibitor. Thus, disclosed are nucleic acid molecules produced by the process comprising linking in an operative way a promoter element and an IL-1ra element. Also disclosed is a nucleic acid molecule produced by the process comprising linking in an operative way a promoter element and an IL-1R2 element. Also disclosed is a nucleic acid molecules produced by the process comprising linking in an operative way a promoter element and an IL-1R1 fragment element. Also disclosed is a nucleic acid molecules produced by the process comprising linking in an operative way a promoter element and an IL-1 fragment element.

Also disclosed are nucleic acid molecules produced by the process comprising linking in an operative way a promoter element and a nucleic acid element wherein the nucleic acid encodes a gene expression inhibitor disclosed herein. As an example, disclosed are nucleic acid molecules produced by the process comprising linking in an operative way a promoter element and a COX-1 siRNA element. Also disclosed are nucleic acid molecules produced by the process comprising linking in an operative way a promoter element and a COX-2 siRNA element. Also disclosed are nucleic acid molecules produced by the process comprising linking in an operative way a promoter element and a mPGES siRNA element. Also disclosed are nucleic acid molecules produced by the process comprising linking in an operative way a promoter element and cPGES siRNA element.

Further disclosed are cells produced by the process of transforming the cell with any of the disclosed nucleic acids. Disclosed are cells produced by the process of transforming the cell with any of the non-naturally occurring disclosed nucleic acids.

Disclosed are any of the peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Also disclosed are mammals wherein mammal is a murine, ungulate, or non-human primate.

Also disclose are animals produced by the process of adding to the animal any of the cells disclosed herein.

### E. Methods of Using the Compositions

1. Methods of Using the Compositions as Research Tools

The disclosed compositions can be used in a variety of ways as research tools. For example, the disclosed compositions, such as SEQ ID NOs:5 can be used to study the interactions between IL-1 and IL-1R1, by for example acting as inhibitors of binding.

2. Therapeutic Uses

Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of a disclosed composition, such as the disclosed constructs, for treating, inhibiting, or preventing inflammation, the efficacy of the therapeutic construct can be assessed in various ways well known to the skilled practitioner. For instance, one of ordinary skill in the art will understand that a composition, such as the disclosed constructs, disclosed herein is efficacious in treating inflammation or inhibiting or reducing the effects of inflammation in a subject by observing that the composition reduces the onset of the conditions associated with these diseases. Furthermore, the amount of protein or transcript produced from the constructs can be analyzed using any diagnostic method. For example, it can be measured using polymerase chain reaction assays to detect the presence of construct nucleic acid or antibody assays to detect the presence of protein produced from the construct in a sample (e.g., but not limited to, blood or other cells, such as neural cells) from a subject or patient.

### F. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Acceleration of Chondrocyte Maturation During Perinatal Development Underlies Abnormal Skeletogenesis in Lysosomal Storage Disorders

a) Materials and Methods

HexB−/− knockout mice were originally developed using 129S4 ES cells into C57BL/6 embryos and subsequently maintained on a 129S4 background (Sango, K., et al. 1996). The original strains are commercially available by the Jackson Laboratory (Bar Harbor, Me.; strain designations: B6;129S4-Hexatm1Rlp/J and B6;129S4-Hexbtm1Rlp/J, respectively). In total, 31 mice were employed in this study: HexB^{-/-} (N=16), hexB^{+/-} (N=6) and wild type (N=9) mice were produced by routine animal mating strategies and genotyping as previously described (Kyrkanides, S., et al. 2005). In brief, HexB^{+/-} knockout breeder pairs on pure 129S4 background were mated to produce homozygous HexB^{-/-} knockout mice at a 0.25 expectancy ratio. Genotyping was performed by PCR of DNA extracts from tail biopsies employing the following primer sets: 5′ATT TTA AAA TTC AGG CCT CGA3′ (SEQ ID NO:126), 5′CAT AGC GTT GGC TAC CCG TGA3′ (SEQ ID NO:127) and 5′CAT TCT GCA GCG GTG CAC GGC3′ (SEQ ID NO:128). The latter were allowed to grow to maturity (60 days old) and were than employed as breeders to deliver HexB^{-/-} pups at a 1.00 expectancy ratio for the subsequent experiments.

Development of viral vectors and animal injections: Construction of the bicistronic transgene HEXB-IRES-HEXA encoding both subunits of the human β-hexosaminidase (pHEX) was previously described (Kyrkanides, S., et al. 2003). In brief, the human HexB cDNA was isolated from the pHexB43 plasmid (ATCC, Manassas Va.) by Xho I digestion and insertion into the Xho I site of pIRES expression vector (Clonetech). The HexA cDNA was isolated from pBHA-5 (ATCC) following Xho I digestion and subsequently inserted into the Xba I site of pIRES vector by blunt ligation. The CMV promoter drives transgene expression, and the translation of the second open reading frame, HexA, is facilitated by an internal ribosomal entry sequence (IRES): CMV-HEXB-IRES-HEXA-pA.

The defective FIV vector CTRZlb (Poeschla, E. M., et al. 199), which served as the backbone for the development of FIV(HEX), and both pseudotyping (Burns, J. C., et al. 1993) and packaging plasmids were kindly provided by Dr. David Looney (University of California at San Diego). In brief, the Nhe I-Not I segment containing the CMV-HEXB-IRES-HEXA construct was cloned in the place of lacZ in the CTRZLb vector (SstII-Not I) by blunt-cohesive ligation to generate the FIV(HEX) transfer vector (Kyrkanides, S., et al. 2005). FIV vectors were packaged in 293H cells as previously described (Kyrkanides, S., et al. 2005; Kyrkanides, S., et al. 2003). Briefly, T75 flasks were seeded with 293H cells which were grown to subconfluency in DMEM plus 10% FBS (Gemini, Woodland Calif.). The cells were then cotransfected with the transfer vector, pFIV(HEX), the packaging and the VSV-G pseudotyping vectors using the Lipofectamine 2000 reagent (Invitrogen) per manufacturer's instructions. Twenty-four hours after transfection, the supernatant medium was discarded and replaced by fresh medium. Sixty hours after transfection, the virus-rich supernatant was collected, filtered through 0.45 mm Surfil®-MF filter (Corning Seperations Division, Acton Mass.), and subsequently concentrated by overnight centrifugation at 7,000 g using a Sorvall RC5B high speed centrifuge and a SLA-3000 rotor. Subsequently, the supernatant was decanted and the viral pellet resuspended overnight in 1 mL of normal buffered saline containing 40 mg/mL lactose at 4° C. The viral solution was then aliquoted and frozen (−80° C.) until further use. Titers were calculated at 10⁸ infectious particles/mL for FIV(HEX) by X-HEX histochemistry in CrfK cells (American Tissue Culture Collection; Manassas, Va.) cultured in 24 well tissue culture plates (Kyrkanides, S., et al. 2003). The effectiveness of FIV(HEX) to transduce murine cells was previously tested in vitro in primary murine fibroblasts and primary human fibroblasts derived from a patient suffering from Tay-Sachs disease (Coriell Institute for Medical Research; cat. No. GM11853; Camden N.J.) as well as in Sandhoff mice in vivo (Kyrkanides, S., et al. 2005). HexB^{-/-} knockout neonates were injected intraperitoneally at postnatal day P4 with 10⁷ infectious FIV(HEX) particles in 100 μl normal saline.

Cephalometric radiography: Cephalometric analysis provided quantitative information related to the growth of the craniofacial skeleton. In brief, the animals were anesthetized by ketamine (40 mg/Kg) intraperitoneal injection, immobilized on a customized cephalostat with their cranial mid-sagittal plane positioned parallel to the cephalometric film cassette, and radiographs were obtained utilizing a long-cone X-ray machine at preset distances as previously described (Fujita, T., et al. 2004). The cranial and nasomaxillary measurements in each animal were normalized in reference to the length of the mandibular corpus and expressed as ratios. Using this method, craniofacial morphology was examined in mice at 8 and 16 weeks of age. Statistical analysis was undertaken using analysis of variance methods with α=0.05 and Tukey post-hoc analysis. All landmark identification and measurements were performed by one investigator (PK) and the intra-examiner reliability was calculated by correlation coefficient on 10 radiographs as r>0.9 prior to the commencement of the study.

Histological studies: Histological analysis of long bone growth plates and cranial base synchondroses was performed in samples obtained from 16 week old Hex^{-/-} mice. In brief, mice were deeply anesthetized by intraperitoneal injection of ketamine (40 mg/Kg) and pentobarbital (100 mg/Kg). Under surgical plane of anesthesia, the mice were transcardially perfused by 100 mL of 4% paraformaldehyde in phosphate buffered saline. Subsequently, the cranial bases were dissected, de-fleshed and decalcified by immersion in an EDTA solution for 7 days in 4° C. under constant agitation. The tissues were then processed on a RHS-1 microwave tissue processor, after which the samples were embedded in paraffin. Tissues were cut on a microtome at 3 μm thick sections and the presence of cartilage in the synchondroses was detected by Alcian blue hematoxylin—orange G histochemistry.

Immunohistochemical analysis was performed for a number of antigens. In general, the tissue slides were first deparaffinized in xylene, rehydrated through graded alcohols and quenched in 3% H₂O₂ for 20 min. Antigen retrieval was performed in a pressure cooker using a 10 mM citrate buffer pH 6.0. For collagen II (Col-2), the tissue was also digested with pepsin (0.2%). Subsequently, the tissue was blocked using appropriate primary serum solution followed by overnight incubation in primary antibody solution at 4° C. The following morning, the sections were rinsed with PBS and incubated in an appropriate biotinylated secondary antibody solution for 30 min, followed by PBS wash and incubated in horseradish peroxidase-conjugated streptavidin. AEC was employed as chromagen. Sections were counterstained with hematoxylin, followed by PBS wash, alcohol dehydration, xylene clearing and cover-slipped permanent mounting media. Specifically, a goat anti-Col-2 was purchased from Lab Vision Corp. (Fremont Calif.) and was used at 1:40 dilution; a goat anti-parathyroid related peptide (PTHrP) antibody (dilution 1:40) was purchased from Santa Cruz Biotechnology Inc. (Santa Cruz Calif.). The rabbit anti-(murine) COX-2 and EP2 antibodies were purchased from Cayman (Ann Arbor Mich.), and the rabbit anti-active p38 antibody from Promega (Madison Wis.). Appropriate biotin conjugated secondary antibodies were purchased from Jackson Immunoresearch (West Grove Pa.).

In vitro studies: The C₂C₁₂ cell line, an in vitro model of chondrocyte differentiation and maturation, was obtained from ATCC and cultured in DMEM plus 10% normal bovine serum for 4 days as previously described (Katagiri, T., et al. 1994). In addition, cells were treated with BMP-2 (300 ng/mL) or alternatively with BMP-2 plus PGE₂ (10⁻⁸M) or recombinant IL-1β (10 ng/mL) or butaprost (10⁻⁸M) in the culture medium. At the end of the experiment, cells were fixed with 10% formalin. Alkaline phosphatase expression was evaluated using the BCIP/NBT histochemistry method (Vector Labs, Burlingame Calif.). Positively-stained cells were counted in 10 random 20× fields using an inverted Olympus CK41 microscope.

b) Results

Lysosomal storage disorders, including Sandhoff disease, often manifest skeletal malformations of the long bones as well as the craniofacial skeleton, with the latter often being the first and foremost feature noticed in affected patients (Gorlin, R. J., et al. 1991). In order to quantitatively evaluate the degree of skeletal impairment, lateral cephalometric analysis of the craniofacial skeleton was performed, a method routinely employed in the detailed evaluation of skeletal defects in human patients.

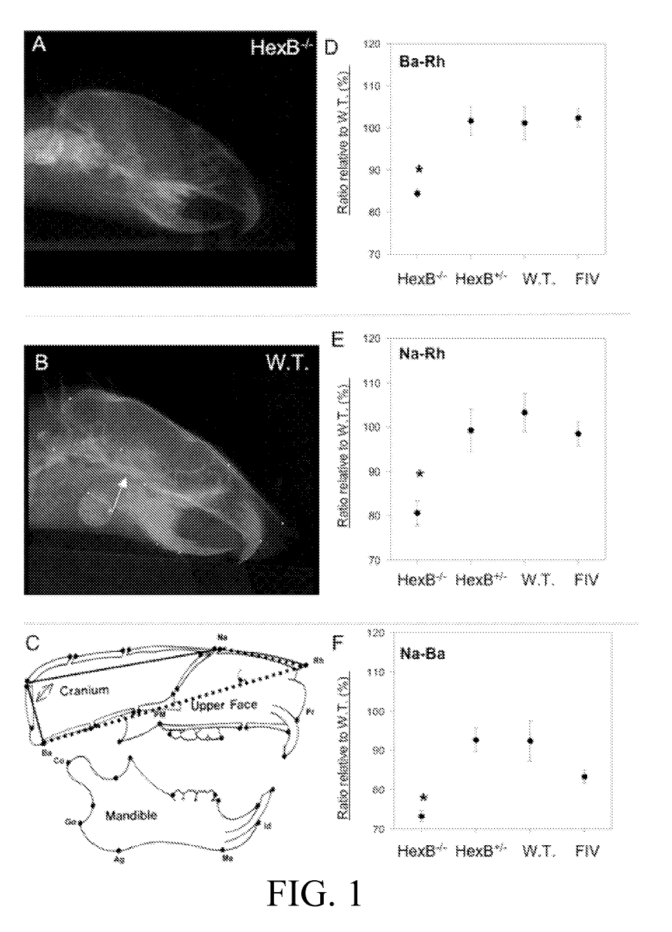

Craniofacial skeletal impairment in Sandhoff mice: To determine the craniofacial skeletal structures affected by β-hexosaminidase deficiency in a quantitative manner, cephalometric analyses employing angular and linear measurements on lateral cephalometric radiographs were obtained from HexB^{-/-}, HexB^{+/-} and wild type littermates. The data revealed that HexB^{-/-} knockout mice were characterized by shorter nasomaxillary depth (Na—Rh), shorter craniofacial depth (Ba—Rh) and shorter cranial base depth (Ba—Na) compared to HexB^{+/-} and wild type mice (FIG. 1). Interestingly, there were no differences in mandibular size in the animals examined (P>0.1). Since no differences between the 8 and 16 week time points were identified, only the 8 week results are shown herein. Overall growth was evaluated by measuring gross weight on a weekly basis and no differences were noted between the animal groups (P>0.1). In conclusion, the aforementioned analyses indicated that the craniofacial skeletal impairment is localized in the animals' cranial base, a bony structure comprised of two important bone growth sites, the spheno-occipital and pre-sphenoid synchondroses (specialized growth plates). Consequently, the cranial base synchondroses were evaluated in β-hexosaminidase and wild type mice by histological and immunohistochemical methods.

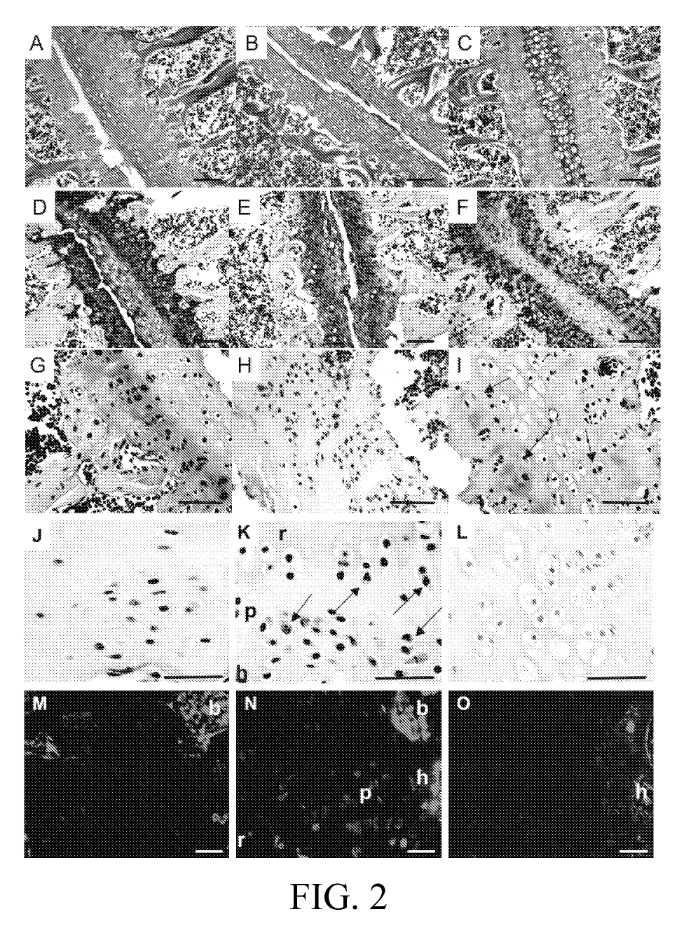

Chondrocyte phenotypic switch in the growth plates of HexB^{-/-} mice: To examine the underlying etiology of the skeletal defects developing secondary to β-hexosaminidase deficiency at the cellular level, the spheno-occipital synchondrosis (SOS) of the cranial base were evaluate as well as the femur and tibia growth plates by histological and immunohistochemical techniques in HexB^{-/-} and wild type mice. Qualitatively, HexB^{-/-} SOS manifested a decrease in extracellular cartilaginous content and aberrant ectopic bone formation along with chondrocyte hyperplasia (FIG. 2). Furthermore, a loss of normal SOS cyto-architecture was evident in the mutant mouse, characterized by the absence of chondrocyte column formation in the proliferative zone and the complete lack of a resting zone (FIG. 2). Changes in the expression of markers associated with skeletogenesis were also observed, including a decrease in PTHrP expression (an inhibitor of chondrocyte maturation) and induction of TRAP and VEGF (indicators of hypertrophic-terminally mature chondrocytes) along with a significant increase of COX-2 expression in chondrocytes (FIG. 2).

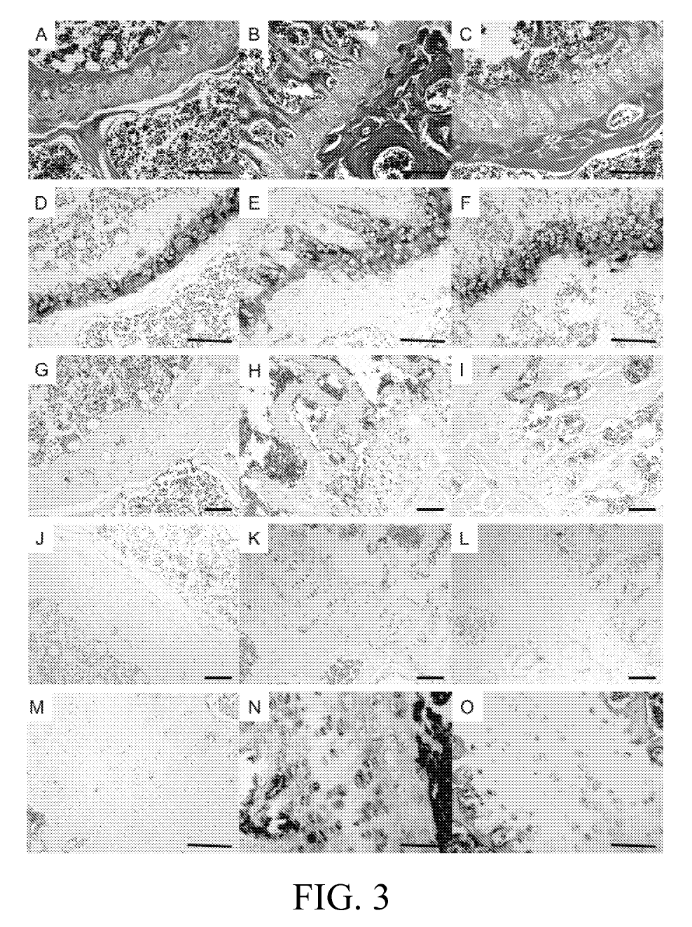

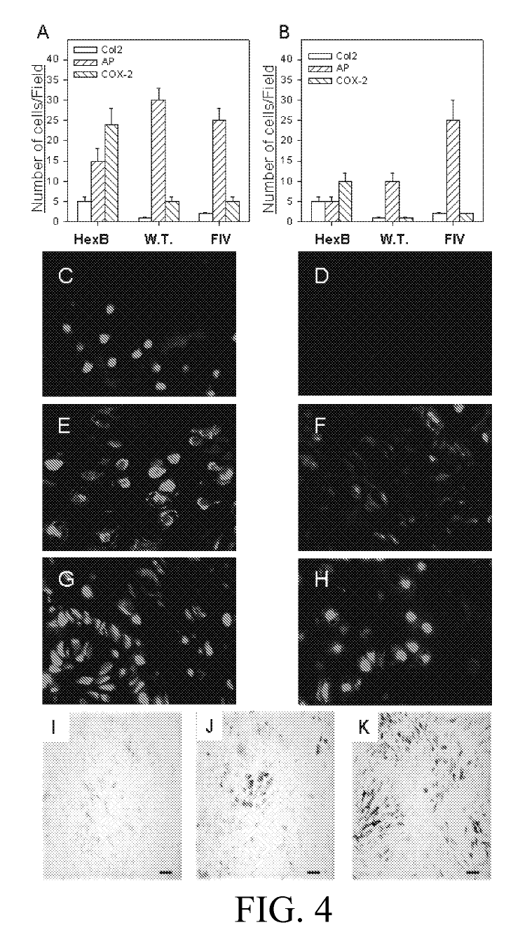

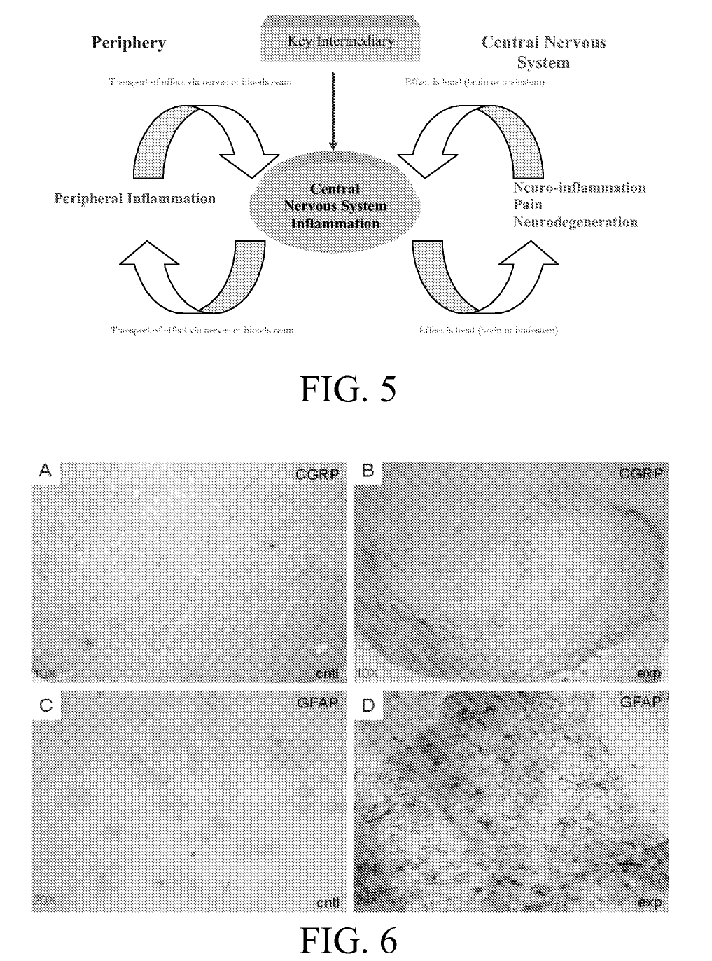

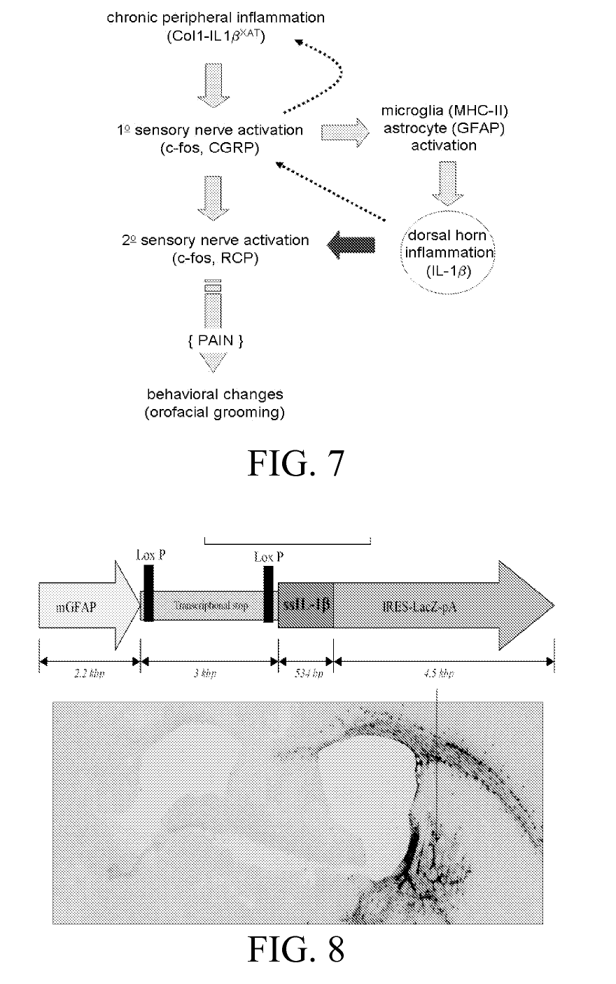

To determine if the aforementioned changes are limited to the cranial base synchondroses or whether are also present in growth plates of other endochondrally-derived bones, histological and immunohistochemical analysis of long bone growth plates (FIG. 3) was pursued. Qualitatively, HexB^{-/-} femur and tibia presented with loss of normal growth plate cyto-architecture, chondrocyte hyperplasia and increased woven bone formation. Also, a striking increase of TRAP expression was observed in the HexB^{-/-} growth plates compared to wild type littermates. Quantitative analysis of the aforementioned immunohistochemical analyses revealed a significant increase in the number of Col-2, TRAP and COX-2 expressing chondrocytes in the long bone growth plates and the cranial base synchondroses (FIG. 4). Therefore, the aforementioned data indicate a cellular phenotypic switch of proliferative/pre-hypertrophic chondrocytes in wild type mice towards a hypertrophic-terminally mature chondrocyte in the Sandhoff mice.

Neonatal β-hexosaminidase restitution rescues HexB^{-/-} skeletal development: To determine the developmental window during which β-hexosaminidase deficiency affects chondrocyte maturation, we rescued Sandhoff mice from β-hexosaminidase deficiency at a neonatal stage of development. To this end, we employed a previously developed method (Kyrkanides, S., et al. 2003), the systemic transfer of a therapeutic gene by the recombinant feline immunodeficiency virus FIV(Hex). Eight and 16 weeks following FIV(Hex) administration to HexB^{-/-} neonates, a significant attenuation of craniofacial growth and development was observed at the clinical level as assessed by cephalometric radiography (FIG. 1). Furthermore, 16 weeks following viral transduction, histological analysis of the cranial base synchondroses revealed normalization of the cyto-architecture in the cranial base synchondroses (FIG. 2) and long bone growth plates and (FIG. 3). Immunohistochemical analysis of bone markers revealed that neonatal FIV(Hex) treatment in Sandhoff neonates restored the expression of PTHrP, attenuated the expression of Col-2 as well as TRAP (FIGS. 2, 3 & 4). Interestingly, the presence of the therapeutic gene was not observed in growth plate chondrocyte.

The COX-PG pathway is implicated in the HexB^{-/-} craniofacial phenotype: To begin exploring the possible mechanisms that mediate the effects of β-hexosaminidase deficiency on chondrocyte maturation, the expression of COX-2 was evaluated in the growth plates of Sandhoff and wild type mice, a known stimulator of chondrocyte differentiation and maturation. COX-2 expression was elevated in the cranial base synchondroses and long bone growth plates in HexB^{-/-} knockout mice (FIGS. 2 & 3). Neonatal FIV(Hex) therapy resulted in amelioration of this COX-2 induction in HexB^{-/-} mice, indicating a possible link between β-hexosaminidase deficiency, COX-2 induction and chondrocyte maturation. In fact, the stress-activated p38 MAK, a known stimulator of COX-2, was also induced in growth plate chondrocytes (FIG. 4). COX-2 is rate limiting in the production of prostanoids and prostaglandin PGE₂ in particular, the effects of which are mediated by EP receptors, including EP2 that was found present in HexB^{-/-} and wild type growth plate chondrocytes.

The effects of the COX-PG pathway in chondrocyte maturation were evaluated in vitro by employing the C₂C₁₂ cell line (FIG. 4). Administration of PGE₂ to differentiating C₂C₁₂ cells under the stimulus of BMP-2 resulted in acceleration of their conversion to an osteoblastic phenotype (number of cells converted in a defined period of time), indicating that activation of the COX-PG pathway in HexB^{-/-} chondrocytes may in fact induce the acceleration of chondrocyte maturation.

### 2. Example 2

### Glial Cells and IL1β in the Processing of Pain

a) Methods

FIV vectors. Three types of FIV viral vectors can be used: (A) FIV(Cre) and (B) FIV(gfp) and FIV(IL1ra), which encode for Cre recombinase and the reporter gene green fluorescent protein (gfp) and IL1ra receptor antagonist, respectively. FIV vectors can be prepared, packaged and concentrated as previously described (Kyrkanides et al., 2004, 2005). A total of 10⁶ infectious particles in 10 μL of viral solution can be injected intraarticularly in the TMJ of mice under surgical plane of anesthesia. Similarly, 2 μl of viral solution will be injected into the cisterna magna as described below.

TMJ Histopathology. At each time point, a subset of mice can be anesthetized by CO₂ inhalation and decapitated immediately. Their heads can be harvested, de-fleshed and immersed in 10% formalin solution for fixation. Subsequently, the specimens can be decalcified in EDTA solution, processed and paraffin embedded. Histology TMJ sections can then be cut and collected onto glass slides, deparaffinized and analyzed by histochemical stains and immunocytochemistry. Serial parasagittal sections collected every 100 μm covering the entire TMJ condyle can be evaluated under 40× magnification. This technique produces 15 sections per TMJ. (1) First, the TMJ sections can be stained by H&E and Alcian blueorange G stain. Degenerative changes in the articular cartilage can be evaluated and graded in examined under light microscope, and scored into five categories according to Wilhelmi and Faust (1976) and Helminen et al. (1993): grade 0, no apparent changes; grade 1, superficial fibrillation of articular cartilage; grade 2, defects limited to uncalcified cartilage; grade 3, defects extending into calcified cartilage; and grade 4, exposure of subchondral bone at the articular surface. Each TMJ can be graded according to the highest score observed within the serial sections. (2) Activation and expression of the IL1β^{XAT} transgene can be accomplished by immunocytochemistry (ICC) for human mature IL-1β as well as bacterial β-galactosidase (lacZ) employing commercially available antibodies. (3) The expression of a number of arthritis-related genes can be assessed by immunocytochemistry, such as murine IL-1β, IL-6, COX-2, MMP-9, col2 and ADAMST5. (4) Possible infiltration of inflammatory cells can be detected using antibodies raised against the following antigens: monocytes/macrophages by Mac-1/MHC-II; lymphocytes by CD-3 as previously described (Kyrkanides et al. 2003, 2004). Also neutrophils can be detected by a rat anti-murine neutrophil antibody (Serotec, Raleigh, N.C.). (5) Apoptosis and proliferation can be evaluated by TUNEL and PCNA immunocytochemistry, respectively. The identity of the cells can be confirmed by double immunocytochemistry. In all instances, quantification of the number of cells can be described both in terms of number of positive cells per field, as well as staining profile (Kyrkanides et al. 2002, 2003).

Brain stem and ganglia histology. After the mice have been euthanized, the brain stem and trigeminal ganglia can be harvested and fixed by immersion into 10% formalin solution (Kyrkanides et al. 2002, 2004). In brief, brain stems can be sectioned horizontally at 18 μm and the sections will be collected on glass slides in a serial manner. Sections covering the entire region of interest (−5 mm-to-+10 mm relative to obex for the brain stem and the entire trigeminal ganglia) can be included from each animal in the studies. Neuroinflammation: The development of inflammation in the brain stem and ganglia can be evaluated by immunocytochemistry on histology sections using established methods (employing antibodies raised against glial fibrillary acidic protein (GFAP) and major histo-compatibility complex II (MHC-II). In addition, the expression of inflammatory cytokines, such as IL-1β, IL1-RI, IL1ra, TNFα and IL-6, as well as inducible members of the cyclooxygenase pathway (COX-2, mPGES-1) can also be evaluated.

Trigeminal excitation: Excitation of the sensory component of the trigeminal cranial nerve can be assessed at the level of the trigeminal ganglia and the brain stem trigeminal nuclear complex (including the main sensory nucleus, descending track and nucleus of trigeminal cranial nerve) by immunocytochemistry. For this purpose, the expression of pain-related excitatory neurokines can be evaluateed, including substance P (SP) and calcitonin gene related peptide (CGRP), as well as p38 MAP kinase and c-fos.

Quantification of mRNA Abundance by Real-Time RT-PCR. Quantification of mRNA levels is accomplished using an ICYCLER (Bio-Rad) and real time qRT-PCR with TAQMAN probes constructed with FAM as the fluorescent marker and Blackhole I quencher (Biosearch Technologies, Novato Calif.). Prior to PCR of the cDNA samples, PCR conditions are optimized for each mRNA to be analyzed. Standard curve reactions are performed by varying annealing temperatures, primer concentrations, and Taqman probe concentration. Serial dilution of the starting cDNA template demonstrate linear amplification over at least 5 orders of magnitude.

PCR reactions are performed in a volume of 25 μl and contained iQ Supermix (Bio-Rad, Hercules Calif.; 0.625 U Taq, 0.8 mM dNTP, 3 mM Mg2+, 0.2-0.6 μM concentrations of each primer, 10-100 nM probe and 1 μl of cDNA sample. To ensure consistency, a master mix is first prepared containing all reagents except the cDNA sample. Primers are designed using the Primer Express (Applied Biosystems) and Oligo 6.83 programs (Molecular Biology Insights, Inc., Cascade, Colo.). In general, PCR reaction conditions are the following: denaturation at 95° C. for 3 min, followed by 40 cycles of amplification by denaturing at 95° C. for 30 s, annealing at 60° C. for 30 s and extension at 72° C. for 60 s. For each real time PCR, a standard curve is performed to insure direct linear correlation between product yield (expressed as the number of cycles to reach threshold) and the amount of starting template. The correlation is always greater than r=0.925. PCR reaction efficiency (e) is determined for each reaction. To correct for variations in starting RNA values, the level of ribosomal 18S RNA or GAPDH RNA is determined for all samples and used to normalize all subsequent RNA determinations. Normalized threshold cycle (Ct) values are then transformed, using the function-expression=(1+e)Ct, in order to determine the relative differences in transcript expression. Data are compared by ANOVA and Tukey's post hoc tests, and by linear regression to determine correlations using the JMP statistics program (SAS Institute). A probability of P<0.05 will be considered statistically significant.

TABLE 5
Real-Time RT-PCR Primers
COX-2 (Optimal annealing temp: 60°)

| | |
|---|---|
| 820-UP 20 mer | tga ccc cca agg ctc aaa ta |
| | SEQ ID NO: 143 |
| 821-LP 21 mer | ccc agg tcc tcg ctt atg atc |
| | SEQ ID NO: 144 |
| 822-PR 27 mer | ctttgcccagcacttcacccatcagtt |
| | SEQ ID NO: 145 |

IL-1β, murine (Optimal annealing temp: 60°)

| | |
|---|---|
| 838-UP 20 mer | tcg ctc agg gtc aca aga aa |
| | SEQ ID NO: 146 |
| 839-LP 22 mer | atcagaggcaaggaggaaacac |
| | SEQ ID NO: 147 |
| 840-PR 29 mer | catggcacattctgttcaaagagagcctg |
| | SEQ ID NO: 148 |

TNFα (Optimal annealing temp: 55°)

| | |
|---|---|
| 892-UP 19 mer | gac aag gct gcc ccg act a |
| | SEQ ID NO: 149 |
| 893-LP 26 mer | ttt ctcctggtatgagatagcaaatc |
| | SEQ ID NO: 150 |

G3PDH (Optimal annealing temp: 60°)

| | |
|---|---|
| 823-UP 18 mer | ccc aat gtg tcc gtc gtg |
| | SEQ ID NO: 151 |
| 824-LP 20 mer | cct gct tca cca cct tct tg |
| | SEQ ID NO: 152 |
| 825-PR 29 mer | tgtcatatacttggcaggtttctccagg |
| | SEQ ID NO: 153 |

IL-6 (Optimal annealing temp: 60°)

| | |
|---|---|
| 853-UP 23 mer | ccagaaaccgctatgaagttcct |
| | SEQ ID NO: 154 |
| 854-LP 20 mer | caccagcatcagtcccaaga |
| | SEQ ID NO: 155 |
| 855-PR 27 mer | tctgcaagagacttccatccagttgcc |
| | SEQ ID NO: 156 |

### 3. Example 3

### Chronic TMJ Arthritis Induce Glial Activation and Neuroinflammation in the Brain Stem

Glial cell activation can be examined in the brain stem and the trigeminal ganglia following the development of chronic TMJ arthritis in the Col1-IL1β^{XAT} transgenic mouse model. Since glial activation has been previously implicated in the development of brain inflammation, the development of brain stem neuroinflammation can be investigated in this TMJ arthritis mouse model. The advantage of this strategy, in contrast to previous models (i.e. careegenan, Freuds adjuvant, formalin injections) is the employment of the Col1-IL1β^{XAT} transgenic mouse model that allows for the induction of chronic peripheral (TMJ) inflammation and the study of central changes over a period of weeks-months.

a) Methods

FIV(Cre) intra-articular bilateral injection into the right and left TMJ of adult Col1-IL1β^{XAT} transgenic mice induces transgene activation and subsequently the development of TMJ arthritis and pain as early as eight weeks following viral transduction. Following the induction of TMJ inflammatory pain in young adult (2 month old) Col1-IL1β^{XAT} transgenic mice, the development of neuroinflammation and the excitation of the trigeminal sensory system can be temporally and spatially characterized at the brain stem and trigeminal ganglia, and the development of pain can be behaviorally evaluated in vivo. For example, 4 groups of mice can be used in this experiment: (a) Col1-IL1β^{XAT} transgenic mice injected intra-articularly with FIV(Cre) in both the left and left sides to develop TMJ arthritis and inflammatory pain; (b) Col1-IL1β^{XAT} transgenic mice injected intra-articularly with FIV(gfp), a viral vector capable of transducing mammalian cells with the reported gene green fluorescent protein, controls for the effects of viral intraarticular transduction; (c) Col1-IL1β^{XAT} transgenic mice injected with sterile saline controls for the effects of the injection procedure; (d) wild type littermates injected by sterile saline controls for possible aging effects.

The mice can be examined at the following 3 time points: 2 weeks, 2 months and 6 months after TMJ arthritis induction. These time points were chosen based on data, whereby behavioral changes suggestive of pain in experimental mice were first seen as early as 2 weeks after transgene activation in the TMJ of Col1-IL1β^{XAT} transgenic mice. Moreover, a 2 month and a 6 month time point are included to temporally characterize the potential development of brain neuroinflammation, which in turn can elucidate the events preceding the possible development of chronic pain. To this end, disclosed is astrocyte activation at the main sensory nucleus and subnucleus caudalis of the trigeminal cranial nerve in the brain stem of Col1-IL1β^{XAT} transgenic mice 8 weeks after the induction of TMJ arthritis.

b) Experimental Outcomes

Neuroinflammation: The development of inflammation in the brain stem following peripheral inflammatory pain can be evaluated in experimental and control mice at the histology and molecular levels. Specifically, glial cell activation can be examined first by immunocytochemistry on brain stem histology sections using established methods employing antibodies raised against glial fibrillary acidic protein (GFAP), a marker of astrocyte activation, and major histo-compatibility complex II (MHC-II), a marker of microglia activation. In addition, the expression of inflammatory cytokines, such as IL-1β, IL1-RI, IL1ra, TNFα and IL-6, as well as inducible members of the cyclooxygenase pathway (COX-2, mPGES-1) can also be evaluated by immunohistochemistry. At the molecular level, an array of inflammatory genes, including IL-1β, TNFα, IL-6, iNOS, IL1-RI, IL1ra, COX-2 and mPGES-1 can be analyzed in the mRNA level by quantitative real time polymerase chain reaction (qRT-PCR).

Trigeminal excitation: Excitation of the sensory component of the trigeminal cranial nerve can be assessed at the level of the trigeminal ganglia and the brain stem trigeminal nuclear complex (including the main sensory nucleus, descending track and nucleus of trigeminal cranial nerve) by immunocytochemistry. For this purpose, the expression of pain-related excitatory neurokines, including substance P (SP) and calcitonin gene related peptide (CGRP), as well as p38 MAP kinase and c-fos, can be evaluated

TMJ inflammation: The development of inflammation in the TMJ can be assessed at the histology and molecular levels. To this end, the expression of inflammatory mediators associated with arthritis, such as IL-1β, TNFα, IL-6, COX-2, mPGES-1 and MMP-9, can be evaluated by immunohistochemistry on TMJ histology sections as well as by qRT-PCR in TMJ tissue harvested from experimental and control mice.

Pain Behavior: Orofacial pain can be evaluated at the behavioral level by assessing orofacial grooming and resistance to mandibular opening.

### 4. Example 4

### Effect of Brain Stem Neuroinflammation on the Processing of Orofacial Pain

Glial activation and neuroinflammation can exacerbates nociception through the central expression of inflammatory mediators, such as IL-1β, and subsequent neuronal excitation. Orofacial pain can be evaluated following the central induction of acute, short-term and long-term neuroinflammation in the brain stem of adult mice. To this end, three mouse models of neuroinflammation can be employed.

Acute model: This model is based on a single intracisternal injection of IL-1β (10 ng in 2 μL of aqueous solution) in adult wild type mice at the level of the brain stem via direct administration into the cisterna magna, the anatomical cavity located posterior to brain stem and inferior to the cerebellum. The central effects of IL-1β via this method can endure for a period of 36-60 hours.

Short-term model: This model is based on the cannulation of the cisterna magna with a pediatric catheter and the sustained release of IL-1β (or IL-1β neutralizing antibody in Col1-IL1β^{XAT} transgenic mice) over a period of 2 weeks powered by an osmotic mini-pump implanted subdermally in the back of the mice.

Long-term model: This model is based on the somatic mosaic analysis in the brain stem of adult GFAP-IL1β^{XAT} transgenic mice. The GFAP-IL1β^{XAT} transgenic mouse is an in vivo model of chronic neuroinflammation based on the sustained expression of IL-1β by astrocytes in the central nervous system following transgene activation by Cre recombinase using an FIV(Cre) virus. To this end, a single intracisternal injection of FIV(Cre) into the cisterna magna of adult GFAP-IL1β^{XAT} transgenic mice will activate the permanent release of IL-1β and subsequently cause the development of chronic neuroinflammation at the brain stem.

These 3 models of neuroinflammation offer a distinct advantage as it allows investigation of the effects of IL1β-based neuroinflammation on the central processing of pain over three complimentary time periods ranging form 2-3 days-to-6 months.

a) Effect of Acute Brain Stem Neuroinflammation on Neuronal Excitation and Hyperalgesia or Spontaneous Nociception

IL-1β can be administered intrathecally via a single injection into the cisterna magna of adult male (2 month old) wild type mice (C57/B16) under surgical plane of anesthesia. Sixty hours later, the mice can be evaluated for centrally-induced changes in behavior (spontaneous nociception) as assessed by orofacial grooming and resistance to mouth opening and make comparisons to the behavioral baseline measurements (prior to IL-1β injection). An additional group of mice can receive an equal volume of sterile saline via the same route of administration and serve as controls. Moreover, the development of hyperalgesia can be evaluated. A subset mice an be further challenged by intra-articular injection of formalin (0.625% in saline) in the TMJ followed by behavioral assessment as described above (orofacial grooming and resistance to mouth opening). In addition, a third set of mice an receive no treatment and control the injection procedure. All mice can be sacrificed at the end of this 36 hour period and their brain stem and trigeminal ganglia can be harvested for analysis.

b) Effect of Short-Term Brain Stem Neuroinflammation on Neuronal Excitation and Hyperalgesia or Spontaneous Nociception

IL-1β can be administered into the cisterna magna of 2 month old male mice (C57/B16) using a mini-pump via a pediatric catheter over a period of 2 weeks. The osmotic mini-pump can be implanted subdermally in the back of adult mice under surgical anesthesia. The mice can then be evaluated for centrally-induced changes in behavior (spontaneous nociception) as assessed by orofacial grooming and resistance to mouth opening. Comparisons can also be made to the behavioral baseline measurements (prior to IL-1β administration). An additional group of mice can receive an equal volume of sterile saline via the same route of administration and can serve as controls. Moreover, the development of hyperalgesia can be evaluated. A subset of the aforementioned mice can be further challenged by intra-articular injection of formalin (0.625% in saline) in the TMJ followed by behavioral assessment as described above. In addition, another set of mice can receive no treatment and control the injection procedure. Mice sacrificed at each time point can provide their brain stem, trigeminal ganglia and TMJ for analysis.

c) Effect of Long-Term Expression of IL-1β in the Brain Stem on Neuroinflammation and Neuronal Excitation and Behavioral Changes

The long-term effects of neuroinflammation can be evaluated in the brain stem by employing somatic mosaic analysis in the GFAP-IL1β^{XAT} transgenic mouse. In brief, a single injection of the feline immunodeficiency viral vector FIV(Cre) in the intrathecal space activates GFAP-IL1β^{XAT} transgene expression and leads to the development of neuroinflammation at the site of viral transduction. This mouse model offers significant advantages over other models of central nervous system inflammation: It facilitates the development of long-term (several months) neuroinflammation based on the chronic, low level expression of mature and biologically active IL-1β by astrocytes in a temporally and spatially controlled manner. To this end, a single FIV(Cre) injection can be performed in 2 month old GFAP-IL1β^{XAT} transgenic mice under a surgical plane of anesthesia. The mice can then be evaluated for centrally-induced changes in behavior (spontaneous nociception) as assessed by orofacial grooming and resistance to mouth opening Additional mice receiving an equal dose of FIV(lacZ) via the same route of administration can serve as controls. Lastly, mice injected with sterile saline can control for the injection procedure. Comparisons can also be made to the behavioral baseline measurements (prior to IL-1β administration). Moreover, the development of hyperalgesia can be evaluated in a subset of mice further challenged by intra-articular injection of formalin in the TMJ followed by behavioral assessment.

d) Effect of Short-Term IL-1β Neutralization on Pain Processing in the Col1-IL1β^{XAT} Mouse Model of TMJ Arthritis

A neutralizing antibody raised against murine IL1-β (polyclonal; Antigenix America, Huntington St. N.Y.) can be administered over a period of 2 weeks into the cisterna magna of Col1-IL1β^{XAT} transgenic mice that have been previously induced to develop TMJ arthritis using an osmotic mini-pump via a pediatric catheter starting 6 weeks after the FIV(Cre) intra-articular injection. The osmotic mini-pump can be implanted subdermally in the back of adult mice under a surgical plane of anesthesia. The mice can then be evaluated for changes in behavior as assessed by orofacial grooming and resistance to mouth opening. An additional group of mice can receive an equal volume of sterile saline via the same route of administration and can serve as controls. Moreover, the development of hyperalgesia can be evaluated. A subset of the aforementioned mice can be further challenged by intra-articular injection of formalin in the TMJ followed by behavioral assessment.

### 5. Example 5

### The Role of IL-1 Receptor IL1-R1 in the Central Processing of Chronic TMJ Arthritis Pain

IL-1β signaling in the brain stem is important in the processing of orofacial pain, such as in the case of inflammatory pain secondary to chronic TMJ arthritis. IL-1β is known to exert its biological effects via the type 1 receptor (IL1-RI). Thus, as disclosed herein, peripheral inflammatory pain secondary to chronic TMJ arthritis can result in glial cell activation at the trigeminal nuclear complex which in turn causes localized neuroinflammation via the release of inflammatory mediators, in particular IL-1β. Subsequently, IL-1β modulates pain processing at the dorsal horns via the IL-1RI receptor. Disclosed is the evaluation of the role of IL1-RI in the central processing of pain.

a) Experimental Design

The role of IL1-RI in the central processing of inflammatory pain secondary to chronic TMJ arthritis can be evaluated in the Col1-IL1β^{XAT} mouse model, which develops orofacial pain (assessed as behavioral changes and trigeminal sensory excitation) secondary to TMJ arthritis. To this end, three models of IL1-RI receptor inhibition can be employed using the IL-1 receptor antagonist IL1ra. IL1ra is an endogenous antiinflammatory factor found in mammals.

Acute inhibition: This strategy is based on the inhibitory effects of IL1ra administered via a direct injection into the cisterna magna.

Short-term inhibition: This is based on the administration of IL1ra into the cisterna magna over a period of 14 days via a pediatric catheter connected to an implanted osmotic minipump.

Long-term inhibition: a recombinant FIV vector capable of expressing IL-1ra can be employed. In this scenario, a single injection of FIV(IL-1ra) into the cisterna magna results in stable transduction and chronic expression of IL-1ra in the brain stem.

The reason to include three different types of IL1-RI receptor inhibition is based on the need to better understand the functional-temporal relationship of the IL1-RI with pain processing. To this end, long-term inhibition by the FIV(IL1ra) can open new vistas in the management of chronic pain.

b) Effect of Acute Inhibition of the IL1-RI Receptor at the Level of the Brain Stem on the Central Processing of Orofacial Pain Following the Development of TMJ Arthritis

Col1-IL1β^{XAT} mice suffering from orofacial pain secondary to chronic TMJ arthritis can receive a single intrathecal injection of IL1ra into the cisterna magna (10 ng in 2 μL of aqueous solution) under a surgical plane of anesthesia at 8 weeks following induction of TMJ arthritis as described. Thirty six hours later, the mice can be evaluated for changes in behavior (assessed by orofacial grooming and resistance to mouth opening) and comparisons made to baseline measurements (prior to IL1ra injection). An additional group of mice can receive an equal volume of normal sterile saline via the same route of administration and will serve as controls. Moreover, the development of hyperalgesia in a subset mice further challenged by intra-articular injection of formalin in the TMJ can be followed by behavioral assessment.

c) Effect of Inhibition of the IL1-RI Receptor at the Level of the Brain Stem over a Period of 14 days on the Central Processing of Orofacial Pain Following the Development of TMJ Arthritis.

IL1ra will be administered into the cisterna magna (0.25 μl/hr; 5 ng/μl) of Col1-IL1β^{XAT} mice over a period of 2 weeks (from week 6-to-week 8) via a cannula connected to an osmotic mini-pump implanted subdermally in the back of mice as described. At the end of this 2 week inhibition period, changes in behavior can be evaluated (assessed by orofacial grooming and resistance to mouth opening) and comparisons made to baseline measurements (prior to IL1ra administration). An additional group of mice can receive an equal volume of saline via the same route of administration and serve as controls. The development of hyperalgesia can also be evaluated in a subset mice will be further challenged by intra-articular injection of formalin in the TMJ followed by behavioral assessment. All mice can be sacrificed at the end of this experiment and their brain stem and trigeminal ganglia harvested for analysis.

d) Effect of IL-1ra Intracisternal Transduction Using a Lentiviral FIV(IL1ra) Viral Vector on Long-Term Processing of Pain.

Six weeks following induction of TMJ arthritis, Col1-IL1β^{XAT} mice suffering from orofacial pain secondary to chronic TMJ arthritis can receive a single intracisternal FIV(IL1ra) injection (2 μl containing a total of 10⁷ infectious particles/mL) into the cisterna magna. Subsequently, a group of mice can be examined at 8 weeks, a second group at 4 months and a third group at the 6 month time point. At the end of each period, the mice can be evaluated for changes in behavior (assessed by orofacial grooming and resistance to mouth opening) and comparisons made to baseline measurements (prior to IL1ra administration). An additional group of mice can receive an equal dose of FIV(gfp) vector via the same route of administration and serve as controls. In addition, a third set of mice can receive sterile saline and control for the injection procedure. Moreover, the development of hyperalgesia can also be evaluated in these mice. To this end, a subset mice at each time point can be further challenged by intra-articular injection of formalin in the TMJ followed by behavioral assessment as described above. All mice can be sacrificed at the end of each experimental procedure and their brain stem and trigeminal ganglia can be harvested for analysis.

### 6. Example 6

Murine IL-1β (2 ng in 2 μl of normal saline) was injected transdermally in the cisterna magna of deeply anesthetized C57BL/6 mice (anesthetic: ketamine 40 mg/kg IP). Two days later, the mice were sacrificed, transfused transcardially with 4% paraformaldehyde in phosphate buffered saline solution and the brain stem was harvested, frozed and cut at 18 μm thick horizontal sections which were collected on glass slides. The histology slides were then analyzed by immunohistochemistry (IHC) using antibodies raised against calcitonin gene-related peptide (CGRP; μ33) and glial fibrillary acidic protein (GFAP; Dako). Results showed that IL-1β induced the expression of GFAP and CGRP in the descending trigeminal nucleus (medullary dorsal horn) of these mice (FIG. 6).

FIG. 8 shows transgene structure of GFAP-IL1β^{XAT} used to develop transgenic mice. Injection of FIV(Cre) virus in the brain of ROSA26 reporter mice resulted in activation of the reporter gene lacZ in the area of injection.

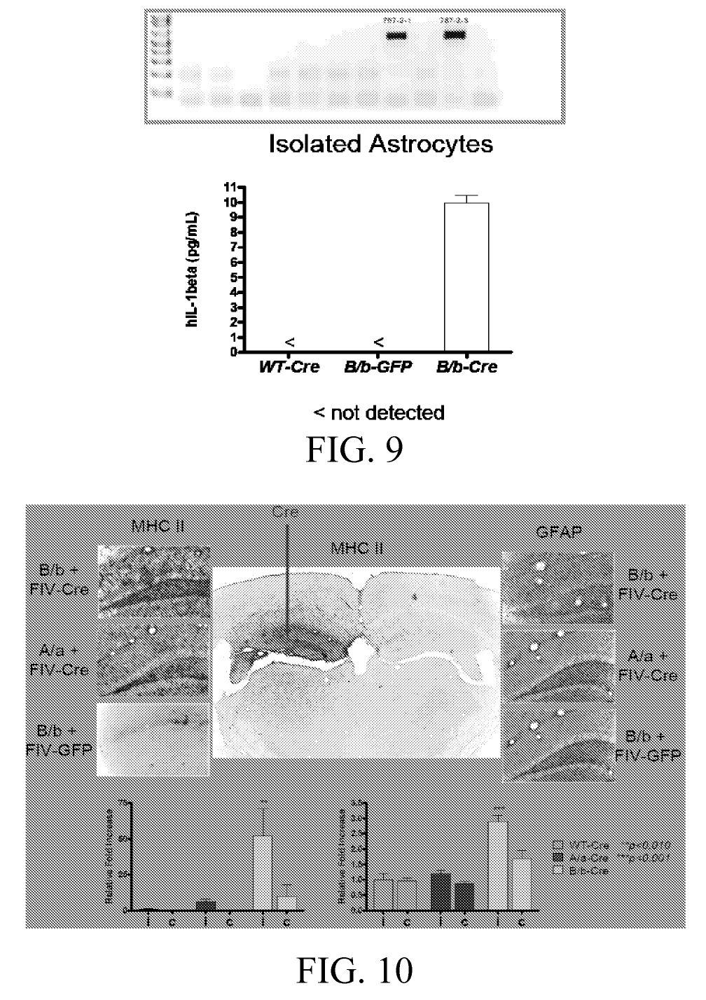

Two transgenic lineswere generated for GFAP-IL1β^{XAT}, namely 787-2-1 (designated as mouse line A) and 787-2-2 (line B). Primary astrocyte cultures from line B were treated with FIV(Cre), which resulted in increased expression of transgenic IL1β as assessed by ELISA (FIG. 9). There was lack of IL1β in the controls (wild type cells treated with Cre or B cells treated with gfp virus) (FIG. 9).

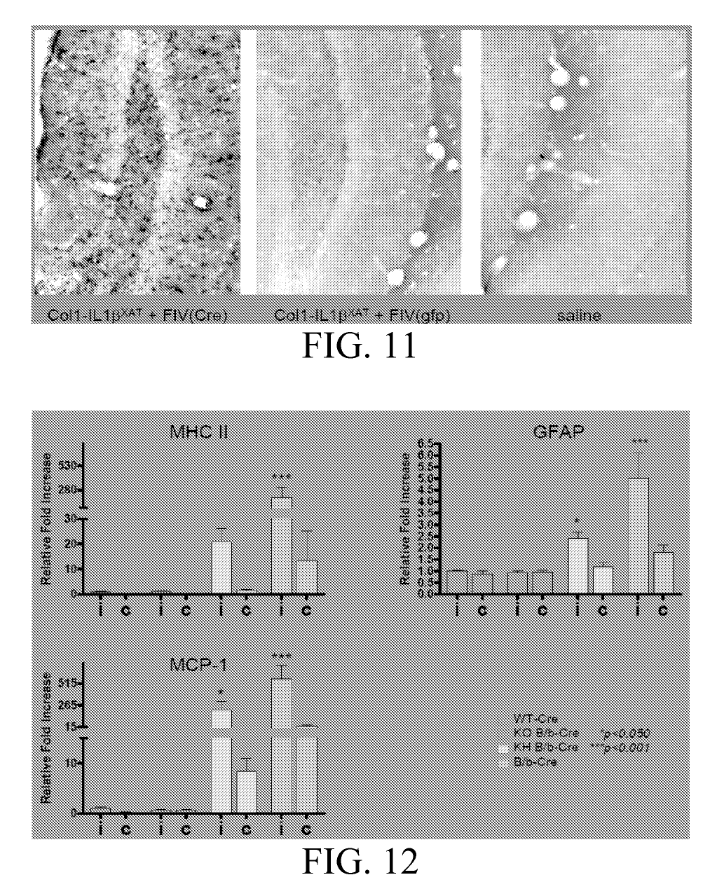

Injection of FIV(Cre) in the brain of B mice resulted in activation of microglia cells, as assessed by major histocompatibility-II (MHC-II) immunohistochemistry (IHC), and astrocyte activation, as assessed by GFAP IHC (FIG. 10). Mouse line A also display induction of these genes but to a lesser degree. FIV(gfp) did not induced any brain inflammation (FIG. 10). Monocyte chemo-attractant protein-1 (MCP-1) was also induced in the B mouse line injected with FIV(Cre) (FIG. 11).

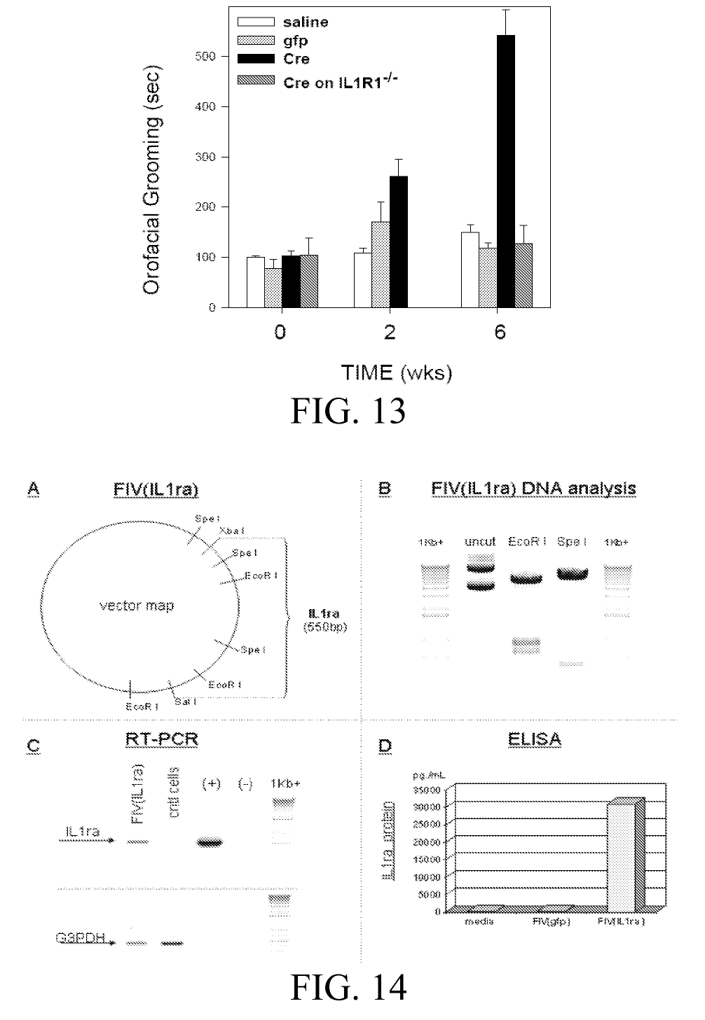

As shown in FIG. 12, the observed inflammation was due to IL-1β induction following FIV(Cre) injection in the GFAP-IL1β^{XAT} transgenic mice. GFAP-IL1β^{XAT} mice were crossed into the IL-1 receptor type 1 (IL1R1^{-/-}) knockout mice and the experiment repeated. Deletion of the IL1R1 in the GFAP-IL1β^{XAT} abolished the previously observed brain inflammation. Injection of FIV(Cre) in the cisterna magna of GFAP-IL1β^{XAT}mice (3 μl of a 10⁶ ip/mL viral stock) resulted in a significant increase (p<0.01) of orofacial pain behavior relative to controls (saline or gfp injection) as assessed by grooming activity at 2 and 6 weeks post injection. Deletion of the IL1R1 gene in these mice abolished their painful behavior (FIG. 13).

As shown in FIG. 14A, shows an FIV(IL1ra) expressing IL-1 receptor antagonist was constructed. Vector sequence was confirmed by multiple restriction enzyme digestions (FIG. 14B). Treatment of 293FT cells with FIV(IL1ra) resulted in induction of IL1ra mRNA as assessed by RT-PCR (FIG. 14C), which also yielded high levels of IL1ra protein in the supernatant media (FIG. 14D). Thus, an IL1 inhibitor such as FIV(IL1ra) can be injected into the cisterna magna of a subject suffering from chronic peripheral pain in order to inhibit the centrally induced pain.

### 7. Example 7

a) Vector Construction & Packaging

The rat neuron specific enolase (NSE) promoter was provided in the pTR-NT3myc-NSE vector. The 2.05 Kb NSE sequence was excised by BglI and HindIII restriction enzyme digestions. The BglI site was blunted by T4 DNA polymerase and the fragment was subsequently cloned into the Xho I (blunt)-Hind III (sticky) sites of the pBluescript II KS+/−phagemid forming pBS-NSE. The human μ-opioid receptor (HuMOR) cDNA was provided in the pcDNA3 plasmid. The 1.6 Kb HuMOR sequence was excised by EcoRV and XbaI digestions and cloned into the EcoRV-XbaI sites of pBS-NSE to form pBS(NSE-HuMOR). Subsequently, the HuMOR cDNA was cloned by blunt-sticky ligation into the Hind III (blunt)-Xba I (sticky) sites of pRc/CMV (Invitrogen, Carlsbad Calif.) expression vector for transient expression experiments. In addition, the Kpn I (blunt)-Xba I (sticky) NSE-HuMOR (3.65 Kb) fragment was cloned into the Nru I (blunt)-Xba I (sticky) sites of the pRc/CMV expression vector by excising the vector's CMV promoter.

The NSE-HuMOR fragment was also cloned into the Lenti6 Lentiviral Expression System (ViraPower™; Invitrogen) following a modification of the vector's cloning site. Specifically, using the 5′CACCTAATACGACTCACTATAGG3′ (SEQ ID NO. 41) and 5′CATTAACCCTCACTAAAG3′ (SEQ ID NO. 42) primer set a 707 bp fragment was PCR amplified out of the pIRES vector's multiple cloning site (Clontech). The upper primer contained the CACC sequence which assisted in the fragment's directional topoisomerase-mediated cloning into the pLenti6/V5-D-TOPO vector according to manufacturer's instructions, creating the new LV lentiviral vector with the desired Nhe I-Sal I sites. The CMV promoter was then removed by Cla I and Spe I restriction enzyme digestions, the ends were blunted and the vector was re-circularized. In order to clone NSE-HuMOR into the LV vector, the pBS(NSE-HuMOR) was digested with Kpn I (blunt)-Xba I (sticky) and cloned into the EcoR I (blunt)-Xba I (sticky) sites of the pIRES vector. Subsequently, a Nhe I-Sal I pIRES fragment containing NSE-HuMOR was cloned into the Nhe I-Sal I sites of the LV vector creating LV(NSE-HuMOR).

For the FIV(CMV-HuMOR) construction, the HuMOR cDNA was excised from the pcDNA3 plasmid by Hind III digestion and cloned into the Hind III site of the pBS vector in the desired 5′-3′ orientation. Subsequently, the Xba I-Sal I segment containing HuMOR was excised from the pBS vector and cloned into to the commercially available FIV(LacZ) vector (Systems Biosciences; Mountain View, Calif.) between Xba I-Sal I sites in place of the lacZ gene.

FIV vectors were packaged in 293-FT cells (Invitrogen) cultured in T75 flasks, which were grown to subconfluency in DMEM plus 10%; FBS (Gemini, Woodland, Calif.). The cells were then co-transfected with the transfer vector, LV(NSE-HuMOR) or FIV(HuMOR), the packaging (Poeschla 1998) and the VSV-G pseudotyping vectors (Burns 1993) using the Lipofectamine 2000 reagent (Invitrogen) per manufacturer's instructions. Twenty-four hours after transfection, the supernatant medium was discarded and replaced by fresh medium. Sixty hours after transfection, the virus-rich supernatant was collected, filtered through 0.45 mm SurfilR-MF filter (Corning Seperations Division, Acton Mass.) and subsequently concentrated by overnight centrifugation at 7000 g using a Sorvall RCSB high-speed centrifuge and a SLA-3000 rotor. Subsequently, the supernatant was decanted, and the viral pellet was resuspended overnight in 1 mL of normal buffered saline containing 40 mg/mL lactose at 4° C. The viral solution was then aliquoted and frozen (−80° C.) until further use. Titering was performed on CrfK cells (American Tissue Culture Collection, Manassas, Va.) cultured in 24 well tissue culture plates. Specifically, during packaging, LV(lacZ) or FIV(lacZ) was added in the mix at a 1:100 ratio to the respective transfer vector. Titers were calculated based on the number of X-gal positive cells and extrapolated based on the dilution factor. Titers routinely range between 107-108 infectious particles/mL.

b) In Vitro Studies

The pRc/CMV-HuMOR and pRc/NSE-HuMOR plasmids were transfected into 293FT and N2α cells, respectively, using the Lipofectamine 2000 reagent per manufacturer's instructions (Invitrogen). Forty-eight hours later, total RNA was extracted using the TRIzol reagent (Invitrogen) and HuMOR mRNA levels were assessed by RT-PCR using the 5′GAATTACCTAATGGGAACATGG3′(SEQ ID NO:45) and 5′GCAGACGATGAACACAGC3′ (SEQ ID NO:46) primers set (TA=56° C., 30 cycles). The G3PDH house keeping gene transcript levels were evaluated using the 5′ACCACAGTCCATGCCATCAC3′ (SEQ ID NO:55) and 5′TCCACCACCCTGTTGCTGTA3′ (SEQ ID NO:56) primers set (TA=58° C., 30 cycles). NSE-HuMOR expression in N2α cells was also evaluated by immunohistochemistry (IHC) employing a rabbit anti-HuMOR IgG antibody (1:1,000 dilution) commercially available from Chemicon (AB1580; Temecula, Calif.). In brief, the cells were washed with phosphate buffered saline (PBS), fixed with 10% paraformaldehyde for 15 min, rinsed with PBS, blocked with 4% normal goat serum (NGS) in PBS and incubated for 60 min in primary antibody solution containing 0.4% Triton-X and 4% NGS at room temp. The cells were then washed with PBS and blocked again in 4% NGS following by secondary antibody incubation for 60 min at room temp. The cells were then washed with PBS and incubated in ABC solution (Vector Laboratories, Burlingame VM) for 60 min followed by incubation in DAB solution for 4 min for visualization of immunoreactivity (brown staining). N2α cells were also infected with LV(NSE-HuMOR) or LV(lacZ) at m.o.i.˜2 in vitro and total RNA was harvested 60 hrs later using the TRIzol reagent (Invitrogen) per manufacturer's instructions. HuMOR expression was evaluated by the aforementioned RT-PCR protocol.

c) Animal Studies

All animal procedures described were reviewed and approved by the Institutional Animal Care and Use Committee (University Committee on Animal Resources) for compliance with federal regulations prior to the initiation of the study (OLAW/PHS Assurance A3292-01). All mice were maintained in an AAALAC-accredited specific pathogen free barrier facility. All procedures followed the AVMA guide per institutional policy.

Two month old C57B16 male wild type mice were injected intra-articularly with 50 μl of LV(NSE-HuMOR) or LV(lacZ) in the right TMJ: A total of 5×10⁵ infectious particles/mL were injected in each joint. In brief, the mice were anesthetized by ketamine (40 mg/Kg) and under surgical plane of anesthesia the right TMJ was located by palpation over the zygomatic arch from an anterior to posterior direction. A 27½ G needle was inserted in a posterior-inferior direction and solutions were injected into the superior joint space. After injection, the mice were returned to their cages. Five weeks later, the mice were euthanized and the right side trigeminal ganglia were harvested and analyzed by as follows. The presence of HuMOR was assessed by PCR in DNA extracts from the ganglia using the DNAzol reagent (Invitrogen) per manufacturer's instructions. HuMOR expression was evaluated by RT-PCR in total RNA extracts from the ganglia using the TRIzol reagent (Invitrogen) per manufacturer's instructions.

Three month old Col1-IL-1β^{XAT} mice were injected with 50 μl containing 1×10⁶ FIV(HuMOR) infectious particles in the right and left TMJ under surgical plane of anesthesia as described above. One week later, the mice received a second intra-articular injection of 50 μl containing 5×10⁶ FIV(Cre) infectious particles in both TMJs under surgical plane of anesthesia and returned to their cages. Mouse behavior was subsequently evaluated every two weeks and finally sacrificed 8 weeks following the FIV(Cre) injection.

Grooming behavior was evaluated by adapting a method previously described (Lai 2006). In brief, mice were placed in a custom-made cage (12″×12″×12″) with 4 mirrored walls. The cage lacked a roof so that the mice could be observed and recorded. Each mouse was transferred into the aforementioned observation chamber containing bedding from its original cage and was allowed a 30 min habituation period to minimize stress. Behaviors were recorded on a video-tape for a period of 60 minutes using a Sony digital recorder (Digital Handycam/Digital 8) with a Cokin macro digital lens (mode C043) added for image enlargement. The mouse was then returned to its original cage. Grooming was measured during play-back by counting the number of seconds a mouse rubbed its face and/or flinched its head during the session by a single observer. The mice did not have access to food or water during the brief testing period. Behavioral evaluation was performed by an investigator blinded to the mouse group assignment. The behavior was characterized in 3 minute increments over the 60 minutes of evaluation. These data were entered into FileMaker Pro V7 (FileMaker Inc.; Santa Clara, Calif.) and exported to Excel (Microsoft Inc.) for analysis.

Resistance to jaw opening was employed as a method for assessing temporomandibular joint dysfunction based on the principles of the Pain Adaptation Model (Lund et al. 1991), which suggests that pain reduces muscle force. In the morning and in preparation for the test, the mice were anesthetized via intra-peritoneal injection of ketamine (40 mg/Kg). An orthodontic hook was attached using dental bonding material onto the lower incisors and the mouse was returned to its cage to recover from anesthesia for a minimum of 4 hours. The evaluation resumed in the afternoon of the same day. Each mouse was then placed in a plastic (single use) restraining device which immobilizes the head and the maxilla while leaving the mandible free. The lower jaw was then connected via the orthodontic hook to a digital dynamometer (FGF series, Kernco Instruments) wired to a DELL PC computer through an A/D conversion card (NIO16E1, National Instruments) which recorded the resistance exhibited by the mouse during an attempt to displace the mandible vertically by 4 mm. A total of 10,000 data points over approximately 220 seconds were collected by the Lab View software package (National Instruments, Austin Tex.) on a PC computer and plotted over a 5 min time period. Within each period the mandible was lowered 10 times and held for approximately 2 seconds, with a 20 second wait between each depression of the mandible. At the end of each session, the mice were sacrificed.

d) Histological—Immunohistochemical Studies

Following fixation in 10% formalin, the mouse heads were dissected, de-fleshed and decalcified by immersion in an EDTA solution for 7 days in 4° C. under constant agitation. The TMJs were then processed on a RHS-1 microwave tissue processor, after which the samples were embedded in paraffin, cut on a microtome as 3 μm thick sections and collected on glass slides. The brain stem and ganglia were cut frozen on a cryostat as 18 μm thick sections and collected on glass slides. Overall TMJ histopathology was evaluated in sections stained by Alcian blue-orange G histochemistry using a scale 0-4 previously described by Lai et al. (2006). This scale is defined as follows: “0=no apparent changes; 1=superficial fibrillation, striation of cartilage; 2=injuries limited to uncalcified cartilage; 3=Defects extending into calcified cartilage; 4=deep defects extending into calcified cartilage. Articular chondrocyte cloning was assessed by counting the number of lacunae containing more than one chondrocytes in the articular cartilage.

Immunohistochemical (IHC) analysis was performed for a number of antigens using antibodies described below. In general, brain stem and ganglia sections were rehydrated in PBS for 60 min, bleached in 3% H₂O₂ for 15 min and processed as follows. Tissues were blocked using appropriate primary serum solution followed by overnight incubation in primary antibody solution at 4° C. The following morning, the TMJ sections were rinsed with PBS and incubated in an appropriate biotinylated secondary antibody solution for 30 min, followed by PBS wash and incubation in horseradish peroxidase-conjugated streptavidin. AEC was employed as chromagen and sections were counterstained with hematoxylin, followed by PBS wash. Brain stem and ganglia sections were processed in a similar fashion except that the ABC reagent (Vector Laboratories, Burlingame Calif.) was used in conjunction with Nickel—DAB as chromagen as previously described (Kyrkanides et al. 2004). The sections were then dehydrated in alcohols, cleared by xylene and cover-slipped with permanent mounting media. The histology sections were evaluated under light microscopy using an Olympus BX51 microscope. Microphotographs were captured using a Spot CCD digital camera attached to the microscope. The TMJ sections were deparaffinized in xylene, rehydrated through graded alcohols and quenched in 3% H₂O₂ for 20 min. Antigen retrieval was performed in a pressure cooker using a 10 mM citrate buffer pH 6.0 at 90° C. for 15 min. Antibodies used in these experiments include a rabbit anti-HuMOR antibody (AB1580, 1:1,000 dilution; Chemicon, Temecula, Calif.), a rabbit anti-c-Fos antibody (SC-52, 1:3,000 dilution; Santa Cruz Biotechnology Inc, Santa Cruz Calif.), a rabbit anti-murine IL-1β antibody (RMF 120, 1:1,000 dilution; Antigenix America, Huntington Station, N.Y.) and a rabbit anti-GFAP antibody (Z0334, 1:1,000 dilution; Dako, Carpinteria, Calif.). GFAP immunoreactivity was measured in the brain stem and ganglia sections as the number of immunoreactive pixels per in each microscopic field (10×) by the NIH image software program (Lai et al. 2006). The number of c-Fos+ and IL-1β+ cells were counted in each microscopic field by one investigator (SK).

e) Results

(1) HuMOR Expression in Mammalian Cells

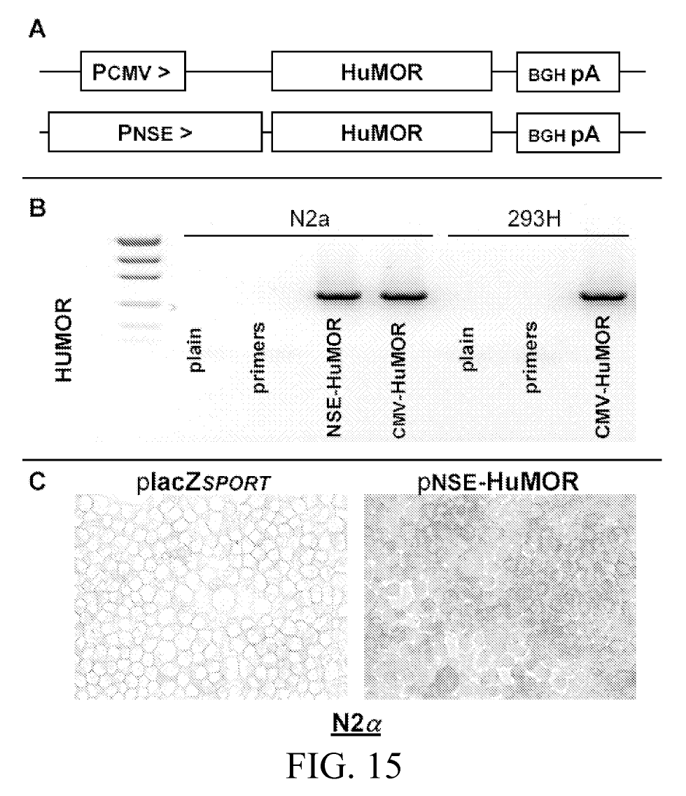

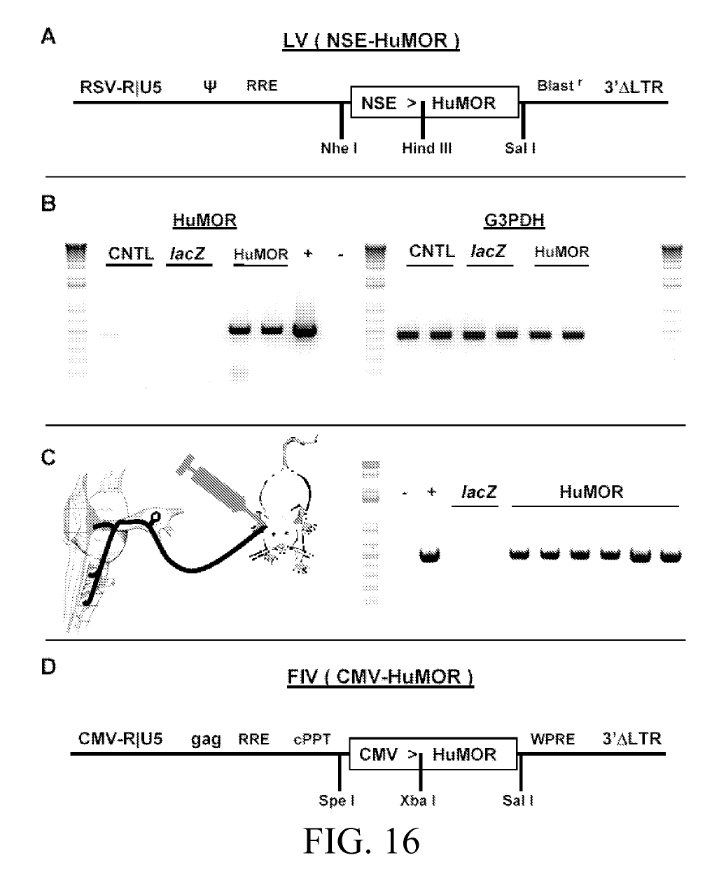

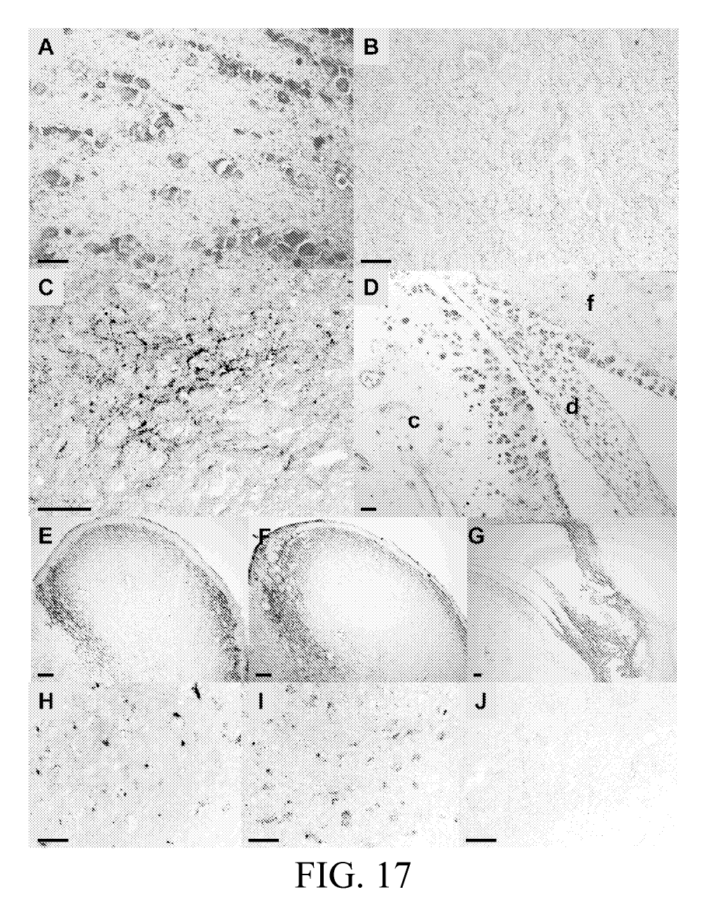

HuMOR overexpression was targeted in the human-derived N2α neuronal cell line by the NSE promoter, as well as in the human-derived 293FT fibroblast cell line by the CMV promoter in vitro (FIG. 15). NSE-HuMOR overexpression was also detected by immunohistochemistry in N2α cells, which also displayed a modest background level of HuMOR expression at naïve conditions (FIG. 15C). N2α cells were successfully infected using the LV(NSE-HuMOR) at m.o.i.˜2, a recombinant lentiviral vector system (FIG. 16A), as assessed by the increased HuMOR mRNA levels (FIG. 16B). As expected, the control vector LV(lacZ), previously shown to transduce mammalian cells with the reporter β-galactosidase gene, did not induce HuMOR expression in the N2α cells. In vivo, LV(NSE-HuMOR) was injected intra-articularly into the temporomandibular joint (TMJ) of wild type mice and the vector was traced in the ipsilateral trigeminal ganglia (FIG. 16C). Specifically, the transduction of primary sensory neurons innervating the TMJ was demonstrated by the presence of HuMOR transgene in DNA extracts of trigeminal ganglia 5 weeks post-transduction by PCR using primers that distinguish between the human and the mouse μ-opioid receptor nucleotide sequence. Interestingly, HuMOR expression was not detected in these ganglia, as evaluated by RT-PCR, despite the presence of the transgene in the ganglia, presumably due to a low number of HuMOR transcript copies and/or limited infection LV efficacy. These results prompted the construction of FIV(CMV-HuMOR), a lentiviral vector platform (FIG. 16D) shown to effectively transduce trigeminal sensory neurons following intra-articular injections (Kyrkanides et al. 2004).

(2) Trigeminal Sensory Neuron Transduction by a Recombinant Feline Immunodeficiency Virus

A total of 1×10⁶ infectious FIV(CMV-HuMOR) particles contained in 50 μl of aqueous solution were injected bilaterally into the TMJ joint space of young adult Col1-IL-1B^{XAT} transgenic mice. A week later, these mice received a second intra-articular injection containing a total of 5×10⁶ infectious particles of FIV(Cre) to induce transgene activation and arthritis in the TMJ as previously described (Lai 2006). Subsequently, HuMOR expression was evaluated 8 weeks later in the trigeminal ganglia by IHC. HuMOR immunoreactive cells were observed in trigeminal ganglion (FIG. 17A), located in an area of this sensory ganglion previously implicated in the innervation of the TMJ (Kyrkanides 2004). In the brainstem, HuMOR immunopositive fibers were primarily observed in the trigeminal subnucleus caudalis (FIG. 17C) with only a few fibers present at the level of the main sensory nucleus. These fibers represent proximal branches of sensory trigeminal fibers transduced peripherally by FIV(HuMOR). In addition, HuMOR immunoreactivity were observed in the hard and soft tissues of the TMJ, including articular fibrocartilage and joint meniscus (FIG. 17D). Interestingly, μ-opioid receptor ligands, including met-enkephalin and leu-nekephalin, were immunolocalized in the subnucleus caudalis (FIG. 17E-17F) but not in the main sensory nucleus, indicating this nucleus as an area important in the central processing of nociception from the TMJ (pain control). In addition, enkephalins were also immunolocalized in the TMJ, particularly in synovial tissue primarily of the posterior and to a lesser degree anterior meniscal attachment (FIG. 17G). The anatomical link between the TMJ and brain stem nuclei was confirmed by retrograde tracing experiments using DiI tracer. Intra-articular administration of the tracer in the right TMJ led to the identification of DiI fluorescence in the subnucleus caudalis (FIG. 17H) as well as in the main trigeminal sensory nucleus (FIG. 17I), demonstrating a direct connection of these structures via the Vth cranial nerve.

(3) Induction of HuMOR in the TMJ Modifies Pain Behavior and Attenuates Joint Pathology

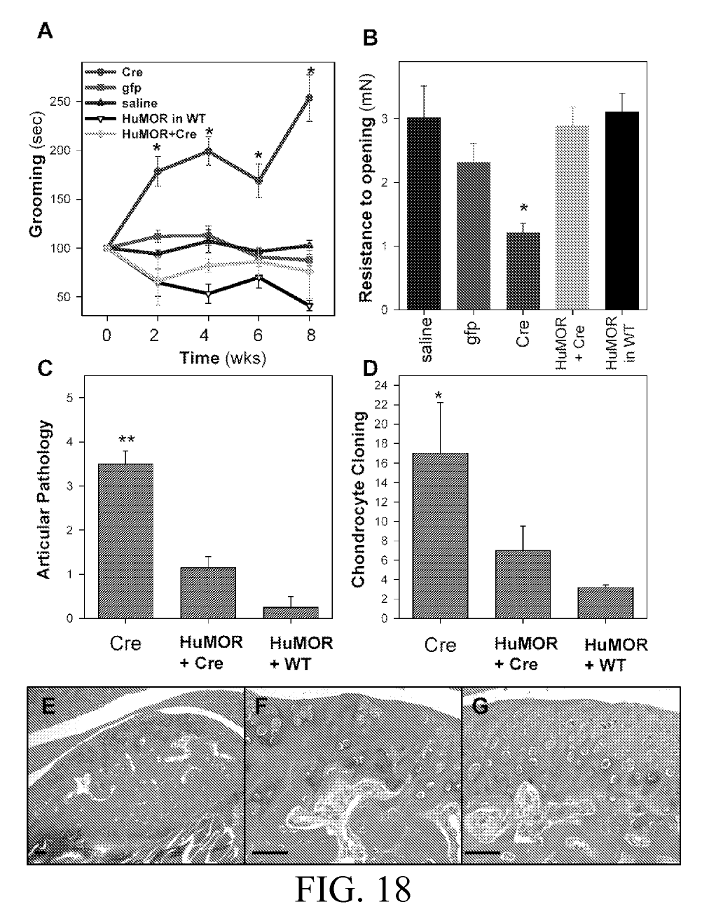

Intra-articular FIV(HuMOR) injection in the TMJ prior to the initiation of arthritis in the Col1-IL1β^{XAT} mouse model significantly attenuated orofacial pain behavior as evaluated by a reduction in orofacial grooming activity (FIG. 18A). Moreover, FIV(HuMOR) pre-treatment reduced joint dysfunction as measured by resistance to mouth opening (FIG. 18B). Interestingly, FIV(HuMOR) pre-treatment significantly attenuated the development of joint pathology and reduced chondrocyte cloning in arthritic mice. To this end, FIV(HuMOR) also transduced articular chondrocytes and meniscal tissues, mediating to some degree the observed attenuation of joint arthritis (FIGS. 18C & 18D). FIV(HuMOR) intra-articular administration did not affect behavior (FIG. 18A) and had no detectable effect on joint anatomy in wild type controls (FIGS. 18C, 18D & 18G). In conclusion, FIV(HuMOR) pre-treatment ameliorates orofacial pain and joint dysfunction in animals suffering from TMJ arthritis and is associated with a reduction in the degree of joint pathology.

(4) Orofacial Pain and Brain Stem Activity

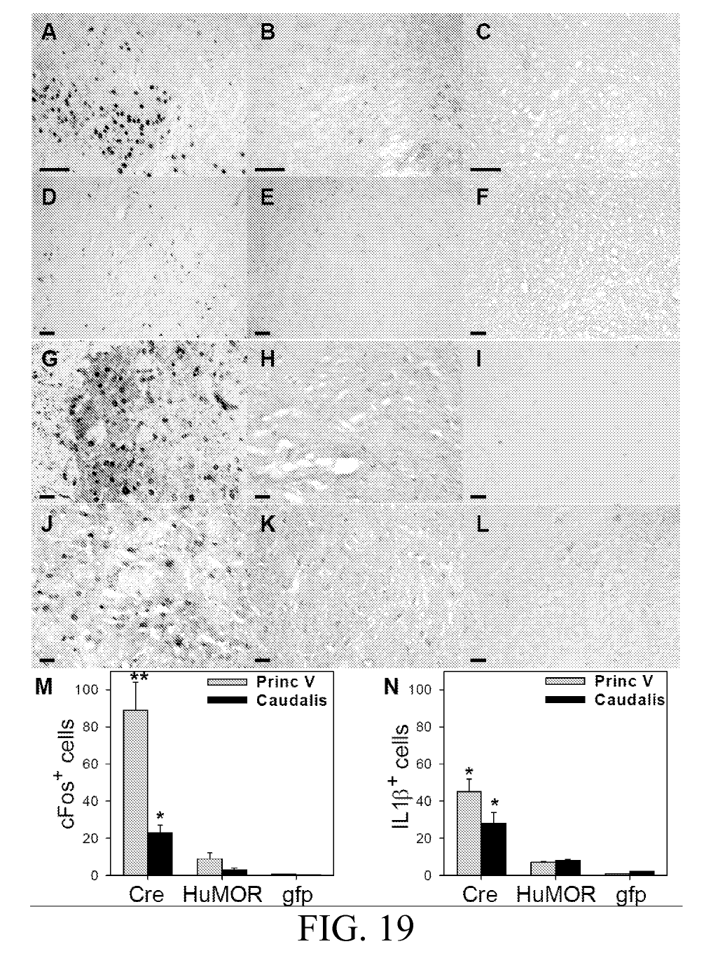

The induction of TMJ arthritis, dysfunction and pain in the Col1-IL-1β^{XAT} mouse model was accompanied by increased c-Fos immunoreactivity in the trigeminal sensory nuclei located in the brain stem, a marker of neuronal activation associated with hyperlagesia and pain. Specifically, a significant increase in the number of c-Fos immunopositive cells was observed in the trigeminal subnucleus caudalis as well as main sensory nucleus. In addition, a significant increase in the number of cells expressing murine IL-1β was observed at these nuclei in Col1-IL1β^{XAT} arthritic mice compared to controls (FIG. 19A-19F). IL-1β expression was mainly observed at the level of the subnucleus caudalis as well as main sensory nucleus. Central IL-1β was previously associated with conditions of hyperalgesia and pain and was employed here as a marker of such (Oka 1995; Sommer 1999). Moreover, FIV(HuMOR) pre-treatment normalized this central IL-1β expression in arthritic mice to levels comparable to control mice. These data demonstrate activation of secondary sensory neurons participating in the central processing of TMJ nociception as well as implicate a central role for IL-1β in this process.

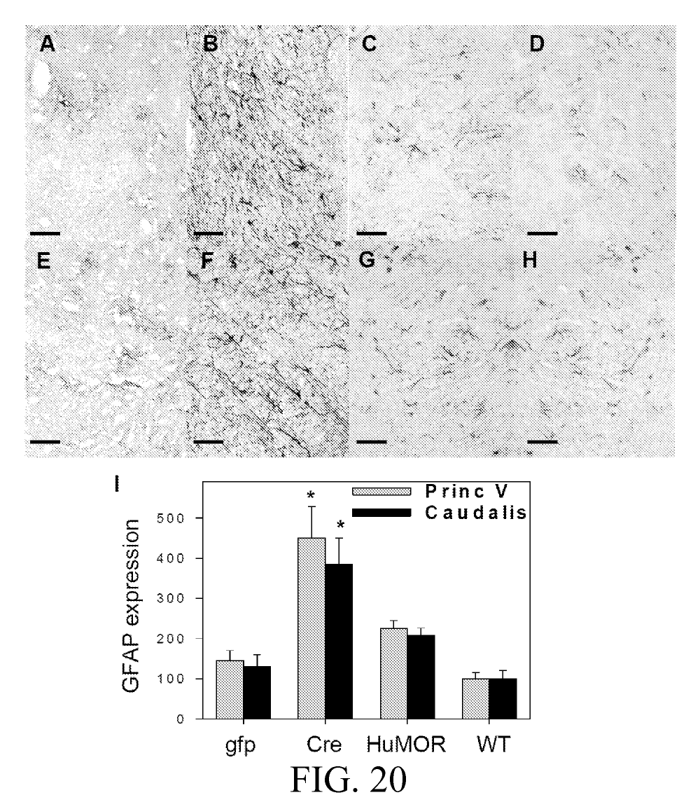

A significant level of astroglia activation, as evaluated by GFAP immunohistochemistry, was also noted in the subnucleus caudalis and main sensory nucleus of Col1-IL1β^{XAT} arthritic mice compared to controls (FIG. 20). Moreover, FIV(HuMOR) pre-treatment attenuated this GFAP induction to levels comparable to those observed in control mice. There was lack of Mac-1 (CD11b+) immunoreactivity in these sections, a marker of activated microglia (or infiltrating monocytes). These data demonstrate that astroglia are activated in conjunction to orofacial/TMJ pain at the level of the subnucleus caudalis and main sensory nucleus, a process that is mediated by sensory afferent fibers and can be modulated by the opioid system.

### 8. Example 8

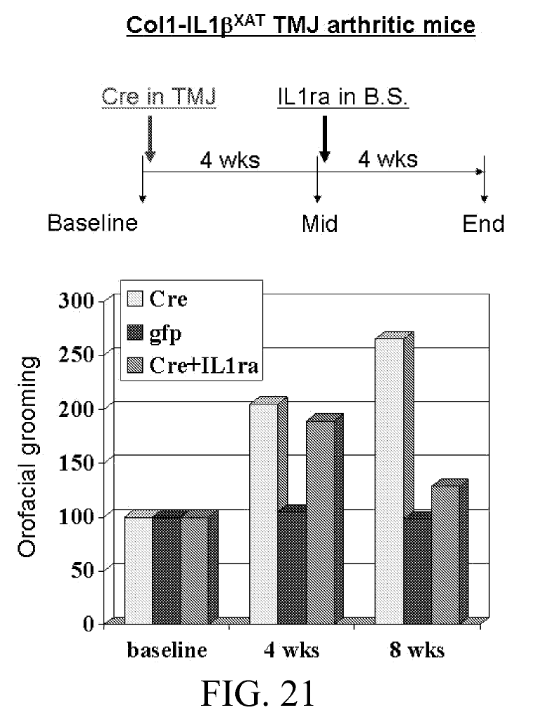

Col1-IL1β^{XAT} mice that were injected in the TMJ with Cre vector began showing signs of orofacial nociceptive behavior 4 weeks following the TMJ injection (FIG. 21). Subsequently, FIV(IL1ra) was then administered to a subset of these mice via a single injection into the cisterna magna (3 μl containing 1.5×10⁶ infectious particles). The mice were then returned to their cages. At the end of the (8 week) experiment, all mice were evaluated. The group of mice with TMJ arthritis that were injected with FIV(IL1ra) in the cisterna magna displayed amelioration of the nociceptive behavior (FIG. 21). Conversely, nociceptive behavior increased in the mice without IL1ra treatment (FIG. 21). As control, Col1-IL1β^{XAT} mice that were injected with the control gfp vector did not display any signs of nociceptive behavior at the 4 or 8 week time point (FIG. 21).

These data demonstrate that activation of the IL1β-IL1RI signaling pathway in the brain stem is necessary for the development of orofacial nociceptive behavior in mice suffering from TMJ arthritis. Moreover, inhibition of the IL1RI receptor with IL1ra (or other similar compounds) can provide a basis for the development of new therapies for orofacial pain.

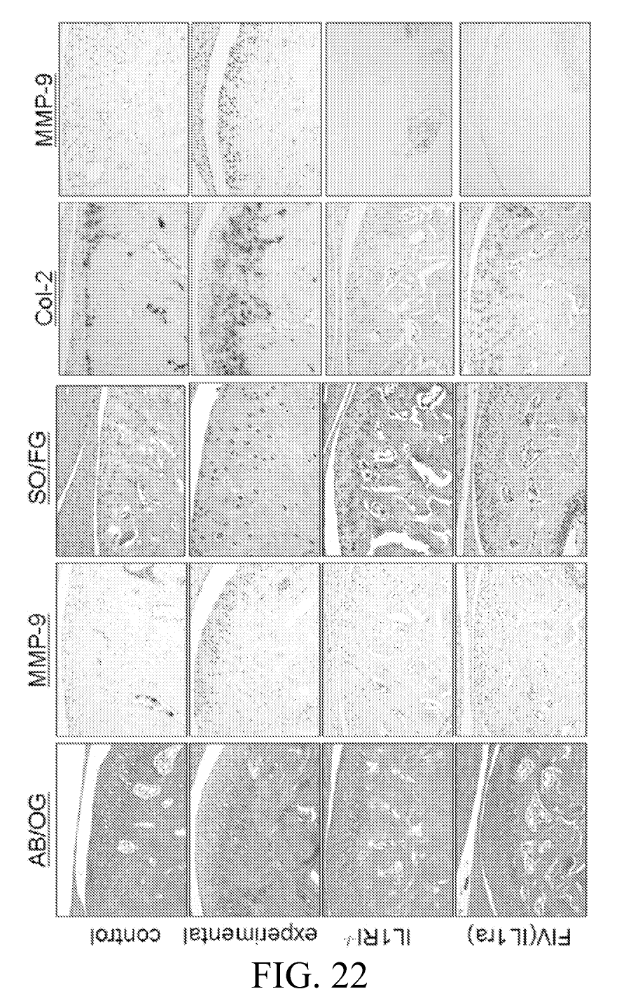

Alcian blue histochemistry (AB/OG), MMP-9 immunohistochemistry (MMP-9), acidic proteoglycans (SO/FG), and type II collagen immunohistochemistry (Col-2) were employed in the histopathological evaluation of the TMJ in the following mouse groups: Control—GFAP-IL1β^{XAT} Tg mice injected with FIV(gfp) in the cisterna magna (brain stem); Experimental—GFAP-IL1β^{XAT} Tg mice injected with FIV(Cre) in the cisterna magna; IL1R1^{-/-}—GFAP-IL1β^{XAT};IL1RI^{-/-} compound mice injected with FIV(Cre) in the cisterna magna; FIV(IL1ra)—Col1-IL1β^{XAT} Tg mice that were injected with FIV(Cre) in the TMJ and followed with FIV(IL1ra) injection into the cisterna magna (FIGS. 22A and 22B).

These data demonstrate that (1) central induction of IL1β expression in the brain stem of mice results histological changes in the TMJ: reduction in cartilage content in the superficial cartilage layers (AB/OG); (2) upregulation of MMP-9 and IL-6, classic markers on joint arthritis; (3) a decrease in proteoglycant content (SO/FG); (4) Induction of Col-2 expression usually seen in the initial stages of osteoarthritis.

Deletion of the IL1RI receptor in the GFAP-IL1β^{XAT} Tg mouse model rescued the mice from developing the aforementioned pathology (IL1RI^{-/}-group). To this end, inhibition of the IL1RI receptor in the brain stem of Col1-IL1β^{XAT} Tg mice suffering from (peripherally induced) arthritis in the TMJ (see Lai et al. 2005) resulted in amelioration of the TMJ pathology.

### G. References

Abbott F V, Franklin K B J, Conel B (1986). The stress of a novel environment reduces formalin pain: possible role of serotonin. Eur J Pharmacol 126: 126-41.
Abramowitz M and Stegun I A (1964). Handbook of Mathematical Functions, Applied Mathematics Series, vol. 55 (Washington: National Bureau of Standards; reprinted 1968 by Dover Publications, New York).
Abreu, S., Hayden, J., Berthold, P., Shapiro, I. M., Decker, S., Patterson, D., & Haskins, M. (1995) Calc. Tissue Intl. 57,185-190.
Adamo C T, Mailhot J M, Smith A K, Borke J L (2001) Connexin 43 expression in oral derived human osteoblasts after transforming growth factor-beta and PGE2 exposure. J Oral Implantol 27: 25-31
Agarwal S, Long P, Gassner R, Piesco N P, Buckley M J (2001). Cyclic tensile strain suppresses catabolic effects of interleukin-1beta in fibrochondrocytes from the temporomandibular joint. Arthr Rheum 44: 608-17.
Ahmadzadeh N, Shingu M, Nobunaga M (1990). The effect of recombinant tumor necrosis factor-alpha on superoxide and metalloproteinase production by synovial cell and chondrocytes. Clin Exp Rheumatol 8:387-91.
Alisky J M, Hughes S M, Sauter S L, Joly D, Dubensky Jr. T W, Staber, P D, et al. (2002) Transduction of murine cerebellar neurons with recombinant FIV and AAV5 vectors. Mol Neurosci 11: 2669-2673.
Anderson G D, Hauser S D, McGarity K L, Bremer M E, Isakson P C and Gregory S A (1996). Selective inhibition of cyclooxygenase (COX)-2 reverses inflammation and expression of COX-2 and interleukin 6 in rat adjuvant arthritis. J Clin Invest 97:2672-79.
Balkhi K M, Tallents R H, Hutta J, Murphy W, Proskin H (1993). Electromyographic Evaluation of the
Baum B J, Kok M, Tran S D, Yamano S (2002). The impact of gene therapy on dentistry: a revisiting after six years. JADA 133: 35-44.
Bellinger D L, Felten D L, Lorton D. Brouxhon S M (2001). Effects of interleukin-2 on the expression of corticotropin-releasing hormone in nerves and lymphoid cells in secondary lymphoid organs from the Fischer 344 rat. J Neuroimmunol 119:37-50.
Bernick S (1962). The vascular and nerve supply to the temporomandibular joint of the rat. Oral Surg 15:488-492.
Biddulph, D. M., Dozier, M. M., & Capehart, A. A. (2000). Methods Cell. Sci. 22, 9-16.
Biddulph, D. M., Dozier, M. M., Julian, N. C., & Sawyer, L. M. (1991) Cell Differ. Dev. 25, 65-75.
Bloemer U, Naldini L, Kafri T, Trono D, Verma I M, Gage F H (1997). Highly efficient and sustained gene transfer in adult neurons with a lentivirus vector. J Virol 71: 6641-6649.
Breyer R M, Bagdassarian C K, Myers S A and Breyer M D (2001). Prostanoid receptors: subtypies and signaling. Ann Rev Pharmacol Toxicol 41:661-90.
Brooks A I, Halterman M W, Federoff H J (1999). Focal hippocampal gain of NGF function elicits specific septal cholinergic reorganization. Neuroreport. 10:337-44.
Brooks A I, Muhkerjee B, Panahian N, Cory-Slechta D, Federoff H J (1997). Nerve growth factor somatic mosaicism produced by herpes virus-directed expression of cre recombinase. Nat Biotech 15(1):57-62
Brummelkamp, T. R., R. Bernardsand R. Agami. A system for stable expression of short interfering RNAs in mammalian cells. Science (2002) 296:550-553.
Bullough P G, DiCarlo E F (1990). Subchondral avascular necrosis: a common cause of arthritis. Ann Rheum Dis 49:412-20
Burkhardt H, Schwingel M, Menninger H, Macartney H W, Tschesche H (1986). Oxygen radicals as effectors of cartilage destruction. Direct degradative effect on matrix components and indirect action via activation of latent collagenase from polymorphonuclear leukocytes. Arthritis Rheum 29:379-87.
Burns J C, Fiedmann T, Driever W, Burrascanno M, Yee J-K (1993). Vesicular stomatitis virus G glycoprotein pseudotyped retroviral vectors: Concentration to very high titer and efficient gene transfer into mammalian and nonmammalian cells. Proc Natl Acad Sci USA 90: 8033-8037.
Calvelou P, Dallel R, Orliaguet T, Woda A (1995). The orofacial formalin test in rats: effects of different formalin concentrations. Pain 62:295-301.
Capehart, A. A., & Biddulph, D. M. (1991) J. Cell. Physiol. 147, 403-411.
Capra N F (1987). Localization and central projections of primary afferent neurons that innervate the temporomandibular joint in cats. Somatosensory Res 4: 201-213.
Carleson J, Alstergren P, Appelgren A, Appelgren B, Kopp S, Theodorsson E, et al. (1996). A model for the study of experimentally induced temporomandibular arthritis in rats: the effect of human recombinant interleukin-I alpha on neuropeptide like immunoreactivity. J Orofac Pain 10:9-14.
Carneiro F A, Bianconi L, Weissmueller G, Stauffer F, Da Poian A T (2002). Membrane recognition by vesicular stomatitis virus involves enthalpy-driven protein-lipid interactions. J Virol 76: 3756-3764.
Chandrasekharan N V, Dai H, Roos K L, Evanson N K, Tomsik J, Elton T S, Simmons D L (2002). COX-3, a cyclooxygenase-1 variant inhibited by acetaminophen and other analgesic/antipyretic drugs: cloning, structure, and expression. Proc Natl Acad Sci USA. 99:13926-31.
Chang J S, Gillman S C, Lewis A J (1986). Interleukin 1 activates phospholipase A2 in rabbit chondrocytes: a possible signal for IL-1 action. J Immunol 136:1283-87.
Chepenik, K. P., Ho, W. C., Waite, B. M., & Parker, C. L. (1984) Calc. Tissue Int. 36, 175-181.
Choi H S, Lee H J, Juan C Y, Ju J S, Park J S, Aim D K (2003). Central cyclooxygenase-2 participates in interleukin-β-induced hyperalgesia in the orofacial formalin test of freely moving rats. Neurosci Letters 352: 187-90.
Cox B M, Ginsburg M, Osman O H (1968). Acute tolerance to narcotic drugs in rats. Br J Pharmacol 245-256.
Crombie I K, Croft P R, Linton S J, LeResche L, Von Korff M (eds). Epidemiology of Pain: A report of the Task Force on epidemiology of the International Association for the Study of Pain. Seattle: IASP, 1999.
Daly T M, Vogler C, Levy B, Haskins M E, Sands M S (1999) Neonatal gene transfer leads to widespread correction of pathology in a murine model of lysosomal disease. Proc Natl Acad Sci USA 96: 2296-3000.
Daly, T. M., Ohlemiller, K. K., Roberts, M. S., Vogler, C. A. & Sands, M. S. (2001) Gene Ther. 8, 1291-1298.
de Bont L G, Boering G, Liem R S, Eulderink F, Westesson P L (1986). Osteoarthritis and internal derangement of the temporomandibular joint: a light microscopic study. J Oral & Maxillofac Surg 44:634-43.
de Crombrugghe, B., Lefebvre, V., Behringer, R. R., Bi, W., Murakami, S., & Huang, W. (2000) Matrix Biol. 19, 389-394.
Dijkgraaf L C, de Bont L G, Boering G, Liem R S (1995). The structure, biochemistry, and metabolism of osteoarthritic cartilage: a review of the literature. J Oral Maxillofac Surg 53:1182-92.
Dijkgraaf L C. Liem R S. de Bont L G (1997). Synovial membrane involvement in osteoarthritic temporomandibular joints: a light microscopic study. Oral Surg Oral Med Oral Pathol Oral Radiol & Endodont 83:373-86.
Dijkgraaf L C. Spijkervet F K. de Bont L G (1999). Arthroscopic findings in osteoarthritic temporomandibular joints. J Oral & Maxillofac Surg 57:255-68.
Dinchuk J E, Liu R Q, Trzaskos J M (2003) COX-3: in the wrong frame in mind. Immunol Lett. 86:121.
Dreessen D, Halata Z, Strasmann T (1990). Sensory innervation of the temporomandibular joint in the mouse. Acta Anat 139:154-160.
Dubuisson D, Dennis S. The formalin test: A quantitative study of the analgesic effects of morphine, meperidine, and brainstem stimulation in rats and cats. Pain 1977; 4: 161-74.
Elbashir, S. M., J. Harborth, W. Lendeckel, A. Yalcin, K. Weberand T. Tuschl. Duplexes of 21-nucleotide RNAs mediate RNA interference in cultured mammalian cells. Nature (2001) 411:494-498.
Enlow, D. H., & Hans, M. G. (1996) in Essentials of facial growth, eds. Enlow D. H., & Hans M. G. (W.B. Saunders Co., New York).
Fernandes J C, Caron J P, Martel-Pelletier J, Javanovic D, Mineau F, Tardif G, Otterness I G, Pelletier J P (1997). Effects of tenidap on the progression of osteoarthritic lesions in a canine experimental model. Suppression of metalloprotease and interleukin-1 activity. Arthritis & Rheum 40:284-94.
Fiorucci S, Meli R, Bucci M, Cirino G (2001) Dual inhibitors of cycloxygenase and 5-lipoxygenase. A new avenue in anti-inflammatory therapy? Biochem Pharmacol 62: 1433-8.
Fitzgerald G A and Patrono C (2001). The coxibs, selective inhibitors of cyclooxygenase-2. N Engl J Med 345:433-42.
Frommer J, Monroe C W (1966). The morphology and distribution of nerve fibers and endings associated with the mandibular joint of the mouse. J Dent Res 45:1762-1766.
Fujita, T., Ohtani, J., Shigekawa, M., Kawata, T., Kaku, M., Kohno, S., Tsutsui, K., Tenjo, K., Motokawa, M., Tohma, Y., & Tanne, K. (2004) J. Dent. Res. 83, 250-254.
Furuta, Y., & Jee, W. S. (1986) Anat. Rec. 215, 305-316.
Futaki N, Arai I, Hamasaka Y, Takahashi S, Higuchi S and Otomo S (1993). Selective inhibition of NS-398 on prostanoid production in inflamed tissue in rat carrageenan-air-pouch inflammation. J Pharmacol 45:753-55.
Gillman S C, Chang J, Zeigler P R, Uhl J, Mochan E (1988). Interleukin 1 activates phospholipase A2 in human synovial cells. Arthritis Rheum 31:126-30.
Gilroy D W, Colville-Nash P R, McMaster S, Sawatzky D A, Willoughby D A, Lawrence T (2003). Inducible cyclooxygenase-derived 15-deoxy(Delta)12-14PGJ2 brings about acute inflammatory resolution in rat pleurisy by inducing neutrophil and macrophage apoptosis. FASEB 17:2269-71.
Gilroy D W, Colville-Nash P R, Willis D, Chivers J, Paul-Clark M J, Willoughby D A (1999). Inducible cyclooxygenase may have anti-inflammatory properties. Nat Med 5:698-701.
Gorlin, R. J., Cohen M. M. Jr., & Hennekam R. C. M. (1991) in Syndromes of the head and neck, eds. R. J. Gorlin, M. M. Cohen, Jr. and R. C. M. Hennekam (Oxford University Press, New York), pp. 119-177.
Gravel, R. A., Triggs-Raine, B. L. and Mahuran, D. J. (1991). Biochemistry and genetics of Tay-Sachs disease. Can. J. Neurol. Sci. 18, 419-423.
Greenwald R A, Moy W W (1997). Inhibition of collagen gelation by action of the superoxide radical. Arthritis Rheum 22:251-59.
Han R, Tsui S and Smith T J (2002). Up-regulation of prostaglandin E2 synthesis by interleukin-1β in human orbital fibroblasts involves coordinate induction of prostaglandin H synthase-2 and glutathione-dependent prostaglandin E2 synthase expression. J Biol Chem 277:163555-64.
Hannon, G. J. (2002). RNA interference. Nature 418, 244-251
Havenga M J, Vogels R, Braakman E, Kroos N, Valerio D, Hagenbeek A, van Es H H (1998). Second gene expression in bicistronic constructs using short synthetic intercistrons and viral IRES sequences. Gene. 222:319-27.
Hay, E., Lemonnier, J., Modrowski, D., Lomri, A., Lasmoles, F. and Marie, P. J. (2000). N- and E-cadherin mediate early human calvaria osteoblast differentiation promoted by bone morphogenetic protein-2. J. Cell. Physiol. 183, 117-28.
Helminen H J, Kiraly K, Pelttari A, Tammi M, Vandenberg P, Pereira R, et al. (1993). An inbred line of transgenic mice expressing an internally deleted gene for type II procollagen (COL2A1). J Clin Invest 92:582-95.
Hoffman, W. Y. and McCarthy, J. G. (1994). The effects of facial nerve ablation on craniofacial skeletal development in neonatal rabbits. Plast. Recostr. Surg. 93, 1236-1240.
Hommel, J. D., Sears, R. M., Georgescu, D., Simmons, D. L., and DiLeone, R. J. (2003). Local gene knockdown in the brain using viral-mediated RNA interference. Nat Med 9, 1539-1544.
Houston, C. S., Opitz, J. M., Spranger, J. W., Macpherson, R. I., Reed, M. H., Gilbert, E. F., Hellmann, J. and Schinzel, A. (1983). The campomelic syndrome: review, report of 17 cases, and follow-up on the currently 17-year-old boy first reported by Maroteaux et al in 1971. Am. J. Med. Genet. 15, 3-28.
Huang, J. Q., Trasler, J. M., Igdoura, S., Michaud, J., Hanal, N. and Gravel, R. A. (1997). Apoptotic cell death in mouse models of GM2 gangliosidosis and observations on human Tay Sachs and Sandhoffs diseases. Hum. Mol. Genet. 6, 1879-1885.
Huang, W., Chung, U. I., Kronenberg, H. M. and de Crombrugghe, B. (2001). The chondrogenic transcription factor Sox9 is a target of signaling by the parathyroid hormone-related peptide in the growth plate of endochondral bones. Proc. Natl. Acad. Sci. U.S.A. 98, 160-165.
Huang, W., Zhou, X., Lefebvre, V. and de Crombrugghe, B. (2000). Phosphorylation of SOX9 by cyclic AMP-dependent protein kinase A enhances SOX9's ability to transactivate a Col2a1 chondrocyte-specific enhancer. Mol. Cell. Biol. 20, 4149-4158.
Hutchins B, Patel H, Spears R (2002). Attenuation of pro-inflammatory neuropeptide levels produced by a cyclooxygenase-2 inhibitor in an animal model of chronic temporomandibular joint inflammation. J Orofac Pain 16:312-6.
Ionescu, A. M., Schwarz, E. M., Vinson, C., Puzas, J. E., Rosier, R., Reynolds, P. R. and O'Keefe, R. J. (2001). PTHrP modulates chondrocyte differentiation through AP-1 and CREB signaling. J. Biol. Chem. 276, 11639-11647.
Irwin, M. H., W. D. Pogozelski, and C A Pinkert (2002). PCR optimization for detection of transgene integration, p. 475-484. In C. A. Pinkert (ed.), Transgenic animal technology: a laboratory handbook. 2nd ed. Academic Press, Inc., San Diego.
Jakobsson P J, Thorén S, Morgenstern R, Samuelsson B (1999). Identification of human prostaglandin E synthase: a microsomal, glutathione-dependent, inducible enzyme, constituting a potential novel drug target. Proc Natl Acad Sci USA 96:7220-25.
Jantzen P T, Connor K E, DiCarlo G, Wenk G L, Wallace J L, Rojiani A M, Coppola D, Morgan D, Gordon M N (2002). Microglial Activation and β-Amyloid Deposit Reduction Caused by a Nitric Oxide-Releasing Nonsteroidal Anti-Inflammatory Drug in Amyloid Precursor Protein Plus Presenilin-1 Transgenic Mice. J Neurosci 22:2246-54.
Jiao Y, Ma X, Zhang Z (2001). Interleukin-1 increase nitric oxide synthesis through up-regulation of inducible nitric-oxide synthase by rabbit mandibular condylar cartilage cells in vitro. Chin J Stomatol 36:345-7.
Johansson A-S, Isacson G, Isberg A, et al. (1986) Distribution of substance P-like immunoreactive nerve fibers in temporomandibular joint soft tissues of monkey. Scand J Dent Res 94:225-30.
Jonakait G M, Schotland S (1990). Conditioned medium from activated splenocytes increases substance P in sympathetic ganglia. J Neurosci Res 26:24-30.
Jonakait G M, Schotland S, Hart R P (1991). Effects of lymphokines on substance P in injured ganglia of the peripheral nervous system. Ann NY Acad Sci 632:19-30.
Jonakait G M, Schotland S, Hart R P (1991). Interleukin-1 specifically increases substance P in injured sympathetic ganglia. Ann NY Acad Sci 594:222-30.
Joosten L A, Helsen M M, Saxne T, van De Loo F A, Heinegard D, van Den Berg WB (1999). IL-1 alpha beta blockade prevents cartilage and bone destruction in murine type II collagen-induced arthritis, whereas TNF-alpha blockade only ameliorates joint inflammation. J Immunol; 163:5049-55.
Jovanovic D V, Fernandes J C, Martel-Pelletier J, Jolićoeur F C, Reboul P, Laufer S, Tries S, Pelletier J P (2001). In vivo dual inhibition of cyclooxygenase and lipoxygenase by ML-3000 reduces the progression of experimental osteoarthritis: suppression of collagenase 1 and interleukin-1beta synthesis. Arthr Rheumaton 44:2320-30.
Jow R W and Clark G T (1989). Endurance and recovery from a sustained isometric contraction of human jaw elevating muscles. Arch Oral Biol 34: 857-62.
Kanaji, A., Kosuga, M., Li, X.-K., Fukuhara, Y., Tanabe, A., Kamata, Y., Azuma, N., Yamada, M., Sakamaki, T., Toyama, Y., & Okuyama, T. (2003) Mol. Ther. 8, 718-715.
Kang Y, Stein C S, Heth J A, Sinn P L, Penisten A K, Staber P D, Ratliff K L, Shen H, Barker C K, Martins I, Sharkey C M, Sanders D A, McCray P B Jr., Davidson B L (2002). In vivo gene transfer using a nonprimate lentiviral vector pseudotyped with rocs river virus glycoproteins. J Virol 76 9378-88.
Katagiri, T., Yamaguchi, A., Komaki, M., Abe, E., Takahashi, N., Ikeda, T., Rosen, V., Wozney, J. M., Fujisawa-Sehara, A., & Suda, T. (1994) J. Cell Biol. 127, 1755-1766.
Kawai Y, Kubota, Okabe E (2000). Reactive oxygen species participation in experimentally induced arthritis of the temporomandibular joint in rats. J Dent Res 79:1489-95.
Kawakami M, Okabe E (1998). Superoxide anion radical-triggered Ca+2 release from cardiac sarcoplasmic reticulum through ryanodine receptor Ca⁺² channel. Mol Pharmacol 53:497-503.
Kehl L J, Trempea T M and Hargreaves K M (2000). A new animal model for assessing mechanisms and management of muscle hyperalgesia. Pain 85:333-43.
Kido M A, Kiyoshima T, Kondo T, Ayasaka N, Moroi R, Terada Y, Tanaka T (1993). Distribution of substance P and CGRP-like immunoreactive nerve fibers in the rat temporomandibular joint. J Dent Res 72:592-598.
Kido M A, Kondo T, Ayasaka N, Terada Y, Tanaka T (1991). The peripheral distribution of trigeminal nerve fibers in the rat temporomandibular joint studied by an anterograde axonal transport method with wheat germ agglutinin-horseradish peroxidase. Arch Oral Biol 36:397-400.
Kirtikara K, S G Morham, R Raghow, S J Laulederkind, T Kanekura, S Goorhaand, L R Ballou (1998). Compensatory prostaglandin E2 biosynthesis in cyclooxygenase 1 or 2 null cells. J Exp Med 187:517-23.
Kis B, Snipes J A, Isse T, Nagy K, Busija D W. (2003) Putative cyclooxygenase-3 expression in rat brain cells. J Cereb Blood Flow Metab. 23: 1287-92.
Kitanaka J, Hashimoto H, Gotoh M, Kondo K, Sakata K, Hirasawa Y, Sawada M, Suzumura A, Marunouchi T, Matsuda T and Baba A. (1996) Expression pattern of messenger RNAs for prostanoid receptors in glial cell cultures. Brain Res 707:282-87.
Kjaer I. (1998) Crit. Rev. Oral Biol. Med. 9, 224-244.
Kjaer, I. (1990). Correlated appearance of ossification and nerve tissue in human fetal jaws. J. Craniofac. Gene. Develop. Biol. 10, 329-336.
Klineberg I (1971). Structure and function of temporomandibular joint innervation. Ann Royal Coll Surg Engl 49:268-288
Kosher, R. A., & Walker, K. H. (1983) Exp. Cell Res. 145, 145-153.
Krebsbach P H, Harrison J R, Lichtler A C, Woody C O, Rowe D W, Kream B E (1993). Transgenic expression of COL1A1-chloramphenicol acetyltransferase fusion genes in bone: differential utilization of promoter elements in vivo and in cultured cells. Mol Cell Biol 13: 5168-74.
Kuboki T, Nakanishi T, Kanyama M, Sonoyama W, Fujisawa T, Kobayashi K, et al. (1999). Direct adenovirus-mediated gene delivery to the temporomandibular joint in guinea-pigs. Arc Oral Biol 44: 701-709.
Kubota E, Kubota T, Matsumoto J, Shibata T, Murakami K l (1998). Synovial fluid cytokines and proteinases as markers of temporomandibular joint disease. J Oral Maxillofac Surg 56:192-98.
Kyrkanides S, Kambylafkas P, Miller J H, Tallents R H (2004). Non-primate lentiviral vector administration in the TMJ. J Dental Res 83: 65-70.
Kyrkanides S, Miller J H, Bowers W A, Federoff H J (2003). Transcriptional and post-translational regulation of Cre recombinase by RU486 as the basis for an enhanced inducible expression system. Mol Ther 8: 790-95.
Kyrkanides S, Miller J H, Federoff H J (2003). Systemic FIV vector administration: Transduction of CNS immune cells and Purkinje neurons. Mol Brain Res 119: 1-9.
Kyrkanides S, Moore A H, Olschowka J A, Williams J P, Hansen J T, O'Banion M K (2002). COX-2 modulates inflammation related genes in CNS radiation injury. Mol Brain Res 104: 159-69.
Kyrkanides S, O'Banion M K, Subtelny J D (2000). Non-steroidal anti-inflammatory drugs in orthodontic tooth movement: Metalloproteinase activity and collagen synthesis by endothelial cells. Am J Orthod Dentofac Orthop 118: 203-09.
Kyrkanides S, Olschowka J A, Whitley P, O'Banion M K (2001). Enhanced glial activation and expression of specific CNS inflammation-related molecules in aged versus young rats following cortical stab injury. J Neuroimmunol 119: 269-77.
Kyrkanides S, Olschowka J A, Williams J P, Hansen J T, O'Banion M K (1999). TNFα & IL-1β mediate ICAM-1 induction via microglia-astrocyte interaction in CNS radiation injury. J Neuroimmunol 95:95-106.
Kyrkanides S, Tallents R H, Macher D J, Olschowka J O, Stevens S Y (2002). Temporomandibular joint nociception: Effects of capsaicin on substance P-like immunoreactivity in the rabbit brain stem. J Orofac Pain 16: 229-235.
Kyrkanides, S., Miller, J. H., Brouxhon, S. M., Olschowka, J. A., & Federoff, H. J. (2005) Mol. Brain Res. 133, 286-298.
Lane N E (1997). Pain management in osteoarthritis: the role of COX-2 inhibitors. J Rheumatol 24 Suppl. 49:20-24.
Langenbach R, Morham S G, Tiano H F, Loftin C D, Ghanayem B I, Chulada P C, Mahler J F, Lee C A, Goulding E H, Kluckman K D, Kim H S, Smithies O (1995). Prostaglandin Synthase 1 Gene Disruption in Mice Reduces Arachidonic Acid-Induced Inflammation and Indomethacin-Induced Gastric Ulceration. Cell 83:483-92.
Lavigne P, Shi Q, Jolicoeur F C, Pelletier, Martel-Pelletier J, Fernandes J C (2002). Modulation of IL-1beta, IL-6, TNF-alpha and PGE(2) by pharmacological agents in explants of membranes from failed total hip replacement. Osteoarthritis & Cartilage 10:898-904.
Lefebvre, V., Huang, W., Harley, V. R., Goodfellow, P. N. and de Crombrugghe, B. (1997). SOX9 is a potent activator of the chondrocyte-specific enhancer of the pro alphal (II) collagen gene. Mol. Cell. Biol. 17, 2336-2346.
Lehmann, J. M., J. M. Lenhard, B. B. Oliver, G. M. Ringold and S. A. Kleiwer (1997). Peroxisome proliferator-activated receptors alpha and gamma are activated by indomethacin and other non-steroidal anti-inflammatory drugs. J Biol Chem 272:3406-10.
Li, T. F., Zuscik, M. J., Ionescu, A. M., Zhang, X., Rosier, R. N., Schwarz, E. M., Drissi, H., & O'Keefe, R. J. (2004) Exp. Cell Res. 300, 159-169.
Lipsky P E and Isakson P C (1997). Outcome of specific COX-2 inhibition in rheumatoid arthritis. J Rheumatol 24:9-14.
Lipton J A, Ship J A, Larach-Luchini S, Merskey H (1993). Estimated prevalence and distribution of reported orofacial pain in the United States. JADA 124:115-21.
Liu F, Malaval L, Aubin J E (1997). The mature osteoblasts phenotype is characterized by extensive plasticity. Exp Cell Res 232: 97-105.
Liu, Y., Wada, R., Kawai, H., Sango, K., Deng, C., Tai, T., McDonald, M. P., Araujo, K., Crawley, J. N., Bierfreund, U., Sandhoff, K., Suzuki, K. and Proia, R. L. (1999). A genetic model of substrate deprivation therapy for a glycosphingolipid storage disorder. J. Clin. Invest. 103, 497-505.
Macher D J, Westesson P L, Brooks S L Hicks D and Tallents R H (1992). Temporomandibular joint surgically created disc displacement causes arthrosis in the rabbit. Oral Surg Oral Med Oral Pathol 73:645-49.
Maguire-Zeiss K A, Bowers W J, Federoff H J (2002). Somatic mosaic approaches and the aging brain. Neurobiol Aging 23:977-84.
Malmberg A B and Yaksh T L (1995). Cyclooxygenase inhibition and the spinal release of prostaglandin E2 and amino acids evoked by paw formalin injection: a microdialysis study in unanesthetized rats. J Neurosci 15:2768-76.
Mancini J A, Blood K, Guay J, Gordon R, Claveau D, Chan C C and Riendeau R (2001). Cloning, expression, and up-regulation of inducible rat prostaglandin e synthase during lipopolysaccharide-induced pyresis and adjuvant-induced arthritis. J Biol Chem 276:4469-75.
Mankin, K. P., & Zaleske, D. J. (1998) J. Pediater. Orthoped. 18, 145-148.
Marie, P. J. (2002). Role of N-cadherin in bone formation. J. Cell. Physiol. 190, 297-305.
Martel-Pelletier J, Pelletier J P, Fahmi H (2003). Cyclooxygenase-2 and prostaglandins in articular tissues. Semin Arthritis Rheum 33:155-67.
Martin W R, Eades C G (1961). Demonstration of tolerance and physical dependence in the dog following a short-term infusion of morphine. J Pharmacol Exp Ther 133: 262-270.
Masaki M, Matsushita M and Wakitani K. Inhibitory effects of JTE-522, a novel prostaglandin H synthase-2 inhibitor, on adjuvant-induced arthritis and bone changes in rats. Inflamm Res 47:187-92.
Matsubara T, Ziff M (1986). Increased superoxide anion release from human endothelial cells in response to cytokines. J Immunol 137:3295-98.
McCord J M (1974). Free radicals and inflammation: protection of synovial fluid by superoxide dismutase. Science 185:529-31.
Miyamoto, M., Ito, H., Mukai, S., Kobayashi, T., Yamamoto, H., Kobayashi, M., Maruyama, T., Akiyama, H. and Nakamura, T. (2003). Simultaneous stimulation of EP2 and EP4 is essential to the effect of prostaglandin E2 in chondrocyte differentiation. Osteoarthr. Cart. 11, 644-652.
Mizukawa H, Okabe E (1997). Inhibition by singlet molecular oxygen of the vascular reactivity in rabbit mesenteric artery. Br J Pharmacol 121:63-70.
Molin C (1972). Vertical isometric muscle forces of the mandible. A comparative study of subjects with and without manifest mandibular pain dysfunction syndrome. Acta Odontol Scand 30:485-99.
Moore A H, Olschowka J A, O'Banion M K (2004). Intraparenchymal administration of interleukin-1beta induces cyclooxygenase-2-mediated expression of membrane- and cytosolic-associated prostaglandin E synthases in mouse brain. J Neuroimmunol 148: 32-40.
Moos V, Fickert S, Muller B, Weber U, Sieper J (1999). Immunohistological analysis of cytokine expression in human osteoarthritic and healthy cartilage. J Rheumatol 26:870-9.
Morham S G, Langenbach R, Loftin C D, Tiano H F, Vouloumanos N, Jennette J C, Mahler J F, Kluckman K D, Ledford A, Lee C A, Smithies O (1995). Prostaglandin Synthase 2 Gene Disruption Causes Severe Renal Pathology in the Mouse. Cell 83:472-82.
Mueller-Decker K, Hirschner W, Marks F, Fuerstenberger G (2002). The Effects of Cyclooxygenase Isozyme Inhibition on Incisional Wound Healing in Mouse Skin. J Invest Dermatol 119: 1189-95.
Munier-Lehmann, H, Mauxion, F., Bauer, U., Lobel, P., & Hoflack, B. (1996) J. Biol. Chem. 271, 15166-15174.
Murakami M, Naraba H, Tanioka T, Semmyo N, Nakatani Y, Kojima F, Ikeda T, Fueki M, Ueno A, Oh-ishi S and Kudo I (2000). Regulation of prostaglandin E2 biosynthesis by inducible membrane-associated prostaglandin E2 synthase that acts in concert with cyclooxygenase-2. J Biol Chem 275:32783-92.
Murray R and Spiegel S (1988). Theory and Problems of Statistics, 2nd ed. Schaum's Outline Series, McGraw-Hill Publishing, New York, Chapter 5, p 110-21.
Myers S L, Flusser D, Brandt K D, Heck D A (1992). Prevalence of cartilage shards in synovium and their association with synovitis in patients with early and endstage osteoarthritis. J Rheumatol 19:1247-51.
Nagy A, Gertsenstein M, Vintersten K, and Behringer R. (2003) Manipulating the Mouse Embryo: A Laboratory Manual. 3rd ed., Cold Spring Harbor Laboratory Press, Cold Spring Harbor, N.Y.
Narumiya A and FitzGerald G A (2001). Genetic and pharmacological analysis of prostanoid receptor function. J Clin Invest 108:25-30.
Narumiya S, Sugimoto Y, Ushikuni F (1999). Prostanoid receptors: structures, properties and functions. Physiol. Rev. 79:1193-1226.
Ngo L, Jay G (2002). Analysis of transgene expression, p. 486-513. In C A Pinkert (ed.), Transgenic animal technology: a laboratory handbook. 2nd ed. Academic Press, Inc., San Diego.
Norisue M, Todoki K, Okabe E (1997). Inhibition by hydroxyl radicals of calcitonin gene-related peptide-mediated neurogenic vasorelaxation in isolated canine lingual artery. J Pharmacol Exp Ther 280:492-500.
Norsdenskiold U M and Grimby G (1993). Grip force in patients with rheumatoid arthritis and fibromyalgia and in healthy subjects: a study with the Grippit instrument. Scand J Rheumatol 22: 14-9.
O'Keefe, R. J., Crabb, I., Puzas, J. E., & Rosier, R. N. (1992) J. Bone Miner. Res. 7, 397-404.
O'Banion M K (1999). Cyclooxygenase-2: molecular biology, pharmacology, and neurobiology. Crit Rev Neurobiol 13: 45-82.
O'Banion M K, Sadowski H B, Winn V, Young D A (1991). A serum- and glucocorticoid-regulated 4-kilobase mRNA encodes a cyclooxygenase-related protein. J Biol Chem 266: 23261-7.
O'Banion M K, Winn V D, Young D A (1992). cDNA cloning and functional activity of a glucocorticoid-regulated inflammatory cyclooxygenase. Proc Natl Acad Sci U.S.A. 89:4888-92.
O'Byrne E M, Blancuzzi V J, Wilson D E, et al. (1990). Increased intra-articular substance P and prostaglandin E2 following injection of interleukin-1 in rabbits. Int J Tissue React 12:11-4.
Ochi T, Ohkubo Y, Mutoh S (2003). Role of cyclooxygenase-2, but not cyclooxygenase-1, on type II collagen-induced arthritis in DBA/1J mice. Biochem Pharmacol 66:1055-60.
Ogura N, Tobe M, Sakamaki H, Kujiraoka H, Akiba M, Abiko Y, Nagura H (2002). Interleukin-1 beta induces interleukin-6 mRNA expression and protein production in synovial cells from human temporomandibular joint. J Oral Pathol Med 31:353-60.
Overbeek P A (2002). DNA microinjection and transgenic animal production, p. 72-112. In C. A. Pinkert (Ed.), Transgenic animal technology: a laboratory handbook. 2nd ed. Academic Press, Inc., San Diego.
Paddison, P. J., Caudy, A. A., Sachidanandam, R., and Hannon, G. J. (2004). Short hairpin activated gene silencing in mammalian cells. Methods Mol Biol 265, 85-100.
Paddison, P. J., A. A. Caudyand G. J. Hannon. Stable suppression of gene expression by RNAi in mammalian cells. Proc. Natl. Acad. Sci. USA (2002) 99:1443-1448.
Pazzaglia, U. E., Beluffi, G., Castello, A., Coci, A., & Zatti, G. (1992) Clin. Orthopaed. Rel. Res. 276, 283-290.
Peel A L. Zolotukhin S. Schrimsher G W. Muzyczka N. Reier P J. Efficient transduction of green fluorescent protein in spinal cord neurons using adeno-associated virus vectors containing cell type-specific promoters. [Journal Article] Gene Therapy. 4(1):16-24, 1997
Pfeifer A, Brandon E P, Kootstra Neeltje, Gage F H, Verma I M (2001). Delivery of the Cre recombinase by a self-deleting lentiviral vector: Efficient gene targeting in vivo. Proc Natl Acad Sci U.S.A. 98: 11450-5.
Phaneuf, D., Wakamatsu, N., Huang, J.-Q., Borowski, A., Peterson, A. C., Fortunato, S. R., Ritter, G., Igdoura, S. A., Morales, C. R., Benoit, G., Akerman, B. R., Leclerc, D., Hanai, N., Marth, J. D., Trasler, J. M. and Gravel, R. A. (1996). Dramatically different phenotypes in mouse models of human Tay-Sachs and Sandhoff diseases. Hum. Mol. Genet. 5, 1-14.
Pinkert C. A. (2002) Transgenic Animal Technology: A Laboratory Handbook 2nd ed., Academic Press, San Diego.
Pinkert C A (2003). Transgenic animal technology: Alternatives in genotyping and phenotyping. Comp Med 53:116-29.
Poeschla E M, Wong-Stall F, Looney D l (1998). Efficient transduction of nondividing human cells by feline immunodeficiency virus lentiviral vectors. Nature Med 4: 354-357.
Pohl M, Braz J (2001). Gene therapy of pain: emerging strategies and future directions. Eur J Pharmacol 429: 39-48.
Polites H G, Pinkert C A (2002). DNA microinjection and transgenic animal production, p. 15-70. In C. A. Pinkert (ed.), Transgenic animal technology: a laboratory handbook. 2nd ed. Academic Press, Inc., San Diego.
Portanova J P, Zhang Y, Anderson G D, Hauser S D, Masferrer J L, Seibert K, Gregory S A, Isakson P C. (1996) Selective neutralization of prostaglandin E2 blocks inflammation, hyperalgesia, and interleukin 6 production in vivo. J. Exp. Med 184:883-91.
Purpura, D. P. and Suzuki, K. (1976). Distortion of neuronal geometry and formation of aberrant synapses in neuronal storage disease. Brain Res. 116, 1-21.
Ratcliffe A, Billingham M E, Saed-Nejad F, Muir H, Hardingham T E (1992). Increased release of matrix components from articular cartilage in experimental canine osteoarthritis. J Ortho Res 10:350-8.
Revell P A, Mayston V, Lalor P, Mapp P (1988). The synovial membrane in osteoarthritis: a histological study including the characterisation of the cellular infiltrate present in inflammatory osteoarthritis using monoclonal antibodies. Ann Rheum Dis 47:300-7.
Ricote M, Li A C, Willson T M, Kelly C J and Glass C K (1998). The peroxisome proliferator-activated receptor-□ is a negative regulator of macrophage activation. Nature 391:79-82.
Ritchlin C T, Haas-Smith S A, Li P, Hicks D G, Schwarz E M (2003). Mechanisms of TNF\tilde{□} and RANKL-mediated osteoclastogenesis and bone resorption in psoriatic arthritis. J Clin Investig 111:821-31.
Roberts C R, Roughley P J, Mort J S (1989). Degradation of human proteoglycan aggregate induced by hydrogen peroxide. Protein fragmentation, amino acid modification and hyaluronic acid cleavage. Biochem J 259:805-11.
Rockman, H. A. (1997). Uncoupling of G-protein coupled receptors in vivo: insights from transgenic mice. Adv. Exp. Med. Biol. 430, 67-72.
Romfh J H, Capra N F, Gatipon G B (1979). Trigeminal nerve and temporomandibular joint of the cat: A horseradish peroxidase study. Exp Neurol 65: 99-106.
Rosenberg, A. (1999). Bones, joints and soft tissue tumors. In Robbins Pathologic Basis of Disease, 6th edition (ed. Cotran, Kumar & Collins), W. B. Saunders Co, New York.
Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirus-based system to functionally silence genes in primary mammalian cells, stem cells and transgenic mice by RNA interference. Nat Genet 33, 401-406.
Salvemini D, Misko T P, Masferrer J L (1993). Nitric oxide activates cycloxygenase enzymes. Proc Natl Acad Sci 90:7240-44.
Sango, K., McDonald, M. P., Crawley, J. N., Mack, M. L., Tifft, C. J., Skop, E., Starr, C. M., Hoffmann, A., Sandhoff, K., Suzuki, K. and Proia, R. L. (1996). Mice lacking both subunits of lysosomal β-hexosaminidase display gangliosidosis and mucopolysaccharidosis. Nature Genet. 14, 348-352.
Sango, K., Yamanaka, S., Hoffman, A., Okuda, Y., Grinberg, A., Westphal, H., McDonald, M. P., Crawley, J. N., Sandhoff, K., Suzuki, K. and Proia, R. L. (1995). Mouse models of Tay-Sachs and Sandhoff diseases differ in neurologic phenotype and ganglioside metabolism. Nature Genet. 11, 170-176.
Schwarz E M, Looney R J, O'Keefe R J (2000). Anti-TNF-alpha therapy as a clinical intervention for periprosthetic osteolysis. Arthr Res 2:165-8.
Scott-Burden T, Bosley J P, Rosenstrauch D, Henderson K D, Clubb F J, Eichstaedt H C, Eya K, Gregoric I, Myers T J, Radovancevic B, Frazier O H (2002). Use of autologous auricular chondrocytes for lining artificial surfaces: A feasibility study. Ann Thorac Surg 73: 1528-33.
Sessle B J, Hu J W (1991). Mechanisms of pain arising from articular tissues. Can J Physiol Pharmacol 69: 617 626.
Shaftel S S, Olschowka J A, Hurley S D, Moore A H, O'Banion M K (2003). COX-3: a splice variant of cyclooxygenase-1 in mouse neural tissue and cells. Brain Res Mol Brain Res. 119: 213-5.
Shin S-j, Fermor B, Weinberg J B, Pisetsky D S, Guilak F (2003). Regulation of matrix turnover in meniscal explants: role of mechanical stress, interleukin-1, and nitric oxide. J Appl Physiol.; 95:308-13.
Silveri, C. P., Kaplan, F. S., Fallon, M. D., Bayever, E., & August, C. S. (1991) Clin. Orthopaed. Rel. Res. 269, 305-311.
Sinsel, N. K., Opdebeeck, H., Guelinckx, P. J. (1998). The effect of unilateral partial facial paralysis and muscle ablation on craniofacial growth and development: An experimental study in the rabbit. Plast. Reconstr. Surg. 102, 1894-1912.
Siqueira-Junior J M, Peters R R, Brum-Fernandes A J, Ribeiro-do-Valle R M (2003). Effects of valeryl salicylate, a COX-1 inhibitor, on models of acute inflammation in mice. Pharmacol Res 48:437-43.
Smith M D, Triantafillou S, Parker A, Youssef P P, Coleman M (1997). Synovial membrane inflammation and cytokine production in patients with early osteoarthritis. J Rheumatol 24:365-71.
Smith, W L, DeWitt D L, Garavito R M (2000). Cyclooxygenases: structural, cellular, and molecular biology. Annu Rev Biochem 69:145-82.
Srinivas S, Watanabe T, Lin C-S, Williams C, Tanabe Y, Jessell T, Costaniti F (2002). Cre reporter strains produced by targeted insertion of EYFP and ECFP into the ROSA26 locus. BMC Develop Biol 1:4-11.
Stegenga B, de Bont L G, Boering G (1989). Osteoarthrosis as the cause of craniomandibular pain and dysfunction: a unifying concept. J Oral Maxillofacial Surg 47:249-56.
Stegenga B, de Bont L G, Boering G, Van Willigen J D (1991). Tissue responses to degenerative changes in the temporomandibular joint: a review. J Oral Maxillofacial Surg 49:1079-88.
Sternberg N, Hamilton D (1981). Bacteriophage P1 site-specific recombination. Recombination between loxP sites. J Mol Biol 150: 467-86.
Stichtenoth D O, Thoren S, Bian H, Peters-Golden M, Jakobsson P J and Crofford L (2001). Microsomal prostaglandin E synthase is regulated by proinflammatory cytokines in primary rheumatoid synovial cells. J. Immunol. (2001) 167:469-74.
Sugimoto Y, Narumiya S and Ichikawa A (2000). Distribution and function of prostanoid receptors: studies from knockout mice. Prog Lipid Res 39:289-314.
Surnii H, Inoue H, Onoue J, Mori A, Oda T, Tsubokura T (1996). Superoxide dismutase activity in arthropathy: its role and measurement in the joints. Hiroshima J Med Sci 45:51-55.
Suzuki T, Segami N, Nisimura M, Nojima T (2002). Co-expression of interleukin-1beta and tumor necrosis factor alpha in synovial tissues and synovial fluids of temporomandibular joint with internal derangement: comparison with histological grading of synovial inflammation. J of Oral Pathology Med 31:549-57.
Suzuki, K., Sango K., Proia R. L., & Langman C. (1997) J. Neuropath. Exp. Neurol. 56, 693-703.
Takeda, S., Elefteriou, F., Levasseur, R., Liu, X., Zhao, L., Parker, K. L., Armstrong, D., Ducy, P. and Karsenty G. (2002). Leptin regulates bone formation via the sympathetic nervous system. Cell 111, 305-317.
Tallents R H, Macher D J, Rivoli P, Scapino R, Puzas J E, Katzberg R W (1990). An animal model for meniscus displacement in the rabbit. J Craniomandib Disord Facial Oral Pain 4:233-40.
Tanaka A, Hase S, Miyazawa T, Ohno R, Takeuchi K (2002). Role of cyclooxygenase (COX)-1 and COX-2 inhibition in nonsteroidal anti-inflammatory drug-induced intestinal damage in rats: relation to various pathogenic events. J Pharmacol Exp Ther 303: 1248-54.
Taniike, M., Yamanaka, S., Proia, R. L., Langaman, C., Bone-Turrentine, T. and Suzuki, K. (1995). Neuropathology of mice with targeted disruption of Hexa gene, a model of Tay-Sachs disease. Acta Neuropathol. 89, 296-304.
Tanioka T, Nakatani Y, Semmyo N, Murakami M and Kudo I (2000). Molecular identification of cytosolic prostaglandin E2 synthase that is functionally coupled with cylooxygenase-1 in immediate prostaglandin E2 biosynthesis. J Biol Chem 275:32775-82.
Tawara T, Shingu M, Nobunaga M, Naono T (1991). Effects of recombinant human IL-Iβ on production of prostaglandin E2, leukotriene B4, NAG, and superoxide by human synovial cells and chondrocytes. Inflammation 15:145-57.
Thilander B (1964). Innervation of the temporomandibular disc in Man. Acta Odont Scan 22:151-156.
Tiku M L, Liesch J B, Robertson F M (1990). Production of hydrogen peroxide by rabbit articular chondrocytes. Enhancement by cytokines. J Immunol 145:690-96.
Tilley S L, Coffman T M and Koller B H (2001). Mixed messages: modulation of inflammation and immune response by prostaglandins and thromboxanes. J Clin Invest 108:15-23
Tinkle B T, Jay G (2002). Analysis of transgene integration, p. 459-474. In C A Pinkert (ed.), Transgenic animal technology: a laboratory handbook. 2nd ed. Academic Press, Inc., San Diego.
Tiscornia, G., Singer, 0., Ikawa, M., and Verma, I. M. (2003). A general method for gene knockdown in mice by using lentiviral vectors expressing small interfering RNA. Proc Natl Acad Sci USA 100, 1844-1848.
Tuschl, T. RNA interference and small interfering RNAs. Chembiochem (2001) 2:239-245.
Ueno, K., Haba, T., Woodbury, D., Price, P., Anderson, R., & Jee, W. S. (1985) Bone 6, 79-86.
Vane J R, Bakhle Y S, Botting R M (1998). Cyclooxygenases 1 and 2. Annu. Rev. Pharmocol. Toxicol. 38:97-120.
Vignon, E., Broquet, P., Mathieu, P., Louisot, P. and Richard, M. (1990. Histaminergic H1, serotoninergic, beta adrenergic and dopaminergic receptors in human osteoarthritic cartilage. Biochem. Intl. 20, 251-255
von Specht, B. U., Geiger, B., Arnon, R., Passwell, J., Keren, G., Goldman, B., & Padeh, B. (1979) Neurol. 29, 848-854.
Wada S, Okabe E (1997). Susceptibility of caffeine and INS(1,4,5)P3 induced contractions to oxidants in permeabilized vascular smooth muscle. Eur J Pharmacol 320:51-59.
Wagner, T., Wirth, J., Meyer, J., Zabel, B., Held, M., Zimmer, J., Pasantes, J., Bricarelli, F. D., Keutel, J. and Hustert, E. (1994). Autosomal sex reversal and campomelic dysplasia are caused by mutations in and around the SRY-related gene SOX9. Cell 79, 1111-1120.
Walkley, S. U., Baker, H. J., Rattazzi, M. C., Haskins, M. E. and Wu, J. Y. (1991). Neuroaxonal dystrophy in neuronal storage disorders: evidence for major GABAergic neuron involvement. J. Neurol. Sci. 104, 1-8.
Wang J B, Imai Y, Eppler C M, Gregor P, Spivak C E, Uhl G R. (1993). mu opiate receptor: cDNA cloning and expression. PNAS USA 90:10230-4
Watanabe, H., Yamada, Y. (2002). Chondrodysplasia of gene knockout mice for aggrecan and link protein. Glycoconjug. J. 19, 269-273.
Webb G R. Westacott C I. Elson C J (1998). Osteoarthritic synovial fluid and synovium supernatants up-regulate tumor necrosis factor receptors on human articular chondrocytes. Osteoarthr & Cartil 6167-76.
Wilhelmi G, Faust R (1976). Suitability of the C57 black mouse as an experimental animal for the study of skeletal changes due to ageing, with special reference to osteo-arthrosis and its response to tribenoside. Pharmacol 14:289-96.
Wingren A G, Bjorkdahl O, Labuda T, Bjork L, Andersson U, Gullberg U, Hedlund G, Sjogren H O, Kalland T, Widegren B, Dohlsten M (1996). Fusion of a signal sequence to the interleukin-1 beta gene directs the protein from cytoplasmic accumulation to extracellular release. Cell Immunol 169:226-37.
Wink C S, St'Onge M, Zimny M L (1992). Neural elements in the human temporomandibular articular disc. J Oral Maxillofac Surg 50:334-337
Wong, P. Y., Majeska, R. J., & Wuthier, R. E. (1977) Prostagland. 14, 839-851.
Woodruff T, Blake D R, Freeman J, Andrews F J, Salt P, Lunec J (1986). Is chronic synovitis an example of reperfusion injury? Ann Rheum Dis 45:608-11.
Wu C L, Garry M G, Zollo R A, Yang J (2001). Gene therapy for the management of pain: part II: molecular targets. Anesthesiology 95: 216-240.
Yaksh T L, Dirig D M, Conway C M, Svensson C, Luo Z D, Isakson P C (2001). The acute antihyperalgesic action of nonsteroidal, anti-inflammatory drugs and release of spinal prostaglandin E2 is mediated by the inhibition of constitutive spinal cyclooxygenase-2 (COX-2) but not COX-1. J Neurosci 21:5847-53.
Yamamoto Y, Yin M J, Lin K M and Gaynor R B (1999). Sulindac inhibits activation of the NF-κB pathway. J Biol Chem 274:27307-314.
Yamanaka, S., Johnson, M. D., Grinberg, A., Westphal, H., Crawley, J. N., Taniike, M., Suzuki, K. and Proia, R. L. (1994). Targeted disruption of the Hexa gene results in mice with biochemical and pathologic features of Tay-Sachs disease. Proc. Natl. Acad. Sci. U.S.A. 91, 9975-9979.
Yang, D., F. Buchholz, Z. Huang, A. Goga, C.-Y. Chen, F. M. Brodskyand J. M. Bishop. Short RNA duplexes produced by hydrolysis with Escherichia coli RNase III mediate effective RNA interference in mammalian cells. Proceedings of the National Academy of Sciences (2002) 99:9942-994.
Yin M J, Yamamoto Y and Gaynor R B (1998). The anti-inflammatory agents aspirin and salicylate inhibit the activity of IkB kinase-β. Nature 396:77-80.
Yoshida H, Fukumura Y, Fujita S, Nishida M, Iizuka T (2002). The expression of cyclooxygenase-2 in human temporomandibular joint samples: an immunohistochemical study. J Oral Rehab 29:1146-52.
Yoshino K, Kawagishi S, Amano N (1998). Morphological characteristics of primary sensory and post-synaptic sympathetic neurons supplying the temporomandibular joint in the cat. Arc Oral Biol; 43: 679-686.
Zhang J, S Goorha, R Raghowand, L R Ballou (2002). The tissue-specific, compensatory expression of cyclooxygenase-1 and -2 in transgenic mice. Prostagland Other Lipid Mediat 67:121-35.
Zhang, X., Ziran, N., Goate,r J. J., Schwarz, E. M., Puzas, J. E., Rosier, R. N., Zuscik, M., Drissi, H. and O'Keefe, R. J. (2004). Primary murine limb bud mesenchymal cells in long-term culture complete chondrocyte differentiation: TGF-beta delays hypertrophy and PGE2 inhibits terminal differentiation. Bone 34, 809-817.
Zhu J, Musco M L, Grace M J (1999). Three-color flow cytometry analysis of tricistronic expression of eBFP, eGFP, and eYFP using EMCV-IRES linkages. Cytometry 37: 51-9.
Zhu X, Conklin D, Eisenach J C (2003). Cyclooxygenase-1 in the spinal cord plays an important role in postoperative pain. Pain 104:15-23.
Zuscik, M. J., D'Souza, M., Ionescu, A. M., Gunter, K. K., Gunter, T. E., O'Keefe, R. J., Schwarz, E. M., Puzas, J. E. and Rosier R. N. (2002). Growth plate chondrocyte maturation is regulated by basal intracellular calcium. Exp. Cell Res. 276, 310-319.
Zuscik, M. J., Puzas, J. E., Rosier, R. N., Gunter, K. K. and Gunter, T. E. (1994). Cyclic-AMP-dependent protein kinase activity is not required by parathyroid hormone to stimulate phosphoinositide signaling in chondrocytes but is required to transduce the hormone's proliferative effect. Arch. Biochem. Biophys. 315, 352-361.

What is claimed is:

1. A method for treating or preventing peripheral inflammation in a subject, comprising administering an inflammation inhibitor to the central nervous system of the subject.

2. The method of claim 1, wherein the subject has osteoarthritis, rheumatoid arthritis, gout, ankylosing spondylitis, juvenile arthritis, systemic lupus erythematosus (lupus), scleroderma, or fibromyalgia.

3. A method for treating or preventing bone disease in a subject, comprising administering an inflammation inhibitor to the central nervous system of the subject.

4. A method for treating or preventing chronic pain in a subject, comprising administering an inflammation inhibitor to the central nervous system of the subject.

5. The method of claim 1, wherein the inflammatory mediator is administered directly to the dorsal hom, cisterna magna, or thecal sac.

6. A method for treating or preventing a brain disorder in a subject, comprising administering an inflammation inhibitor to a site of peripheral inflammation in the subject.

7. The method of claim 6, wherein the subject has Alzheimer's disease.

8. The method of claim 6, wherein the subject has osteoarthritis, rheumatoid arthritis, gout, ankylosing spondylitis, juvenile arthritis, systemic lupus erythematosus (lupus), scleroderma, or fibromyalgia.

9. The method of claim 1, wherein the inflammation inhibitor is a viral vector, wherein delivery of the vector to a cell inhibits a mediator of inflammation.

10. The method of claim 9, wherein the vector comprises a nucleic acid operably linked to an expression control sequence and wherein the nucleic acid inhibits expression of the mediator of inflammation.

11. The method of claim 10, wherein the nucleic acid is an siRNA.

12. The method of claim 11, wherein the siRNA inhibits gene expression of COX-1.

13. The method of claim 12, wherein the siRNA comprises the nucleic acid sequence SEQ ID NO:49.

14. The method of claim 11, wherein the siRNA inhibits gene expression of COX-2.

15. The method of claim 14, wherein the siRNA comprises the nucleic acid sequence SEQ ID NO: 53.

16. The method of claim 11, wherein the siRNA inhibits gene expression of mPGES.

17. The method of claim 16, wherein the siRNA comprises the nucleic acid sequence SEQ ID NO: 59.

18. The method of claim 11, wherein the siRNA inhibits gene expression of cPGES.

19. The method of claim 18, wherein the siRNA comprises the nucleic acid sequence SEQ ID NO: 43.

20. The method of claim 29, wherein the vector comprises a nucleic acid that encodes a polypeptide that inhibits the binding of the mediator of inflammation to its receptor.

21. The method of claim 20, wherein the polypeptide inhibits the binding of IL-1β to an IL-1 receptor.

22. The method of claim 21, wherein the polypeptide is IL-1ra.

23. The method of claim 22, wherein the polypeptide is human IL-1ra.

24. The method of claim 23, wherein the nucleic acid comprises the sequence set forth in SEQ ID NO:5.

25. The method of claim 23, wherein the nucleic acid encodes a polypeptide with at least 70%, 75%, 80%, 85%, 90%, 95% identity to the sequence set forth in SEQ ID NO:38.

26. The The method of claim 25, wherein the any change is a conservative change

27. The method of claim 23, wherein the nucleic acid hybridizes to SEQ ID NO:5 under stringent conditions.

28. The method of claim 10, wherein the expression control sequence is a constitutive promoter.

29. The method of claim 28, wherein the promoter is a CMV promoter.

30. The method of claim 29, wherein the CMV promoter comprises the nucleic acid sequence set forth in SEQ ID NO:15.

31. The method of claim 28, wherein the promoter is a beta actin promoter.

32. The method of claim 31, wherein the the beta actin promoter comprises the nucleic acid sequence set forth in SEQ ID NO: 16.

33. The method of claim 10, wherein the expression control sequence is a tissue specific promoter.

34. The method of claim 10, wherein the expression control sequence is an inducible promoter.

35. The method of claim 10, wherein the vector further comprises a marker sequence.

36. The method of claim 10, wherein the vector comprises a lentivirus.

37. The method of claim 36, wherein the vector comprises a feline immunodeficiency virus.

38. The method of claim 36, wherein the vector comprises a human immunodeficiency virus.

39. The method of claim 1, further comprising administering an opioid receptor or a nucleic acid encoding an opioid receptor to a site of peripheral inflammation in the subject.

40. The method of claim 39, wherein the opioid receptor is a μ-opioid receptor.

41. The method of claim 40, wherein the μ-opioid receptor has a nucleic sequence with at least 80% identity to the sequence set forth in SEQ ID NO:92.


SEQUENCE LISTING (148 sequences)

SEQ ID NO: 1 - DNA, 2943 bp, Artificial Sequence
  Description of Artificial Sequence  note  = synthetic construct
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 2 - DNA, 1498 bp, Artificial Sequence
  Description of Artificial Sequence  note  = synthetic construct
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 3 - DNA, 464 bp, Artificial Sequence
  Description of Artificial Sequence  note  = synthetic construct
gcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaag

SEQ ID NO: 4 - DNA, 539 bp, Artificial Sequence
  Description of Artificial Sequence  note  = synthetic construct
atggaaatctgcagaggcctccgcagtcacctaatcactctcctcctcttcctgttccattcagagacgatctgcgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaag

SEQ ID NO: 5 - DNA, 1760 bp, Artificial Sequence
  Description of Artificial Sequence  note  = synthetic construct
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaa

SEQ ID NO: 6 - DNA, 534 bp, Artificial Sequence
  Description of Artificial Sequence  note  = synthetic construct
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 7 - DNA, 534 bp, Artificial Sequence; identical to SEQ ID NO: 6
  Description of Artificial Sequence  note  = synthetic construct

SEQ ID NO: 8 - DNA, 4849 bp, Artificial Sequence
  Description of Artificial Sequence  note  = synthetic construct
tagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgtttttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctcgacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaa

SEQ ID NO: 9 - DNA, 1436 bp, Artificial Sequence
  Description of Artificial Sequence  note  = synthetic construct
gggatgggagatactgttgtggtcacctctggaaaatacattctgctactcttaaaaactagtgacgctcatacaaatcaacagaaagagcttctgaaggaagactttaaagctgcttctgccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 10 - DNA, 2554 bp, Artificial Sequence
  Description of Artificial Sequence  note  = synthetic construct
gcgccatgagccggagtctcttgctccggttcttgctgttcctgctcctgctcccgccgctccccgtcctgctcgcggacccaggggcgcccacgccagtgaatccctgttgttactatccatgccagcaccagggcatctgtgtccgcttcggccttgaccgctaccagtgtgactgcacccgcacgggctattccggccccaactgcaccatccctggcctgtggacctggctccggaattcactgcggcccagcccctctttcacccacttcctgctcactcacgggcgctggttctgggagtttgtcaatgccaccttcatccgagagatgctcatgcgcctggtactcacagtgcgctccaaccttatccccagtccccccacctacaactcagcacatgactacatcagctgggagtctttctccaacgtgagctattacactcgtattctgccctctgtgcctaaagattgccccacacccatgggaaccaaagggaagaagcagttgccagatgcccagctcctggcccgccgcttcctgctcaggaggaagttcatacctgacccccaaggcaccaacctcatgtttgccttctttgcacaacacttcacccaccagttcttcaaaacttctggcaagatgggtcctggcttcaccaaggccttgggccatggggtagacctcggccacatttatggagacaatctggagcgtcagtatcaactgcggctctttaaggatgggaaactcaagtaccaggtgctggatggagaaatgtacccgccctcggtagaagaggcgcctgtgttgatgcactacccccgaggcatcccgccccagagccagatggctgtgggccaggaggtgtttgggctgcttcctgggctcatgctgtatgccacgctctggctacgtgagcacaaccgtgtgtgtgacctgctgaaggctgagcaccccacctggggcgatgagcagcttttccagacgacccgcctcatcctcataggggagaccatcaagattgtcatcgaggagtacgtgcagcagctgagtggctatttcctgcagctgaaatttgacccagagctgctgttcggtgtccagttccaataccgcaaccgcattgccatggagttcaaccatctctaccactggcaccccctcatgcctgactccttcaaggtgggctcccaggagtacagctacgagcagttcttgttcaacacctccatgttggtggactatggggttgaggccctggtggatgccttctctcgccagattgctggccggatcggtgggggcaggaacatggaccaccacatcctgcatgtggctgtggatgtcatcagggagtctcgggagatgcggctgcagcccttcaatgagtaccgcaagaggtttggcatgaaaccctacacctccttccaggagctcgtaggagagaaggagatggcagcagagttggaggaattgtatggagacattgatgcgttggagttctaccctggactgcttcttgaaaagtgccatccaaactctatctttggggagagtatgatagagattggggctcccttttccctcaagggtctcctagggaatcccatctgttctccggagtactggaagccgagcacatttggcggcgaggtgggctttaacattgtcaagacggccacactgaagaagctggtctgcctcaacaccaagacctgtccctacgtttccttccgtgtgccggatgccagtcaggatgatgggcctgctgtggagcgaccatccacagagctctgaggggcaggaaagcagcattctggaggggagagctttgtgcttgtcattccagagtgctgaggccagggctgatggtcttaaatgctcattttctggtttggcatggtgagtgttggggttgacatttagaactttaagtctcacccattatctggaatattgtgattctgtttattcttccagaatgctgaactccttgttagcccttcagattgttaggagtggttctcatttggtctgccagaatactgggttcttagttgacaacctagaatgtcagatttctggttgatttgtaacacagtcattctaggatgtggagctactgatgaaatctgctagaaagttagggggttcttattttgcattccagaatcttgactttctgattggtgattcaaagtgttgtgttccctggctgatgatccagaacagtggctcgtatcccaaatctgtcagcatctggctgtctagaatgtggatttgattcattttcctgttcagtgagatatcatagagacggagatcctaaggtccaacaagaatgcattccctgaatctgtgcctgcactgagagggcaaggaagtggggtgttcttcttgggacccccactaagaccctggtctgaggatgtagagagaacaggtgggctgtattcacgccattggttggaagctaccagagctctatccccatccaggtcttgactcatggcagctgtttctcatgaagctaataaaattcgccc

SEQ ID NO: 11 - DNA, 4465 bp, Artificial Sequence
  Description of Artificial Sequence  note  = synthetic construct
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 12 - DNA, 1805 bp, Artificial Sequence
  Description of Artificial Sequence  note  = synthetic construct
gctgctcctctgtcgagctgatcacacccacagttgagctgcgctggccagagatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaccagcagctgatgcctccttggccaccagaccatgggccaagagccgccgtggctatacctggggacttgatgttccttccagattgtggtgggccctgagtcctggtttcctggcagcctgctgcgcgtgtgggtctctgggcacagtgggcctgtgtgtgtgcccgtgtgtgtgtatgtgtgtgtgtatgtttcttagccccttggattcctgcacgaagtggctgatgggaaccatttcaagacagattgtgaagattgatagaaaatccttcagctaaagtaacagagcatcaaaaacatcactccctctccctccctaacagtgaaaagagagaagggagactctatttaagattcccaaacctaatgatcatctgaatcccgggctaagaatgcagacttttcagactgaccccagaaattctggcccagccaatctagaggcaagcctggccatctgtatttttttttttccaagacagagtcttgctctgttgcccaagctggagtgaagtggtacaatctggctcactgcagcctccgcctcccgggttcaagcgattctcccgcctcagcctcctgagtagctgggattacaggcgcgtatcaccatacccagctaatttttgtatttttagtagagacgggttcaccatgttgcccaggagggtctcgaactcctggcctcaagtgatccaccggcctcggcctcccaaagtgctgggatgacaggcatgaatcactgtgctcagccaccatctggagttttaaaaggctcccatgtgagtccctgtgatggccaggccaggggacccctgccagttctctgtggaagcaaggctggggtcttgggttcctgtatggtggaagctgggtgagccaaggacagggctggctcctctgcccccgctgacgcttcccttgccgttggctttggatgtctttgctgcagtcttctctctggctcaggtgtgggtgggaggggcccacaggaagctcagccttctcctcccaaggtttgagtccctccaaagggcagtgggtggaggaccgggagctttgggtgaccagccactcaaaggaactttctggtcccttcagtatcttcaaggtttggaaactgcaaatgtccccttgatggggaatccgtgtgtgtgtgtgtgtgtgtgtgtgtgtgtgtgtgtgtgtgtgttttctcctagacccgtgacctgagatgtgtgatttttagtcattaaatggaagtgtctgccagctgggcccagcacctaaaaaaaaaaaaaaaaaaa

SEQ ID NO: 13 - DNA, 782 bp, Artificial Sequence
  Description of Artificial Sequence  note  = synthetic construct
ggattcgggctacactttcctcttctccccgaccggagagccgctctttccgcgcggtgcattctggggcccgaggtcgagcccgccgctgccgccgtcgcctgagggaagcgagaagaggccgcgaccgagagaaaaagcggagtcgcaccggagagaagtcgactccctagcagcagccgccgccagagagcccgcccaccagttcgcccgtccccctgccccgttcacaatgcagcctgcttctgcaaagtggtacgatcgaagggactatgtcttcattgaattttgtgttgaagacagtaaggatgttaatgtaaattttgaaaaatccaaacttacattcagttgtctcggaggaagtgataattttaagcatttaaatgaaattgatctttttcactgtattgatccaaatgattccaagcataaaagaacggacagatcaattttatgttgtttacgaaaaggagaatctggccagtcatggccaaggttaacaaaagaaagggcaaagcttaattggcttagtgtcgacttcaataattggaaagactgggaagatgattcagatgaagacatgtctaattttgatcgtttctctgagatgatgaacaacatgggtggtgatgaggatgtagatttaccagaagtagatggagcagatgatgattcacaagacagtgatgatgaaaaaatgccagatctggagtaaggaatattgtcatcacctggattttgagaaagaaaaataacttctctgcaagatttcataattgaga

SEQ ID NO: 14 - DNA, 75 bp, Artificial Sequence
  Description of Artificial Sequence  note  = synthetic construct
atggaaatctgcagaggcctccgcagtcacctaatcactctcctcctcttcctgttccattcagagacgatctgc

SEQ ID NO: 15 - DNA, 655 bp, Artificial Sequence
  Description of Artificial Sequence  note  = synthetic construct
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 16 - DNA, 1278 bp, Artificial Sequence
  Description of Artificial Sequence  note  = synthetic construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgcgcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgttgtaccggcggggtttatatcttcccttctctgttcctccgcagccagccatg

SEQ ID NO: 17 - DNA, 1176 bp, Artificial Sequence
  Description of Artificial Sequence  note  = synthetic construct
cccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcatgcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcgtagcccccgccaggcccggtgggggctggggcgccattgccggtgcgcgctggtcctttgggcgctaactgcgtgcgcgctgggaattggcgctaattgcgcgtgcgcgctgggactcaaggcgctaattgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacttcggctcacagcgcgcccggctattctcgcagctcaccatggatg

SEQ ID NO: 18 - DNA, 1729 bp, Artificial Sequence
  Description of Artificial Sequence  note  = synthetic construct
gaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 19 - DNA, 655 bp, Artificial Sequence; identical to SEQ ID NO: 15
  Description of Artificial Sequence  note  = synthetic construct

SEQ ID NO: 20 - DNA, 366 bp, Artificial Sequence
  Description of Artificial Sequence  note  = synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 21 - DNA, 1295 bp, Artificial Sequence
  Description of Artificial Sequence  note  = synthetic construct
ccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 229 bp, Artificial Sequence
  Description of Artificial Sequence  note  = synthetic construct
gtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 23 - DNA, 281 bp, Artificial Sequence
  Description of Artificial Sequence  note  = synthetic construct
tggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcag

SEQ ID NO: 24 - DNA, 282 bp, Artificial Sequence
  Description of Artificial Sequence  note  = synthetic construct
attatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 25 - DNA, 512 bp, Artificial Sequence
  Description of Artificial Sequence  note  = synthetic construct
ttgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 26 - DNA, 308 bp, Artificial Sequence
  Description of Artificial Sequence  note  = synthetic construct
tcggcgaagcctcgcgcggccggccaggacgaggagcgccactaggttgaacatccgcacgagccgccgggccaggtctcggacgggctctcgagactcgatctcgtgcatgtcggcggtccgcggtgaggttatagaccatctgctaggcgggtccggggagacaggcacattactggcctcggcgcccagcctaggcgtgtctagagctcgaccgcgcgtccggagcgccattcgaccggcgggtagcgagaagaacgccggagaccgcaggttataacaacgtcatgcataaattaagaatgggc

SEQ ID NO: 27 - DNA, 1848 bp, Artificial Sequence
  Description of Artificial Sequence  note  = synthetic construct
ctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctc

SEQ ID NO: 28 - DNA, 49 bp, Artificial Sequence
  Description of Artificial Sequence  note  = synthetic construct
cttctggcgtgtgaccggcggggtttatatcttcccttcccaagcttgg

SEQ ID NO: 29 - DNA, 66 bp, Artificial Sequence
  Description of Artificial Sequence  note  = synthetic construct
cttctggcgtgtgaccggcggggtttatatcttcccttctctgttcctccgcagccccaagcttgg

SEQ ID NO: 30 - DNA, 68 bp, Artificial Sequence
  Description of Artificial Sequence  note  = synthetic construct
cttctggcgtgtgaccggcggggtttatatcttcccttctctgttcctccgcagccagccaagcttgg

SEQ ID NO: 31 - DNA, 69 bp, Artificial Sequence
  Description of Artificial Sequence  note  = synthetic construct
cttctggcgtgtgaccggcggggtttatatcttcccttctctgttcctccgcagccagccatggatgat

SEQ ID NO: 32 - DNA, 1345 bp, Artificial Sequence
  Description of Artificial Sequence  note  = synthetic construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcaagctt

SEQ ID NO: 33 - DNA, 684 bp, Artificial Sequence
  Description of Artificial Sequence  note  = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcgg

SEQ ID NO: 34 - DNA, 2069 bp, Artificial Sequence
  Description of Artificial Sequence  note  = synthetic construct
aattccatacctgcttgatccacatatgaactacagggggacatgatgaggtccagtctaaaggtcactggcaacctctctcaagatctccctcactatgccattattcaggattggggaagatgtggctggagcctaaggggctcttcccttccctatggtgggactcattaggagaacctcagcaagcagtccactgtaagctcaaacaaataccatgtcgctggtatggagtaaggctgttgctatgacaggaactcaggggtcttaactggcttgagcgctgggagggggcagcagccaggccttgtctgtaagctgaagacctggcagtgctgagctggtcaccccccaggacctccttttgtgcccaacgagtgactcaccttgggcatagacataatggtcaggggtgggcacgcagcctgcttcccgctgtgctccaggcctccttcgatgctttccgagaagtctattgagctgggagcttgtactgcacccggggctgacatcctggcatcctgggataaaagcagcccacggggctgcccttgccatatgcctcactggcggcagagaacaaggctctattcagcaagtgccctggagtagacaccagaagcccaagcatgggcagaggaaggcaggggttggggggagcagagctgtctgtgttccagaagcccaaggacacagatggctaaggcgcctgggagggacctgagtggaagagatagatgggcctgaagtctcaagcagcaacagcctcctccccgccattggtgagggtggggtttggtttcccggacctacatatccctcagaggcctggtgtgtaggaatttaaaggaggtaaatctcctgagagaatcaggggtacccaggaagacggggtgttacagaaagactccagcatgcacagccaactcactcaaaactactctgtcaggggctgccgggggccaggctcggggtggggggtgggggggcaaagagaagctggaccagggagaaatggcccactaggctggatatgaggccacagaggggctcaggaaggaagcctgctgtcttaccctattaggatctgcgtgcataccttctgctgtgcactctaaacacacagccagaggctcaagttgaccctggagtcacagagagggctccaaccttagccctccactcctgaactccaggaatgagaagatagagttggagcgattcaggggagaggactctgttgagaatgggggtcacaggaaactgtaatataggttgatcccggaggaagggaataggttcttcaagttcctagcatctcacaggccccagagaaggacagagtggggtggtcctggcttaacaggctctaagaactggaagctgattaccccaccaagctgtcactctctgtctctgtctctgtctctgtgtgtgcgcgctcgtgcacacttatcacacaaatgttcatgtgtgtgcacatagatgagttgacaccagaggtcaacctcaggcactgttgccttggttttctgagagagcatttctctctggacctggaactcgccaattagtgagagccaggaagtctgctgattttcactgcccagcactggagtttacaagtatgcactgtcaacccaggccttttgtattcattctgcagctagaacttgggtgggtcttcatgcttgacaggcaagcaatttatggactaagctgtttcctcggccctctcttgacccatttaccagaaagggggttccttgatcaatggcgaacgcaggctggtgtcccaagaaagccttgactctgggtacagtgacctcagtggggtgagaggagttctccccttagctgggctggggcccagctccaccccctcaggctattcaatgggggtgcttccaggaagtcaggggcagatttagtccaacccgttcctccataaaggccctgacatcccaggagccagcagaggcagggcaggatggagcggagacgcatcacctctgcgcgccgctcctatgcctccgagacggtggtcaggggcctcggtcctagtcgac

SEQ ID NO: 35 - DNA, 3633 bp, Artificial Sequence
  Description of Artificial Sequence  note  = synthetic construct
tctagaatatagaagccaaggatttcaagggtttccttttctctcttcttctttttttttctttttctttttcctgagatggagtttccttttgtagccctgactgtcctggaattcactctatagaccaggctagcctcacacttagtgatctgcctgcctctgcctcttgggtgcctcaggattcaaggcatgaaccaccactacccgaccagggatttcttacacacttctgactggactaaccaggaaagcagagagggagacaggaagaaaatgctcagaaggaaggagtaggattggaggtgagctgggggaacccagactgagccgtgcagaagacaaggaagaagaaagccacccacacacctaggatccacccacagattttgctctgggtacccctgtctggagactgtagggctttgtgatggagggtggggtagtcttcatgccccgtgccctttactccagacctaaatgcccacccccacatacagctgctcgctctctctctcccctgcccttctcccaagagaccagttctccatccctggtctgcagccaaggctgggggcagaagaactttctggaggatttgagtgagaaaagcaagagagcctcaagtagggactggaacctctgggaagggagtgcagaggagacccgggtatgtgccctacctggtacatttatacctgggcagcctctgctcctgttccagacttcagagcccagacgggtcctctccctccctcatgaggggaaacatttggggaaatttggagagagacagaactcagagctcagcactttcctctttctgtttttcttcttgaggaattttttcccccaactgctgatgactttaccattcttgggggtgggggggtggagattctggcttttgctccccctacactccaagtgccggacaaagccctacattccacaagaagccagggcttcagagtttcctaaagatgaggtggcgtggcgagtctcctccctctcccagctccaactccccctcccccagtctccagccctagcctggccagggaggccccgccaggctgggaggagaccccaagcacattcttcctctcgctgtcatgctgcagaaattaaagacacatctctgagctgggtacccgccaatcgtttcaagttgagaagtggcagaggaggtcccgagcttcagctcatgccacgtgtaaaggaagcttggaacccactgcccacaactcctggggcaaaaacctggagtcagacatggggtgaaggctgtcacacggcacagacacgtcaagcaccccccccaattctagtagtctcctagcctccaccagaaccccagacccttgatgtggcagtcaccagtccacacctgttaggctcttgtctcttcttccagatgagcctggggggcgtgggggtgctagatcaggagcagggaaaagtagctttggataagtgcttttcccaatacaaaacccaacaaagagtgggcagatcacactgtgtagtgcttcgtggaaccctaccctagacaactgccttgaacacctattccctctgatgtacaccatccccgtccactgttagggagtgggcatcctttggaactgaccactgtggaaggcaggactttactgagttccggaactaccatctcagcttctcagccccagccttaccctacaggcactggcataggcgggggcagatcctgggccacaagtcactgccacatggttgggataattgatgaagtcctgtccttccattgctgtctccagttctgcttctctggaaactctatatttttccctttaattatagcctctgcagtctccctctgccaccccacccgcaccgcttagcctaactgcccacggccagcgacgtggctccctccccttctgctcccttggtcttttttatttttttttctttgccttcgttgcacaaaactagctcagggagggcgtgaagggggggggagcaatggaatcttggatggtttggaggaggcgggactccttgcttccacgtttacagctctgaagacggctgtgggggaagtgatacaggacgtctatgggccctgagaggagacccctatgcttccctgccacccacacagtttaacaaaatgaagttcctaagtagagtgggggtcaggcagagcacctttgcagggttgatgggagcccagggaaagaaaggacactgtcttttagggacacatttaaatataagccacttttcttgggggacgacaaatgaccctttcctgattgcagaggtggggaacaatggctgagattttcagcaaagaagcgaggacatgaggagtagccttcaaataaagtcactcagctaccaaaaacaagtttctgccacacaccgagttacctaggtgtccccagaccagatccaagtacagtaaggaaagcaggttctctacagagagaacacggctctatggccaatgccttctacctgctctttctggattgatactgctacctaagagggcctctaaccaattcctggctgtagccacagctgacacaagacctttttctaagacatccctggtcacaggcctcctgtagcaaattccagccctgggatggaggtggtcaggaaagagtttatacaagaagacccaggccacagctttaaggactcagaaacccccctgcccacacggctgcccatcataacgcagaaggtttcttctggaaggacaaggatgtcaaacttctccccaagcctaatcctcagagatgtctccctctgttacacctggggctggagaaaggtgggtctttcatggagccacattcatggcagaacagatagccaccccactcctttcaaacaaccacatatctgactcttagtatctgtgaagagatgtctaatttgttcccaaatattcctaccctgcatacctgggcccacaccatgaggtattctcctccctctaacagtcacatctgcttagctgcctggttcttcggatttggagagatgcttgcctaacttattcttccttaggtcttcccaaggatgccagaaagactatgagacatggccaagaggaccttttcccaattgtgcctgacactgaaccctttgtaatgttccccaactcagattcccaattctacatccttctgatttgaggtcccagaaggaaagtgcaaggggcatcccctacccacaatcagtatatcgaggcccagccacactcagtgatagcacctctggcccatgtagatctgggggacaagggtggcagaattgcaaaggggggagggggctgggtggactcctttcccttcctttccctcctcccccctcttcgttccaaattgggggccgggccaggcagttctgattggctgggggccgggctgctggctccccctctccaagaggcagggttcctcccagccctcctccatcaggatggtataaaaggggcccaggccagtcgtcggagcagacgggagtttcacctccggacggagcaggaggcacacggagtgaggccacgcatgagccgaagctaaccccccaccccagccgcaaagagtctacatgtctagggtctag

SEQ ID NO: 36 - DNA, 1404 bp, Artificial Sequence
  Description of Artificial Sequence  note  = synthetic construct
tggtggtggtggacaactaggaaactctggcgctttctcctcccctcacaaaactgagtccagctggagccgcctccagactctctggccagggcctcagagtggtcaacagtccctggccagcgttgctctctccaggctaagggcacccactcccctggagattcctgaacctgggccaggaagagccgaattagacaagtgtctccaatccggctgcgtgcggattttgttgcggtgtccctcggttgtctgcagttcctttagtcccttccctggcctgccccttacacctccacacaggtccccctctgtgtaggaatacaccagaccctctcttagccacacacacctccagtcccccgtctacctagatttttttcatagctagttggatgggggatgggttagggaggctgggtttgcgagcctccaggtgggagttcaccgacaggtactccgcaaaggagctggaaggcaggtctggaaaactgtcccccagatttaggattctgggcagcttccatcagcttatactttggctcccccgcccctaaactccccatccccacctcctttctcccgttacttcgtcctccctcgcctttccagcctcgagtctaaagctccatgcttatgcctctgcaaacaaccccctcccttctaaccccagcagaactccgaggaaaggggccggaggccctcttctcgcctgtggttagagggggcagtgtggcagtcccaagtgggggcgaccggaggccgtctcggtgccccgcccgatcaggccactgggcacatcgggggcgggaagcgggctcaccaaaggggcgactggccttggcaggtgtgggctctggtccgacctgggcaggctccgggggcggggtctcaggttacaacgccacggggggctgggggcggcccgcggtttggttggtttgccagcctttggagcgaccgggagcatataaccggagcctctgctgggagaagcgcagggcgccgctgggctgccgggtctcctgcctcctcctcctgctccgagagcctcctgcatgagggcgaggtagagacccggacccgctccggtctctgccgcctcgccgagcttcgcccgggccaaggctctgcgggcctcgcggtgagccatgattcgcctcggggctccccagtcgctggtgctgctgacgctgctcatcgccacggtcctacaatgtcagggccaggatgcccgtaggtcgcccaccacccctgcctgcttccctgacttgcgacccttctcttcttccctccgtccgagttaggcgccaagtcctaggcgcgtagtgcacaggagaacactgatcctaatcctaattctgctagtgaggagttctgtcgcagcatcctcagtcagagtcg

SEQ ID NO: 37 - DNA, 12745 bp, Artificial Sequence
  Description of Artificial Sequence  note  = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcagtacatttatattggctcatgtccaatatgaccgccatgttgacattgattattgactagttattaatagtaatcaattacggggtcattagttcatagcccatatatggagttccgcgttacataacttacggtaattggcccgcctgctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttggtacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggttctgttaacggtggagggcagtgtagtctgagcagtactcgttgctgccgcgcgcgccaccagacataatagctgacagactaacagactgttcctttccatgggtcttttctgcagtcaccgtcgtcgaagcttatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataactcgatcgaccagagctgagatcctacaggagtccagggctggagagaaaacctctgaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 38 - protein, 177 aa, Artificial Sequence
  Description of Artificial Sequence  note  = synthetic construct
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 39 - protein, 569 aa, Artificial Sequence
  Description of Artificial Sequence  note  = synthetic construct
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 40 - protein, 398 aa, Artificial Sequence
  Description of Artificial Sequence  note  = synthetic construct
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 41 - DNA, 21 bp, Artificial Sequence
  Description of Artificial Sequence  note  = synthetic construct
ggaagcgauaauuuuaagctt

SEQ ID NO: 42 - DNA, 21 bp, Artificial Sequence
  Description of Artificial Sequence  note  = synthetic construct
ggagaauccggccagucautt

SEQ ID NO: 43 - DNA, 21 bp, Artificial Sequence
  Description of Artificial Sequence  note  = synthetic construct
ggguugauuauguaccauutt

SEQ ID NO: 44 - DNA, 21 bp, Artificial Sequence
  Description of Artificial Sequence  note  = synthetic construct
ggcuucacuaaggguugautt

SEQ ID NO: 45 - DNA, 21 bp, Artificial Sequence
  Description of Artificial Sequence  note  = synthetic construct
ggcaguauccuuaugcaugtt

SEQ ID NO: 46 - DNA, 21 bp, Artificial Sequence
  Description of Artificial Sequence  note  = synthetic construct
gcuuuuacaucucuuagcatt

SEQ ID NO: 47 - DNA, 21 bp, Artificial Sequence
  Description of Artificial Sequence  note  = synthetic construct
gggaagaaacaguuaccagtt

SEQ ID NO: 48 - DNA, 21 bp, Artificial Sequence
  Description of Artificial Sequence  note  = synthetic construct
gggcaccaacauccuguuutt

SEQ ID NO: 49 - DNA, 21 bp, Artificial Sequence
  Description of Artificial Sequence  note  = synthetic construct
ggaugggaaacuuaaguactt

SEQ ID NO: 50 - DNA, 21 bp, Artificial Sequence
  Description of Artificial Sequence  note  = synthetic construct
ccuacaacucagcgcaugatt

SEQ ID NO: 51 - DNA, 21 bp, Artificial Sequence
  Description of Artificial Sequence  note  = synthetic construct
gcgcaugacuacaucagcutt

SEQ ID NO: 52 - DNA, 21 bp, Artificial Sequence
  Description of Artificial Sequence  note  = synthetic construct
gcuacgagcaguuuuuauutt

SEQ ID NO: 53 - DNA, 21 bp, Artificial Sequence
  Description of Artificial Sequence  note  = synthetic construct
ggauuugaccaguauaagutt

SEQ ID NO: 54 - DNA, 21 bp, Artificial Sequence
  Description of Artificial Sequence  note  = synthetic construct
gggagucuggaacauugugtt

SEQ ID NO: 55 - DNA, 21 bp, Artificial Sequence
  Description of Artificial Sequence  note  = synthetic construct
gguuuuuaguaucagaacutt

SEQ ID NO: 56 - DNA, 21 bp, Artificial Sequence
  Description of Artificial Sequence  note  = synthetic construct
gcacaggauuugaccaguatt

SEQ ID NO: 57 - DNA, 21 bp, Artificial Sequence
  Description of Artificial Sequence  note  = synthetic construct
gggaaauaaggagcuuccutt

SEQ ID NO: 58 - DNA, 21 bp, Artificial Sequence
  Description of Artificial Sequence  note  = synthetic construct
cccuacaguacuaaucaaatt

SEQ ID NO: 59 - DNA, 21 bp, Artificial Sequence
  Description of Artificial Sequence  note  = synthetic construct
ggcuuuugccaaccccgagtt

SEQ ID NO: 60 - DNA, 59 bp, Artificial Sequence
  Description of Artificial Sequence  note  = synthetic construct
gatccggggttgattatgtaccattttcaagagaaatggtacataatcaaccctttttg

SEQ ID NO: 61 - DNA, 59 bp, Artificial Sequence
  Description of Artificial Sequence  note  = synthetic construct
gccccaactaatacatggtaaaagttctctttaccatgtattagttgggaaaaacttaa

SEQ ID NO: 62 - DNA, 59 bp, Artificial Sequence
  Description of Artificial Sequence  note  = synthetic construct
gatccgggcttttgccaaccccgagttcaagagactcggggttggcaaaagcctttttg

SEQ ID NO: 63 - DNA, 59 bp, Artificial Sequence
  Description of Artificial Sequence  note  = synthetic construct
gcccgaaaacggttggggctcaagttctctgagccccaaccgttttcggaaaaacttaa

SEQ ID NO: 64 - DNA, 59 bp, Artificial Sequence
  Description of Artificial Sequence  note  = synthetic construct
gatccgggatgggaaacttaagtacttcaagagagtacttaagtttcccatcctttttg

SEQ ID NO: 65 - DNA, 59 bp, Artificial Sequence; identical to SEQ ID NO: 64
  Description of Artificial Sequence  note  = synthetic construct

SEQ ID NO: 66 - DNA, 59 bp, Artificial Sequence
  Description of Artificial Sequence  note  = synthetic construct
gatccgggatttgaccagtataagtttcaagagaacttatactggtcaaatcctttttg

SEQ ID NO: 67 - DNA, 59 bp, Artificial Sequence
  Description of Artificial Sequence  note  = synthetic construct
gccctaaactggtcatattcaaagttctcttgaatatgaccagtttaggaaaaacttaa

SEQ ID NO: 68 - protein, 25 aa, Artificial Sequence
  Description of Artificial Sequence  note  = synthetic construct
MEICRGLRSHLITLLLFLFHSETIC

SEQ ID NO: 69 - DNA, 1774 bp, Artificial Sequence
  Description of Artificial Sequence  note  = synthetic construct
ggttcaagtgattctgctgcctcagcctcccaggcgggattacaggtgcctgccaccacgcctggctaatttttttgtctttttagtaaagatgaggtttcaccatgttgggcaggctggtttcaattgctgacctcaagtgagccaccccgcctcagcctccaaaatgctaggattacaggcatgagccaccgcacccagccaagtttgtacatatatttttgactacacttcttaactattcttaggataaattactagaagtgaaaattcttgggtgaagagcttgaggcctttacacacacacacacacacacacacacacacacacaaataggctggatcgagtggctcacacctgtaatctcagcagtttgggaggctgaggaaggaggatcacttgagtccaggaggttgagaatagcctgaacaacatagcaagatcttgtctctacaaaaaagtttaaaaaaaattagctggccatggcagcatgtgcctgtagtaccagctactcggaaggctgaggtaggaggatcgcttgagcccaggaggtgattgaagctgcagtgagctgtgattacaccactgcactccagcctgggcaacagagctagactctgtctctaaaaaaaggcacaaaataatatttaaaaagcaccaggtatgcctgtacttgagttgtctttgttgatggctacaaatgagacagctctggctgaagggcggcttccatttccatgggctggaggaggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagaaattctcacacctcagcctgtccagtagcaggggctacaggcgcgcaccaccatcccagctaattaaaaatatttttttgtagagacagggtctctctatgttgcccaggctggtttcaaactcccaggctcaagcaatcctcctgccttgcctcccaaatgacatcggattacaggcgtgagccactgagcctggcccgtattaatgtttagaacacgaattccaggaggcaggctaagtctattcagcttgttcatatgcttgggccaacccaagaaacaagtgggtgacaaatggcaccttttggatagtggtattgactttgaaagtttgggtcaggagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaaccctggttcactcttccaggttctggctccttccagccatggctctcagagtccttctgttaacaggtgcatgggggtggggtgggggactctgggtggggaggagggtaacttttgggtctgtcataaatagagggccc

SEQ ID NO: 70 - DNA, 20 bp, Artificial Sequence
  Description of Artificial Sequence  note  = synthetic construct
gttccctttctgccagccct

SEQ ID NO: 71 - DNA, 19 bp, Artificial Sequence
  Description of Artificial Sequence  note  = synthetic construct
gtgccgtgagtttcccaga

SEQ ID NO: 72 - DNA, 20 bp, Artificial Sequence
  Description of Artificial Sequence  note  = synthetic construct
cccacttccttgccctctca

SEQ ID NO: 73 - DNA, 19 bp, Artificial Sequence
  Description of Artificial Sequence  note  = synthetic construct
actctgttgtgttcccgca

SEQ ID NO: 74 - DNA, 19 bp, Artificial Sequence
  Description of Artificial Sequence  note  = synthetic construct
cctcttctcgcttccctca

SEQ ID NO: 75 - DNA, 20 bp, Artificial Sequence
  Description of Artificial Sequence  note  = synthetic construct
gttcccatcagccacttcgt

SEQ ID NO: 76 - DNA, 44 bp, Artificial Sequence
  Description of Artificial Sequence  note  = synthetic construct
atcctttgatcugaugaguccgugaggacgaaagttataagcac

SEQ ID NO: 77 - DNA, 43 bp, Artificial Sequence
  Description of Artificial Sequence  note  = synthetic construct
tctttagaacugaugaguccgugaggacgaaagacaaattgca

SEQ ID NO: 78 - DNA, 44 bp, Artificial Sequence
  Description of Artificial Sequence  note  = synthetic construct
ggccgaagcgcugaugaguccgugaggacgaaagacagatgccc

SEQ ID NO: 79 - DNA, 44 bp, Artificial Sequence
  Description of Artificial Sequence  note  = synthetic construct
tggatgtcaacugaugaguccgugaggacgaaagataactcata

SEQ ID NO: 80 - DNA, 44 bp, Artificial Sequence
  Description of Artificial Sequence  note  = synthetic construct
aggagttcgacugaugaguccgugaggacgaaagcctcctgggc

SEQ ID NO: 81 - DNA, 44 bp, Artificial Sequence
  Description of Artificial Sequence  note  = synthetic construct
tctccggtgccugaugaguccgugaggacgaaagtccgcttttt

SEQ ID NO: 82 - DNA, 24 bp, Artificial Sequence
  Description of Artificial Sequence  note  = synthetic construct
cugaugaguccgugaggacgaaag

SEQ ID NO: 83 - protein, 400 aa, Artificial Sequence
  Description of Artificial Sequence  note  = synthetic construct
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 84 - DNA, 1199 bp, Artificial Sequence
  Description of Artificial Sequence  note  = synthetic construct
atggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgcc

SEQ ID NO: 85 - protein, 398 aa, Artificial Sequence
  Description of Artificial Sequence  note  = synthetic construct
MDSSAGPGNISDCSDPLAPASWSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 86 - DNA, 1194 bp, Artificial Sequence
  Description of Artificial Sequence  note  = synthetic construct
atggacagcagcgccggcccagggaacatcagcgactgctctgaccccttagctcctgcaagttggtccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcactttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccc

SEQ ID NO: 87 - DNA, 2423 bp, Artificial Sequence
  Description of Artificial Sequence  note  = synthetic construct
acccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgtctgagtctgcagtcccgagatcccagccatcatgtccatagagaagatctgggcccgggagatcctggactcccgcgggaaccccacagtggaggtggatctctatactgccaaaggtcttttccgggctgcagtgcccagtggagcctctacgggcatctatgaggccctggagctgagggatggagacaaacagcgttacttaggcaaaggtgtcctgaaggcagtggaccacatcaactccaccatcgcgccagccctcatcagctcaggtctctctgtggtggagcaagagaaactggacaacctgatgctggagttggatgggactgagaacaaatccaagtttggggccaatgccatcctgggtgtgtctctggccgtgtgtaaggcaggggcagctgagcgggaactgcccctgtatcgccacattgctcagctggccgggaactcagacctcatcctgcctgtgccggccttcaacgtgatcaatggtggctctcatgctggcaacaagctggccatgcaggagttcatgatcctcccagtgggagctgagagctttcgggatgccatgcgactaggtgcagaggtctaccatacactcaagggagtcatcaaggacaaatacggcaaggatgccaccaatgtgggggatgaaggtggctttgcccccaatatcctggagaacagtgaagccttggagctggtgaaggaagccatcgacaaggctggctacacggaaaagatcgttattggcatggatgttgctgcctcagagttttatcgtgatggcaaatatgacttggacttcaagtctcccactgatccttcccgatacatcactggggaccagctgggggcactctaccaggactttgtcagggactatcctgtggtctccattgaggacccatttgaccaggatgattgggctgcctggtccaagttcacagccaatgtagggatccagattgtgggtgatgacctgacagtgaccaacccaaaacgtattgagcgggcagtggaagaaaaggcctgcaactgtctgctgctcaaggtcaaccagatcggctcggtcactgaagccatccaagcgtgcaagctggcccaggagaatggctggggggtcatggtgagtcatcgctcaggagagactgaggacacattcattgctgacctggtggtggggctgtgcacaggccagatcaagactggtgccccgtgccgttctgaacgtctggctaaatacaaccagctcatgagaattgaggaagagctgggggatgaagctcgctttgccggacataacttccgtaatcccagtgtgctgtgattcctctgcttgcctggagacgtggaacctctgtctcatcctcctggaaccttgctgtcctgatctgtgatagttcaccccctgagatcccctgagccccagggtgcccagaacttccctgattgacctgctccgctgctccttggcttacctgacctcttgctgtctctgctcgccctcctttctgtgccctactcattggggttccgcactttccacttcttcctttctctttctctcttccctcagaaactagaaatgtgaatgaggattattataaaagggggtccgtggaagaatgatcagcatctgtgatgggagcgtcagggttggtgtgctgaggtgttagagagggaccatgtgtcacttgtgctttgctcttgtcccacgtgtcttccactttgcatatgagccgtgaactgtgcatagtgctgggatggaggggagtgttgggcatgtgatcacgcctggctaataaggctttagtgtatttatttatttatttattttatttgtttttcattcatcccattaatcatttccccataactcaatggcctaaaactggcctgacttgggggaacgatgtgtctgtatttcatgtggctgtagatcccaagatgactggggtgggaggtcttgctagaatgggaagggtcatagaaagggccttgacatcagttcctttgtgtgtactcactgaagcctgcgttggtccagagcggaggctgtgtgcctgggggagttttcctctatacatctctccccaaccctaggttccctgttcttcctccagctgcaccagagcaacctctcactccccatgccacgttccacagttgccaccacctctgtggcattgaaatgagcacctccattaaagtctgaatcagtgc

SEQ ID NO: 88 - DNA, 1773 bp, Artificial Sequence
  Description of Artificial Sequence  note  = synthetic construct
ccgaggagcctgcgctgctcctggctcacagcgctccgggcgaggagagcgggcggaccggggggctgggccggtgcgggcggcgaggcaggcggacgaggcgcagagacagcggggcggccggggcgcggcacgcggcgggtcggggccggcctctgccttgccgctcccctcgcgtcggatccccgcgcccaggcagccggtggagagggacgcggcggacgccggcagccatggaaccggccccctccgccggcgccgagctgcagcccccgctcttcgccaacgcctcggacgcctaccctagcgccttccccagcgctggcgccaatgcgtcggggccgccaggcgcgcggagcgcctcgtccctcgccctggcaatcgccatcaccgcgctctactcggccgtgtgcgccgtggggctgctgggcaacgtgcttgtcatgttcggcatcgtccggtacactaagatgaagacggccaccaacatctacatcttcaacctggccttagccgatgcgctggccaccagcacgctgcctttccagagtgccaagtacctgatggagacgtggcccttcggcgagctgctctgcaaggctgtgctctccatcgactactacaatatgttcaccagcatcttcacgctcaccatgatgagtgttgaccgctacatcgctgtctgccaccctgtcaaggccctggacttccgcacgcctgccaaggccaagctgatcaacatctgtatctgggtcctggcctcaggcgttggcgtgcccatcatggtcatggctgtgacccgtccccgggacggggcagtggtgtgcatgctccagttccccagccccagctggtactgggacacggtgaccaagatctgcgtgttcctcttcgccttcgtggtgcccatcctcatcatcaccgtgtgctatggcctcatgctgctgcgcctgcgcagtgtgcgcctgctgtcgggctccaaggagaaggaccgcagcctgcggcgcatcacgcgcatggtgctggtggttgtgggcgccttcgtggtgtgttgggcgcccatccacatcttcgtcatcgtctggacgctggtggacatcgaccggcgcgacccgctggtggtggctgcgctgcacctgtgcatcgcgctgggctacgccaatagcagcctcaaccccgtgctctacgctttcctcgacgagaacttcaagcgctgcttccgccagctctgccgcaagccctgcggccgcccagaccccagcagcttcagccgcgcccgcgaagccacggcccgcgagcgtgtcaccgcctgcaccccgtccgatggtcccggcggtggcgctgccgcctgaccaggccatccggcccccagacgcccctccctagttgtacccggaggccacatgagtcccagtgggaggcgcgagccatgatgtggagtggggccagtagataggtcggagggctttgggaccgccagatggggcctctgtttcggagacgggaccgggccgctagatgggcatggggtgggcctctggtttggggcgaggcagaggacagatcaatggcgcagtgcctctggtctgggtgcccccgtccacggctctaggtggggcgggaaagccagtgactccaggagaggagcgggacctgtggctctacaactgagtccttaaacagggcatctccaggaaggcggggcttcaaccttgagacagcttcggtttctaacttggagccggactttcggagttggggggtccggggccc

SEQ ID NO: 89 - DNA, 9426 bp, Artificial Sequence
  Description of Artificial Sequence  note  = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaaatcaaattcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagctaagcttgatatcgaattccggatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacatctagagaccacccctccacggccaatacagtggatagaactaatcatcagctagaaaatctggaagcagaaactgctccgttgccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggtaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 90 - DNA, 12745 bp, Artificial Sequence; identical to SEQ ID NO: 37
  Description of Artificial Sequence  note  = synthetic construct

SEQ ID NO: 91 - DNA, 1199 bp, Artificial Sequence
  Description of Artificial Sequence  note  = synthetic construct
atggacagcagcgctgcccccacgaacgccagcaattgcactgatgccttggcgtactcaagttgctccccagcacccagccccggttcctggatcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgcc

SEQ ID NO: 92 - protein, 400 aa, Artificial Sequence
  Description of Artificial Sequence  note  = synthetic construct
MDSSAAPTNASNCTDALAYSSCSPAPSPGSWINLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 93 - DNA, 1986 bp, Artificial Sequence
  Description of Artificial Sequence  note  = synthetic construct
tcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagct

SEQ ID NO: 94 - DNA, 5982 bp, Artificial Sequence
  Description of Artificial Sequence  note  = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaaatcaaattagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggtaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 95 - DNA, 13361 bp, Artificial Sequence
  Description of Artificial Sequence  note  = synthetic construct
tggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggcccataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattcgggaaactggaaggcggggcgagctgcagccccagtgaatcaaatgcagcaagcagtaatgccatctgcacctccaatggaggagaaactattggatttataaattataataaagtaggtactactacaacattagaaaagaggccagaaatactcatatttgtaaatggatatcctataaaatttttattagacacaggagcagatataacaattttaaataggagagattttcaagtaaaaaattctatagaaaatggaaggcaaaatatgattggagtaggaggaggaaagagaggaacaaattatattaatgtacatttagagattagagatgaaaattataagacacaatgtatatttggtaatgtttgtgtcttagaagataactcattaatacaaccattattagggagagataatatgattaaattcaatattaggttagtaatggctcaaatttctgataagattccagtagtaaaagtaaaaatgaaggatcctaataaaggacctcaaataaaacaatggccattaacaaatgaaaaaattgaagccttaacagaaatagtagaaagactagaaagagaagggaaagtaaaaagagcagatccaaataatccatggaatacaccagtatttgctataaaaaagaaaagtggaaaatggagaatgctcatagattttagagaattaaacaaactaactgagaaaggagcagaggtccagttgggactacctcatcctgctggtttacaaataaaaaaacaagtaacagtattagatataggggatgcatatttcaccattcctcttgatccagattatgctccttatacagcatttactttacctagaaaaaataatgcgggaccaggaaggagatttgtgtggtgtagtctaccacaaggctggattttaagtccattgatatatcaaagtacattagataatataatacaaccttttattagacaaaatcctcaattagatatttaccaatatatggatgacatttatataggatcaaatttaagtaaaaaggagcataaagaaaaggtagaagaattaagaaaattactattatggtggggatttgaaactccagaagataaattacaggaagaacccccatatacatggatgggttatgaattacatccattaacatggacaatacaacagaaacagttagacattccagaacagcccactctaaatgagttgcaaaaattagcaggaaaaattaattgggctagccaagctattccagacttgagtataaaagcattaactaacatgatgagaggaaatcaaaacctaaattcaacaagacaatggactaaagaagctcgactggaagtacaaaaggcaaaaaaggctatagaagaacaagtacaactaggatactatgaccccagtaaggagttatatgctaaattaagtttggtgggaccacatcaaataagttatcaagtatatcagaaggatccagaaaagatactatggtatggaaaaatgagtagacaaaagaaaaaggcagaaaatacatgtgatatagccttaagagcatgctataagataagagaagagtctattataagaataggaaaagaaccaagatatgaaatacctacttctagagaagcctgggaatcaaatttaattaattcaccatatcttaaggccccacctcctgaggtagaatatatccatgctgctttgaatataaagagagcgttaagtatgataaaagatgctccaataccaggagcagaaacatggtatatagatggaggtagaaagctaggaaaagcagcaaaagcagcctattggacagatacaggaaagtggcaagtgatggaattagaaggcagtaatcagaaggcagaaatacaagcattattattggcattaaaagcaggatcagaggagatgaatattataacagattcacaatatgttataaatattattcttcaacaaccagatatgatggagggaatctggcaagaagttttagaagaattggagaagaaaacagcaatatttatagattgggtcccaggacataaaggtattccaggaaatgaggaagtagataagctttgtcaaacaatgatgataatagaaggggatgggatattagataaaaggtcagaagatgcaggatatgatttattagctgcaaaagaaatacatttattgccaggagaggtaaaagtaataccaacaggggtaaagctaatgttgcctaaaggatattggggattaataataggaaaaagctcgatagggagtaaaggattggatgtattaggaggggtaatagacgaaggatatcgaggtgaaattggagtaataatgattaatgtatcaagaaaatcaatcaccttaatggaacgacaaaagatagcacaattaataatattgccttgtaaacatgaagtattagaacaaggaaaagtagtaatggattcagagagaggagacaatggttatgggtcaacaggagtattctcctcttgggttgacagaattgaggaagcagaaataaatcatgaaaaatttcactcagatccacagtacttaaggactgaatttaatttacctaaaatggtagcagaagagataagacgaaaatgcccagtatgcagaatcagaggagaacaagtgggaggacaattgaaaatagggcctggtatctggcaaatggattgcacacactttgatggcaaaataattcttgtgggtatacatgtggaatcaggatatatatgggcacaaataatttctcaagaaactgctgactgtacagttaaagctgtcttacaattgttgagtgctcataatgttactgaattacaaacagataatggaccaaattttaaaaatcaaaagatggaaggagtactcaattacatgggtgtgaaacataagtttggtatcccagggaacccacagtcacaagcattagttgaaaatgtaaatcatacattaaaagtttggattcggaaatttttgcctgaaacaacctccttggataatgccttatctctcgctgtacatagtctcaattttaaaagaagaggtaggataggagggatggccccttatgaattattagcacaacaagaatccttaagaatacaagattatttttctgcaataccacaaaaattgcaagcacagtggatttattataaagatcaaaaagataagaaatggaaaggaccaatgagagtagaatactggggacagggatcagtattattaaaggatgaagagaagggatattttcttatacctaggagacacataaggagagttccagaaccctgcgctcttcctgaaggggatgagtgaagaagattggcaggtaagtagaagactctttgcagtgctccaaggaggagtaaatagcgctatgctatacatatctaggctacctccggatgaaagagaaaagtataaaaaagacttcaagaaaagactttttgacacagaaacaggatttataaagagactacggaaagctgaaggaataaaatggagctttcatactagagattattacataggatatgtcagagaaatggtggcaggatccactacatcattaagtctaaggatgtatatatatataagtaacccactatggcattctcagtatcgtccaggtttgaaaaatttcaataaggaatggccttttgtaaatatgtggataaaaacaggatttatgtgggatgatattgaaaaacaaaatatttgtataggaggagaagtttcaccaggatggggaccagggatggtaggtatagcaataaaagcttttagttgtggcgaaagaaagattgaggctactcctgtaatgattataagaggagaaatagatccaaaaaaatggtgcggagattgttggaatttaatgtgtcttagaaactcacctccaaagactttacaaagactcgctatgttggcgtgtggcgtgccggctaagaagtggcgaggatgctgtaatcaacgctttgtttctccttacagaacgcctgctgatttagaggtcattcaatccaagcccagctggaacctgttatggtcgggagaattatgaatggaagacataatagtattattcaatagggtcactgagaaactagaaaaagaattagctatcagaatatttgtattagcacatcaattagaaagggacaaagctattagattactacaaggattattttggagatatagatttaagaaaccccgagtagattattgtttatgttggtggtgttgcaaattctattattggcagttgcaatctacattatcaataactactgcttagaaatatttagattaatatttcatttgcaacaataagaatggcagaaggatttgcagccaatagacaatggataggactagaagaagctgaagagttattagattttgatatagcaacacaaatgagtgaagaaggaccactaaatccaggagtaaacccatttagggtacctggaataacagaaaaagaaaagcaaaactactgtaacatattacaacctaagttacaagatctaaggaacgaaattcaagaggtaaaactggaagaaggaaatgcaggtaagtttagaagagcaagatttttaaggtattctgatgaaagtgtattgtccctggttcatgcgttcataggatattgtatatatttaggtaatcgaaataagttaggatctttaagacatgacattgatatagaagcaccccaagaagagtgttataataatagagagaagggtacaactgacaatataaaatatggtagacgatgttgcctaggaacggtgactttgtacctgattttatttataggaataataatatattcacagacaaccaacgctcaggtagtatggagacttccaccattagtagtcccagtagaagaatcagaaataattttttgggattgttgggcaccagaagaacccgcctgtcaggactttcttggggcaatgatacatctaaaagctaagacaaatataagtatacgagagggacctaccttggggaattgggctagagaaatatgggcaacattattcaaaaaggctactagacaatgtagaagaggcagaatatggaaaagatggaatgagactataacaggaccatcaggatgtgctaataacacatgttataatgtttcagtaatagtacctgattatcagtgttatttagatagagtagatacttggttacaagggaaaataaatatatcattatgtctaacaggaggaaaaatgttgtacaataaagttacaaaacaattaagctattgtacagacccattacaaatcccactgatcaattatacatttggacctaatcaaacatgtatgtggaatacttcacaaattcaggaccctgaaataccaaggccgcggccctggcaacccatcaagaagctgtagaaaaggtgactgaagccttaaagataaacaacttaagattagttacattagagcatcaagtactagtaataggattaaaagtagaagctatggaaaaatttttgtatacagctttcgctatgcaagaattaggatgtaatcaaaatcaatttttctgcaaaatccctcctgagttgtggacaaggtataatatgactataaatcaaacaatatggaatcatggaaatataactttgggggaatggtataaccaaacaaaagatttacaacaaaagttttatgaaataataatggacatagaacaaaataatgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgactagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccactc

SEQ ID NO: 96 - DNA, 9569 bp, Artificial Sequence
  Description of Artificial Sequence  note  = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaattccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctgggcaacggagcagtttctgcttccagattttctagctgatgattagttctatccactgtattggccgtggaggggtggtctctagatgttctgacgaattcgagtggagttttgttgctcaatgttggaagaggttgggatacagaactctctgaagcatcgtttgaagttttcatccagaaatgcataaaggactgggttgaggcagctgtttgtgtaacctagagcaatgcagaagtgccaagaaacagtctggaacgtagtttctgggattgtaaccaaggctttaatgatgacgtaaatgtgaatgggagtccagcagacgatgaacacagccaccaccaccagcaccatcctggtgatccttcgaagattcctgtccttttctttggagccagagagcatgcggacactcttgaggcgcaagatcatcagtccatagcacacggtaatgatgagcactggcataatgaaggcgaagatgaaaacacagatcttcagcaggttttcccagtaccaggttggatgagagaatgttagtgtacaatctatggaaccttgcctgtattttgttgtagccatgaacattacaggaagaccaatggctgaagagaggatccagttgcagacattgataattttggcatttcggggagtacggaaatctaaggccttgacagggtggcagactgcaatgtatcgatcaacactcatggtgcagagggtgaatatgctggtgaacatgttatagtaatctatggagatcactatcttgcaaaggatggttccaaatggccatgttcccattaggtaattcacactctggaagggcagggtactggtggctaaggcatctgccagagcaaggttgaaaatgtagatgttggtggcagtcttcatcttggtgtatctgacaatcacatacatgaccaggaagtttccgaagagccccaccacgcacacgatggagtagagggccatgatcgtgatggccgtgatcatggagggactgccggtcggagggcacaggctgtctctcccgcccaggtcggtgcggttcggaccgcatgggtcggacaggttgccatctaagtgggacaagttgacccaggaaccggggctgggtgctggggagcaacttgagtacgccaaggcatcagtgcaattgctggcgttcgtgggggcagcgctgctgtccatggtactgacggccgggcgggcaccgctgtgcgaggtagccaggagcaccgagacttttcgggttccaagcgcctcagccgcttcctttcgccgctgccacagctcctgctgtttcttacagaaaccagtcctggctgagacagagaggagcgtcaggcggaggggaccgagctgagcatctgacattctcctctgcgtatagccccctcccaccttagtagttcacagaggctcatccggaattcgatatcaagcttagcttggccgcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagttcctcgaggactgcagactcagccggtggcggtggcggtggtgacggcggcggcgcagagagagcaggagtggcggcggctgcggctcccagcgcgggcggggggtgggcagggggcggcgcctatagagccgagcgagcgcatgtgacccgggggccccgatgagtcaagagctgcaggcggagacgcacgtacgagcgcgggtggtggtggggaaccgaacgggtgggggaagggcccgggcgatccaggaccaagctggcaaccccataagtacacggatgctcataaccttgtccagccaactcgagggaagcaggcgtccccttattccagtatagtggcaggtactaacccagacagatggagaagcccaccacaggggttcacgaactcgacacagaatataaaaacgcctttttatttgaaagctgaactgatccatcacagcccacctcctcggctcttctctccagacaacccctactagcatcttagtcgcctcggatctgcttgagagctgtgcttaggactttaggtaggcccacagaacaagccacacagagatgaccaccacagaacctgagcctcgggatgtaatggatttccagtctgggacaaaaaggaacatctcccacttcttgtaacagggctatgaatgcggtggtccccactggggtattatgagttcttgaggtcagcatctytggcctcctggaaaaagagaagacaagtgaactgtgtgccctaccggaatgcccacatccccaggcctctcagtctctctgcctcaatctgccctcctttcgcctctccaccccgtgtcctgttatttttagcttcagatttttagtgcaggatcttttccctggcccagctacatcctccatataccaccagccacctccttcccttcatgcgctcagggttcatcccttcctcccagtctctaccagctatgtctgtagagacagcggtcctctgcctccctctctaattcttctcccttctctggtctcttgtgtcttttcttacctcttcctgagcctctctcatctttccttcatatctgagatacccgcagcctctgactctggcctctcctcccgcgtgcccagcctttgacctccgggggctgccttcctgccatcacctttactgtccctgcaggctagaggagccactaagttggcgggaggtacggttccatgtcttggtccgggtcgagttcttgctcctgttcctccttcagcctctgtcggatctcctcagcttctgcccggtcctcgtcctcatagtactctttgtctaggcgctctaggtgtgccatctgcaccagggcctcctggcggtagtcagtctcatcggcacaggggttgtccagcagcaccaaggccctcagcttgggcaggtcccgcagtttggccagctcccccaggtcactgatcatgttactcctgtatatgaggtcgggaggggttaacgcaggaaaatgggctctgagtgttagctacagatcgggcttcctttgtaagaatacctcctggaagccactctgggccctggaagcagtgatggatgaggaccagccttggcatgatgcaattccctcattcctcctccaccctttagagtccctaaatgccgtgcaatgtgttcttttaaaactttggagcagcaaaagctaggcctcatcctgtcttatatttggagaagaggggcaaatgtcccatacaggagcagacattttctgtgtcagccataccaccatagggggttggaaggattgggcgagcagaggaggaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaattcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagtgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 97 - protein, 401 aa, Artificial Sequence
  Description of Artificial Sequence  note  = synthetic construct
MDSGAVPTNASNCTDPFTHPSSCSPAPSPSSWVNFSHLEGNLSDPCGPNRTELGGSDRLCPSAGSPSMITAIIIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDLRTPRNAKIINICNWILSSAIGLPVMFMATTKYRQGSIDCTLTFSHPTWYWENLLKICVFIFAFIMPILIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRIRQNTRDHPSTANTVDRTNHQLENLEAETTPLP

SEQ ID NO: 98 - DNA, 1415 bp, Artificial Sequence
  Description of Artificial Sequence  note  = synthetic construct
gcctgacgctcctctctggctccgccggggttggtcgctgtaagaaataacaggagctgtggcagcggcgaagacgaagcggctcgcgcgtggaacccgaaaagtcagggtgctcgcggttactcccaacgtggtcccagccggcggtcagcaccatggacagcggcgccgtccccacgaacgccagcaactgcactgatcccttcacacacccttcaagttgctccccagcacctagtcccagctcctgggtcaacttctcccacttagaaggcaacctgtccgacccatgcggtccgaaccgcaccgagctgggagggagcgacagactgtgcccttcggccggcagcccttccatgatcacggccatcatcatcatggccctctactccatcgtgtgcgtggtggggctcttcggaaacttcctggtcatgtatgtgattgtcaggtacaccaaaatgaagactgccaccaacatctatattttcaacctcgccctggcagatgccctggcaaccagtaccctgcctttccagagtgtcaattacctgatgggaacatggccgtttggaaccatcctgtgcaagattgtgatctccatagattactacaatatgttcaccagcatattcaccctctgcaccatgagtgtggatcgctacattgcagtctgccatcctgtcaaggccctggatttacgcactccccgtaatgccaagatcatcaacatctgcaactggatcctctcttcagccattggtctgcctgtgatgttcatggcaacgacaaagtaccggcaaggttccatagattgtacactaacattctctcacccaacgtggtactgggaaaacctgctgaaaatctgtgttttcatctttgccttcatcatgcctatcctcatcattacagtgtgttatgggctgatgatcttacgcctcaagagtgtccgcatgctctctggctccaaagaaaaggacaggaacctgcgaagaatcaccaggatggtgctggtggttgtggctgtgttcattgtctgctggacgcccattcacatctacgtcatcattaaagccttgatcacaatcccggaaactactttccagaccgtttcctggcacttctgcattgctctaggttataccaacagttgcctcaaccccgtcctttatgcatttctggatgaaaacttcaaacgatgcttcagagagttctgtatcccaacttcctccaccattgagcagcaaaactccactcgaattcgtcagaacaccagagaccacccctccacagccaatacggtggataggactaaccatcagctagaaaatctggaagcagaaaccactccgttaccctaactgggtctcataccattcagaccctcactgagcttagacgccacatctatatga

SEQ ID NO: 99 - protein, 398 aa, Artificial Sequence
  Description of Artificial Sequence  note  = synthetic construct
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 100 - DNA, 2229 bp, Artificial Sequence
  Description of Artificial Sequence  note  = synthetic construct
cggatccttagcatccccaaagcgcctccgtgtacttctaaggtgggagggggatacaagcagaggagaatatcggacgctcagacgttccattctgcctgccgctcttctctggttccactagggcttgtccttgtaagaaactgacggagcctagggcagctgtgagaggaagaggctggggcgcctggaacccgaacactcttgagtgctctcagttacagcctaccgagtccgcagcaagcattcagaaccatggacagcagcgccggcccagggaacatcagcgactgctctgaccccttagctcctgcaagttggtccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcactttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgccatccagaccctcgctaaacttagaggctgccatctacttggaatcaggttgctgtcagggtttgtgggaggctctggtttcctggaaaagcatctgatcctgcatcattcaaagtcattcctctctggctattcacgctacacgtcagagacactcagactgtgtcaagcactcagaaggaagagactgcaggccactactgaatccagctcatgtacagaaacatccaatggaccacaatactctgtggtatgtgatttgtgatcaacatagaaggtgacccttccctatgtggaatttttaatttcaaggaaatacttatgatctcatcaagggaaaaatagatgtcacttgttaaattcactgtagtgatgcataaaggaaaagctacctctgacctctagcccagtcaccctctatggaaagttccatagggaatatgtgagggaaaatgttgcttccaaattaaattttcacctttatgttatagtctagttaagacatcaggggcatctctgtttcttggttttgtattgtttgaaagaagacatcttcctccctagctgcgtgttgaaaatgaaagggatttaaaacacagtgtcaactgcagaatagttgattctcgcactgaagggggggggctaatcttcccaattctttccatgtcctccaagtgttcacaaggtcaaactcagagtcacccagtaagctcatcatgccaccattctgagcaaaatccttggattcctgctcagaatggtgg

SEQ ID NO: 101 - protein, 398 aa, Artificial Sequence
  Description of Artificial Sequence  note  = synthetic construct
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVGIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRVRQNTREHPSTANTVDRTNHQLENLEAETAPLP

SEQ ID NO: 102 - DNA, 1401 bp, Artificial Sequence
  Description of Artificial Sequence  note  = synthetic construct
cacgagcttctgcctgccgctcttctctggttccactagggctggtccatgtaagaatctgacggagcctagggcagctgtgagaggaagaggctggggcgcgtggaacccgaaaagtctgagtgctctcagttacagcctacctagtccgcagcaggccttcagcaccatggacagcagcaccggcccagggaacaccagcgactgctcagaccccttagctcaggcaagttgctccccagcacctggctcctggctcaacttgtcccacgttgatggcaaccagtccgatccatgcggtctgaaccgcaccgggcttggcgggaacgacagcctgtgccctcagaccggcagcccttccatggtcacagccattaccatcatggccctctactctatcgtgtgtgtagtgggcctcttcggaaacttcctggtcatgtatgtgattgtaagatacaccaaaatgaagactgccaccaacatctacattttcaaccttgctctggcagacgccttagcgaccagtacactgccctttcagagtgtcaactacctgatgggaacatggcccttcggaaccatcctctgcaagatcgtgatctcaatagattactacaacatgttcaccagcatattcaccctctgcaccatgagcgtggaccgctacattgctgtctgccacccagtcaaagccctggatttccgtaccccccgaaatgccaaaatcgtcaacgtctgcaactggatcctctcttctgccatcggtctgcctgtaatgttcatggcaaccacaaaatacaggcaggggtccatagattgcaccctcacgttctcccacccaacctggtactgggagaacctgctcaaaatctgtgtctttatcttcgctttcatcatgccggtcctcatcatcactgtgtgttacggcctgatgatcttacgactcaagagcgttcgcatgctatcgggctccaaagaaaaggacaggaatctgcgcaggatcacccggatggtgctggtggtcgtggctgtatttatcgtctgctggacccccatccacatctacgtcatcatcaaagcgctgatcacgattccagaaaccacatttcagacggtttcctggcacttctgcattgctttgggttacacgaacagctgcctgaatccagttctttacgccttcctggatgaaaacttcaagcgatgcttcagagagttctgcatcccaacctcgtccacgatcgaacagcaaaactccactcgagtccgtcagaacactagggaacatccctccacggctaatacagtggatcgaactaaccaccagctagaaaatctggaggcagaaactgctccattgccctaactgggtctcacaccatccagaccctcgctaagctt

SEQ ID NO: 103 - protein, 401 aa, Artificial Sequence
  Description of Artificial Sequence  note  = synthetic construct
MDSSADPRNASNCTDPFSPSSMCSPVPSPSSWVNFSHLEGNLSDPCIRNRTELGGSDSLCPPTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRNGSIDCALTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTRDHPSTANTVDRTNHQLENLEAETAPLP

SEQ ID NO: 104 - DNA, 1881 bp, Artificial Sequence
  Description of Artificial Sequence  note  = synthetic construct
atgagctgtggtgtacttctaagatgggagggggcaacaagcagaagataatgtcagaagcttagctctccttctgcctgacgctcctctctggctccgcctgggttggcttctgtaagaagtagcaggagccgtggcgggggctggaggaagcggctgaggcgcgtggaacccgaaaagcccgggtgatcgcggttacctcactgcgtggtcccagccgcccagccgtcagcaccatggacagcagcgctgacccccgaaacgccagcaattgcactgatcccttctcgccctcttcaatgtgctccccagtacctagccccagctcctgggtcaacttctcccacttagaaggcaacctgtccgacccatgcattcggaaccgcaccgagctgggcgggagcgacagcctgtgccctccgaccggcagtccttccatggtcacggccatcaccatcatggccctctactccatcgtgtgcgtggtgggtctcttcggaaacttcctggtcatgtatgtgattgtcagatacaccaaaatgaagactgccaccaacatctatattttcaaccttgctctggcggatgccttagccaccagtaccctacccttccagagtgtcaattacctaatgggaacgtggccgtttggaaccatcctctgcaagatcgtgatctccatagattactacaatatgttcaccagcatattcaccctctgtaccatgagcgtggatcgctacatcgccgtctgccatcccgtcaaggccctggacttccgcactccccgcaacgccaaaatcatcaacgtctgcaactggatcctctcttcagccattggtctgcctgtgatgttcatggcaacaacaaagtaccggaatggttccatagattgtgcactaacattctctcacccaacctggtactgggaaaacctgctgaaaatctgtgttttcatctttgccttcatcatgcctgtcctcatcattacggtgtgttatgggctgatgatcttacgcctcaagagtgttcgcatgctctctggctccaaagaaaaggataggaacctgcgaagaatcaccaggatggtgctggtggttgtggctgtgttcattgtctgctggactcccattcacatttacgtcatcattaaagccttgattacaattccagaaactactttccagactgtgtcctggcacttctgcattgctctaggttatacaaacagctgcctgaacccagtcctttatgcatttctggatgaaaacttcaaacgatgcttcagagagttctgtatcccaacctcctccaccattgagcagcaaaactccgctcgaatccgtcaaaacaccagagaccacccctccacggccaacacggtggacaggaccaaccatcagctagaaaatctggaagcagaaactgctccattgccctaaccaggtgtcatgccattcagatcctcaatgagctaagacagccaccatctacgtggaagcaggttgccatgagaatgtgtgggaggcactattttcctaggaaagtgcctgctctgagtcatcaaatctgtttcctctctggccgctctgctctgcacatgagagggacatccaaactaaatcaagcactaggaaggaaagaactaatccacatggagtttgcctgtgcacataatctcaaggaagatgacccatgggaccgaaacatgctgtggtatgtgcgttgaggtcatcctcaaagatggcccttctgtatgtaatgtgctgttttcaagcaaatgtttacgtcctcatcaaagaaaaaatgtcagttgttaaattcaccatagtaacttgtaaaggctacctctgatcgaagcatcttatgtggaaatccaag

SEQ ID NO: 105 - protein, 372 aa, Artificial Sequence
  Description of Artificial Sequence  note  = synthetic construct
MEPAPSAGAELQPPLFANASDAYPSAFPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAA

SEQ ID NO: 106 - DNA, 1773 bp, Artificial Sequence; identical to SEQ ID NO: 88
  Description of Artificial Sequence  note  = synthetic construct

SEQ ID NO: 107 - protein, 228 aa, Artificial Sequence
  Description of Artificial Sequence  note  = synthetic construct
GIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILVITVCYGLMLLRLRSVRLLSGSKEKDRSLRRITRMVLVVVGAFVVCWAPIHIFVIVWTLVDIDRRDPLVVAAL

SEQ ID NO: 108 - protein, 371 aa, Artificial Sequence
  Description of Artificial Sequence  note  = synthetic construct
MEPVPSARAELQFSLLANVSDTFPSAFPSASANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCTLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRAPCGGQEPGSLRRPRQATARERVTACTPSDGPGGGAA

SEQ ID NO: 109 - protein, 372 aa, Artificial Sequence
  Description of Artificial Sequence  note  = synthetic construct
MELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAA

SEQ ID NO: 110 - DNA, 2219 bp, Artificial Sequence
  Description of Artificial Sequence  note  = synthetic construct
ctctaaaggctgggtccctgcgcccagggcgcacggtggagacggacacggcggcgccatggagctggtgccctctgcccgtgcggagctgcagtcctcgcccctcgtcaacctctcggacgcctttcccagcgccttccccagcgcgggcgccaatgcgtcggggtcgccgggagcccgtagtgcctcgtccctcgccctagccatcgccatcaccgcgctctactcggctgtgtgcgcagtggggcttctgggcaacgtgctcgtcatgtttggcatcgtccggtacaccaaattgaagaccgccaccaacatctacatcttcaatctggctttggctgatgcgctggccaccagcacgctgcccttccagagcgccaagtacttgatggaaacgtggccgtttggcgagctgctgtgcaaggctgtgctctccattgactactacaacatgttcactagcatcttcaccctcaccatgatgagcgtggaccgctacattgctgtctgccatcctgtcaaagccctggacttccggacaccagccaaggccaagctgatcaatatatgcatctgggtcttggcttcaggtgtcggggtccccatcatggtcatggcagtgacccaaccccgggatggtgcagtggtatgcatgctccagttccccagtcccagctggtactgggacactgtgaccaagatctgcgtgttcctctttgccttcgtggtgccgatcctcatcatcacggtgtgctatggcctcatgctactgcgcctgcgcagcgtgcgtctgctgtccggttccaaggagaaggaccgcagcctgcggcgcatcacgcgcatggtgctggtggtggtgggcgccttcgtggtgtgctgggcgcccatccacatcttcgtcatcgtctggacgctggtggacatcaatcggcgcgacccacttgtggtggccgcactgcacctgtgcattgcgctgggctacgccaacagcagcctcaacccggttctctacgccttcctggacgagaacttcaagcgctgcttccgccagctctgtcgcacgccctgcggccgccaagaacccggcagtctccgtcgtccccgccaggccaccacgcgtgagcgtgtcactgcctgcaccccctccgacggcccgggcggtggcgctgccgcctgacctacccgaccttccccttaaacgcccctcccaagtgaagtgatccagaggccacaccgagctccctgggaggctgtggccaccaccaggacagctagaattgggcctgcacagaggggaggcctcctgtggggacggggcctgagggatcaaaggctccaggttggaacggtgggggtgaggaagcagagctggtgattcctaaactgtatccattagtaaggcctctccaatgggacagagcctccgccttgagataacatcgggttctggcctttttgaacacccagctccagtccaagacccaaggattccagctccaggaaccaggaggggcagtgatggggtcgatgatttggtttggctgagagtcccagcatttgtgttatggggaggatctctcatcttagagaagataaggggacagggcattcaggcaaggcagcttggggtttggtcaggagataagcgcccccttcccttggggggaggataagtgggggatggtcaacgttggagaagagtcaaagttctcaccacctttctaactactcagctaaactcgttgaggctagggccaacgtgacttctctgtagagaggatacaagccgggcctgatggggcaggcctgtgtaatcccagtcatagtggaggctgaggctggaaaattaaggaccaacagcctgggcaatttagtgtctcaaaataaaatgtaaagagggctgggaatgtagctcagtggtagggtgtttgtgtgaggctctgggatcaataagacaaaacaaccaaccaaccaaaaaccttccaaacaacaaaaccaaccctcaaaccaaaaaactatgtgggtgtctctgagtctggtttgaagagaacccgcagccctgtatccctgtggggctgtggacagtgggcagaagcagaggctccctggatcctgaacaagggccccaaaagcaagttctaaagggacccctgaaaccgagtaagcctttgtgtcaagaagtgggagtacaaccagaaaggtggctgagtgctttagag

SEQ ID NO: 111 - protein, 380 aa, Artificial Sequence
  Description of Artificial Sequence  note  = synthetic construct
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPV

SEQ ID NO: 112 - DNA, 1154 bp, Artificial Sequence
  Description of Artificial Sequence  note  = synthetic construct
atggactccccgatccagatcttccgcggggagccgggccctacctgcgccccgagcgcctgcctgccccccaacagcagcgcctggtttcccggctgggccgagcccgacagcaacggcagcgccggctcggaggacgcgcagctggagcccgcgcacatctccccggccatcccggtcatcatcacggcggtctactccgtagtgttcgtcgtgggcttggtgggcaactcgctggtcatgttcgtgatcatccgatacacaaagatgaagacagcaaccaacatttacatatttaacctggctttggcagatgctttagttactacaaccatgccctttcagagtacggtctacttgatgaattcctggccttttggggatgtgctgtgcaagatagtaatttccattgattactacaacatgttcaccagcatcttcaccttgaccatgatgagcgtggaccgctacattgccgtgtgccaccccgtgaaggctttggacttccgcacacccttgaaggcaaagatcatcaatatctgcatctggctgctgtcgtcatctgttggcatctctgcaatagtccttggaggcaccaaagtcagggaagacgtcgatgtcattgagtgctccttgcagttcccagatgatgactactcctggtgggacctcttcatgaagatctgcgtcttcatctttgccttcgtgatccctgtcctcatcatcatcgtctgctacaccctgatgatcctgcgtctcaagagcgtccggctcctttctggctcccgagagaaagatcgcaacctgcgtaggatcaccagactggtcctggtggtggtggcagtcttcgtcgtctgctggactcccattcacatattcatcctggtggaggctctggggagcacctcccacagcacagctgctctctccagctattacttctgcatcgccttaggctataccaacagtagcctgaatcccattctctacgcctttcttgatgaaaacttcaagcggtgtttccgggacttctgctttccactgaagatgaggatggagcggcagagcactagcagagtccgaaatacagttcaggatcctgcttacctgagggacatcgatgggatgaataaaccagtatgactagtcgtgga

SEQ ID NO: 113 - protein, 380 aa, Artificial Sequence
  Description of Artificial Sequence  note  = synthetic construct
MESPIQIFRGDPGPTCSPSACLLPNSSSWFPNWAESDSNGSVGSEDQQLESAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWLLASSVGISAIVLGGTKVREDVDVIECSLQFPDDEYSWWDLFMKICVFVFAFVIPVLIIIVCYTLMILRLKSVRLLSGSREKDRNLRRITKLVLVVVAVFIICWTPIHIFILVEALGSTSHSTAALSSYYFCIALGYTNSSLNPVLYAFLDENFKRCFRDFCFPIKMRMERQSTNRVRNTVQDPASMRDVGGMNKPV

SEQ ID NO: 114 - DNA, 1410 bp, Artificial Sequence
  Description of Artificial Sequence  note  = synthetic construct
gcgcaccttgctgatcccaaacaggcagagcttcttccagtcttggaaggcacaaattgagcatcaggaacgtggacccatcagggctgaacagctactcaggatctaaagtggtgacttggaaagctgacggtgacttgggaagggaggtcgccaatcagcgatctggagctgcagcgctcaccatggagtcccccattcagatcttccgaggagatccaggccctacctgctctcccagtgcttgccttctccccaacagcagctcttggttccccaactgggcagaatccgacagtaatggcagtgtgggctcagaggatcagcagctggagtccgcgcacatctctccggccatccctgttatcatcaccgctgtctactctgtggtatttgtggtgggcttagtgggcaattctctggtcatgtttgtcatcatccgatacacgaagatgaagaccgcaaccaacatctacatatttaacctggctttggcagatgctttggttactaccactatgccctttcagagtgctgtctacttgatgaattcttggccttttggagatgtgctatgcaagattgtcatttccattgactactacaacatgtttaccagcatattcaccttgaccatgatgagtgtggaccgctacattgctgtgtgccaccctgtgaaagctttggacttccgaacacctttgaaagcaaagatcatcaacatctgcatttggctcctggcatcatctgttggtatatcagcgatagtccttggaggcaccaaagtcagggaagatgtggatgtcattgaatgctccttgcagtttcctgatgatgaatattcctggtgggatctcttcatgaagatctgtgtcttcgtctttgcctttgtgatcccagtcctcatcatcattgtctgctacaccctgatgatcctgcgcctgaagagtgtccggctcctgtctggctcccgagagaaggaccgaaatctccgccgcatcaccaagctggtgctggtagtagttgcagtcttcatcatctgttggacccccattcacatctttatcctggtggaggctctgggaagcacctcccacagcacagctgccctctccagctattatttctgtattgccttgggttataccaacagcagcctgaatcctgttctctatgcctttctggatgaaaacttcaagcggtgttttagggacttctgcttccctattaagatgcgaatggagcgccagagcaccaatagagttagaaacacagttcaggatcctgcttccatgagagatgtgggagggatgaataagccagtatgactagtcgtggaaatgtcttcttattgttctccaggtagagaagagttcaatgatcttggtttaacccagattacaactgcag

SEQ ID NO: 115 - protein, 380 aa, Artificial Sequence
  Description of Artificial Sequence  note  = synthetic construct
MESPIQIFRGEPGPTCAPSACLLPNSSSWFPNWAESDSNGSVGSEDQQLEPAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWLLASSVGISAIVLGGTKVREDVDVIECSLQFPDDEYSWWDLFMKICVFVFAFVIPVLIIIVCYTLMILRLKSVRLLSGSREKDRNLRRITKLVLVVVAVFIICWTPIHIFILVEALGSTSHSTAVLSSYYFCIALGYTNSSLNPVLYAFLDENFKRCFRDFYFPIKMRMERQSTNRVRNTVQDPASMRDVGGMNKPV

SEQ ID NO: 116 - DNA, 2481 bp, Artificial Sequence
  Description of Artificial Sequence  note  = synthetic construct
tccagccctgcctgcacaggcaaagtttgtctctctgggtccaatctgtcctctgctcctgcagcccggcaagtgccatcgctcctcggtttccagctgcagcactcaccatggagtcccccatccagattttccgcggagagccaggccctacctgtgctcccagtgcttgcctactccccaacagcagctcttggttccccaactgggccgaatcggacagcaatggcagtttgggctccgaggaccagcagctggagcccgcgcacatctctccagccatccctgttatcatcaccgctgtctactctgtggtgtttgtggtgggcttagtgggcaattccctggtcatgtttgtcatcatccgatacacaaagatgaagaccgcaaccaacatctacatatttaacctggctttggcagatgctttggttactaccactatgcccttccagagtgctgtctacttgatgaattcttggccttttggagatgttctgtgcaagattgtcatttccattgactactacaacatgtttaccagcatattcaccttgaccatgatgagtgtggaccgctacattgccgtgtgccaccctgtgaaagctttggatttccgaacacctttgaaagcaaagatcatcaacatctgcatttggctactggcatcatctgttggtatatcagcgatagtccttggaggcaccaaagtcagggaagatgtggatgtcattgaatgctccttgcagtttcctgatgatgaatattcctggtgggacctcttcatgaagatctgtgtcttcgtctttgcctttgttatccctgtcttaatcatcattgtctgctacaccctgatgatcctgcgcttgaagagtgtccggctcctctcgggctctcgagagaaggaccgaaatctccgccggatcaccaagctggtgctggtagtggttgcagtcttcatcatctgttggacccccatccacatctttatcctggtcgaggctctaggcagcacctctcacagcacagctgtcctctctagctattacttctgcattgccttgggttataccaacagcagcttgaatcctgttctctatgcctttcttgatgaaaacttcaagcggtgttttagggacttctgcttccccattaagatgcgaatggagcgccagagcacaaacagagttagaaacacagttcaggatcctgcttccatgagggatgtgggtgggatgaataagccagtatgactagtcatggaaatgtcttcctattgttctccgggtagagaagagttcaatgatcttggtttaacccagattaccactgcagtctgaagaggaaagatgaggtattcaataacttagccatgttatgcaatctaaaggtgcagggcacattagtgatctaggctgagtaggggcagcaagtgtgaagaacagagcacatgtcctggcaacaatacacctctttcctaggacagaggagaaggcaatctaacctcaacccttcgataaacagacagcactctttcttctggtccccttgatttactgcacctccatctgcgtggcctttctgtgtaacatagttccaaagctctagagaagaaaatgaaagaaaaagtgcatttgatccaaaacttactgggcacccaaccttcgcgttaacacaggcaaaaccaacttcgtatacaatgaggccataccaagggtcatatccaactcactttctgctggtgatcatgtcttatgtgtgatgagagtgaaacttaggaagaacctggactcagaccctgacactgggggagagcaccatattgacatttgtgaacctatttaaagttgtgggtgttctcgtctcacagtgtctaatgccttgaaaaactacagttgcttcttaaggtctctggtttttagcatgctattcaggaagataatcttctgagaaaacatgaactgatattaaaaggttgaagcttaataccagcaaagtgtgtgtaatttcatctgtaaatagtggtctgtatataaataaggaccaggttttcctgtccagcctgtacatttctcaaggatgccgtagacacacccctggaggcatggaaagttcatgctgggatattttgcttcactataagctactttcttgatttggtcttggtgtgatttctactagattactcaaacattatttactctaacactgatcataacttggtgttaacaattccccaaactttgaattcattctaaagtgttagcattgatcaaatctactttgtggtagcatctgtttgtaaacacacacatattgccagattctctactcaggtagaggaagttgctttgatcatgtacaccttcaaatgttatgctctggctttccacagaaagtggaattgtttcaaaatgcatgctgaaaaaggaaataggatttgagatggcttagcacaatttgcatggtattgagtaagag

SEQ ID NO: 117 - DNA, 2301 bp, Artificial Sequence
  Description of Artificial Sequence  note  = synthetic construct
ccaacagcagctcttggttccccaactgggccgaatcggacagcaatggcagtttgggctccgaggaccagcagctggagcccgcgcacatctctccagccatccctgttatcatcaccgctgtctactctgtggtgtttgtggtgggcttagtgggcaattccctggtcatgtttgtcatcatccgatacacaaagatgaagaccgcaaccaacatctacatatttaacctggctttggcagatgctttggttactaccactatgcccttccagagtgctgtctacttgatgaattcttggccttttggagatgttctgtgcaagattgtcatttccattgactactacaacatgtttaccagcatattcaccttgaccatgatgagtgtggaccgctacattgccgtgtgccaccctgtgaaagctttggatttccgaacacctttgaaagcaaagatcatcaacatctgcatttggctactggcatcatctgttggtatatcagcgatagtccttggaggcaccaaagtcagggaagatgtggatgtcattgaatgctccttgcagtttcctgatgatgaatattcctggtgggacctcttcatgaagatctgtgtcttcgtctttgcctttgttatccctgtcttaatcatcattgtctgctacaccctgatgatcctgcgcttgaagagtgtccggctcctctcgggctctcgagagaaggaccgaaatctccgccggatcaccaagctggtgctggtagtggttgcagtcttcatcatctgttggacccccatccacatctttatcctggtcgaggctctaggcagcacctctcacagcacagctgtcctctctagctattacttctgcattgccttgggttataccaacagcagcttgaatcctgttctctatgcctttcttgatgaaaacttcaagcggtgttttagggacttctgcttccccattaagatgcgaatggagcgccagagcacaaacagagttagaaacacagttcaggatcctgcttccatgagggatgtgggtgggatgaataagccagtatgactagtcatggaaatgtcttcctattgttctccgggtagagaagagttcaatgatcttggtttaacccagattaccactgcagtctgaagaggaaagatgaggtattcaataacttagccatgttatgcaatctaaaggtgcagggcacattagtgatctaggctgagtaggggcagcaagtgtgaagaacagagcacatgtcctggcaacaatacacctctttcctaggacagaggagaaggcaatctaacctcaacccttcgataaacagacagcactctttcttctggtccccttgatttactgcacctccatctgcgtggcctttctgtgtaacatagttccaaagctctagagaagaaaatgaaagaaaaagtgcatttgatccaaaacttactgggcacccaaccttcgcgttaacacaggcaaaaccaacttcgtatacaatgaggccataccaagggtcatatccaactcactttctgctggtgatcatgtcttatgtgtgatgagagtgaaacttaggaagaacctggactcagaccctgacactgggggagagcaccatattgacatttgtgaacctatttaaagttgtgggtgttctcgtctcacagtgtctaatgccttgaaaaactacagttgcttcttaaggtctctggtttttagcatgctattcaggaagataatcttctgagaaaacatgaactgatattaaaaggttgaagcttaataccagcaaagtgtgtgtaatttcatctgtaaatagtggtctgtatataaataaggaccaggttttcctgtccagcctgtacatttctcaaggatgccgtagacacacccctggaggcatggaaagttcatgctgggatattttgcttcactataagctactttcttgatttggtcttggtgtgatttctactagattactcaaacattatttactctaacactgatcataacttggtgttaacaattccccaaactttgaattcattctaaagtgttagcattgatcaaatctactttgtggtagcatctgtttgtaaacacacacatattgccagattctctactcaggtagaggaagttgctttgatcatgtacaccttcaaatgttatgctctggctttccacagaaagtggaattgtttcaaaatgcatgctgaaaaaggaaataggatttgagatggcttagcacaatttgcatggtattgagtaagag

SEQ ID NO: 118 - DNA, 21 bp, Artificial Sequence
  Description of Artificial Sequence  note  = synthetic construct
attttaaaattcaggcctcga

SEQ ID NO: 119 - DNA, 21 bp, Artificial Sequence
  Description of Artificial Sequence  note  = synthetic construct
catagcgttggctacccgtga

SEQ ID NO: 120 - DNA, 21 bp, Artificial Sequence
  Description of Artificial Sequence  note  = synthetic construct
cattctgcagcggtgcacggc

SEQ ID NO: 121 - protein, 31 aa, Artificial Sequence
  Description of Artificial Sequence  note  = synthetic construct
YGGFMTSEKSQTPLVTLFKNAIIKNAYKKGE

SEQ ID NO: 122 - protein, 5 aa, Artificial Sequence
  Description of Artificial Sequence  note  = synthetic construct
YGGFM

SEQ ID NO: 123 - protein, 5 aa, Artificial Sequence
  Description of Artificial Sequence  note  = synthetic construct
YGGFL

SEQ ID NO: 124 - protein, 7 aa, Artificial Sequence
  Description of Artificial Sequence  note  = synthetic construct
YGGFMRF

SEQ ID NO: 125 - protein, 8 aa, Artificial Sequence
  Description of Artificial Sequence  note  = synthetic construct
YGGFMRGL

SEQ ID NO: 126 - protein, 10 aa, Artificial Sequence
  Description of Artificial Sequence  note  = synthetic construct
YGGFMRRVNH

SEQ ID NO: 127 - protein, 17 aa, Artificial Sequence
  Description of Artificial Sequence  note  = synthetic construct
YGGFLRRIRPKLKWDNQ

SEQ ID NO: 128 - protein, 8 aa, Artificial Sequence
  Description of Artificial Sequence  note  = synthetic construct
YGGFLRRI

SEQ ID NO: 129 - protein, 13 aa, Artificial Sequence
  Description of Artificial Sequence  note  = synthetic construct
YGGFLRRQFKVVT

SEQ ID NO: 130 - protein, 10 aa, Artificial Sequence
  Description of Artificial Sequence  note  = synthetic construct
YGGFLRKYPK

SEQ ID NO: 131 - protein, 9 aa, Artificial Sequence
  Description of Artificial Sequence  note  = synthetic construct
YGGFLRKYP

SEQ ID NO: 132 - protein, 17 aa, Artificial Sequence
  Description of Artificial Sequence  note  = synthetic construct
FGGFTGARKSARKLANQ

SEQ ID NO: 133 - protein, 6 aa, Artificial Sequence
  Description of Artificial Sequence  note  = synthetic construct
YPWFNH

SEQ ID NO: 134 - protein, 6 aa, Artificial Sequence
  Description of Artificial Sequence  note  = synthetic construct
YPFFNH

SEQ ID NO: 135 - DNA, 20 bp, Artificial Sequence
  Description of Artificial Sequence  note  = synthetic construct
tgacccccaaggctcaaata

SEQ ID NO: 136 - DNA, 21 bp, Artificial Sequence
  Description of Artificial Sequence  note  = synthetic construct
cccaggtcctcgcttatgatc

SEQ ID NO: 137 - DNA, 27 bp, Artificial Sequence
  Description of Artificial Sequence  note  = synthetic construct
ctttgcccagcacttcacccatcagtt

SEQ ID NO: 138 - DNA, 20 bp, Artificial Sequence
  Description of Artificial Sequence  note  = synthetic construct
tcgctcagggtcacaagaaa

SEQ ID NO: 139 - DNA, 22 bp, Artificial Sequence
  Description of Artificial Sequence  note  = synthetic construct
atcagaggcaaggaggaaacac

SEQ ID NO: 140 - DNA, 29 bp, Artificial Sequence
  Description of Artificial Sequence  note  = synthetic construct
catggcacattctgttcaaagagagcctg

SEQ ID NO: 141 - DNA, 19 bp, Artificial Sequence
  Description of Artificial Sequence  note  = synthetic construct
gacaaggctgccccgacta

SEQ ID NO: 142 - DNA, 26 bp, Artificial Sequence
  Description of Artificial Sequence  note  = synthetic construct
tttctcctggtatgagatagcaaatc

SEQ ID NO: 143 - DNA, 18 bp, Artificial Sequence
  Description of Artificial Sequence  note  = synthetic construct
cccaatgtgtccgtcgtg

SEQ ID NO: 144 - DNA, 20 bp, Artificial Sequence
  Description of Artificial Sequence  note  = synthetic construct
cctgcttcaccaccttcttg

SEQ ID NO: 145 - DNA, 28 bp, Artificial Sequence
  Description of Artificial Sequence  note  = synthetic construct
tgtcatatacttggcaggtttctccagg

SEQ ID NO: 146 - DNA, 23 bp, Artificial Sequence
  Description of Artificial Sequence  note  = synthetic construct
ccagaaaccgctatgaagttcct

SEQ ID NO: 147 - DNA, 20 bp, Artificial Sequence
  Description of Artificial Sequence  note  = synthetic construct
caccagcatcagtcccaaga

SEQ ID NO: 148 - DNA, 27 bp, Artificial Sequence
  Description of Artificial Sequence  note  = synthetic construct
tctgcaagagacttccatccagttgcc